(19) United States
(12) Patent Application Publication
(10) Pub. No.: US 2026/0043051 A1
(43) Pub. Date: Feb. 12, 2026

(54) BIOPRODUCTION OF ISOPRENOIDS

(71) Applicant: INSCRIPTA, INC., Pleasanton, CA (US)

(72) Inventors: Amanda R. Reider Apel, Alameda, CA (US); Douglas Hansen, Sunnyvale, CA (US); Drew Fraser Thacker, Alameda, CA (US)

(21) Appl. No.: 18/995,529

(22) PCT Filed: Jul. 17, 2023
(86) PCT No.: PCT/US23/70315

§ 371 Date: Jan. 16, 2025

Related U.S. Application Data
(60) Provisional application No. 63/390,137, filed on Jul. 18, 2022.

(51) Int. Cl. C12P 5/00; C12N 1/16; C12N 9/04; C12R 1/865

(52) U.S. Cl. CPC C12P 5/007; C12N 1/16; C12N 9/0006; C12R 2001/865; C12Y 101/01034; C12Y 101/01088

(57) ABSTRACT

The present disclosure relates to synthetic biology and, in particular the bioproduction of isoprenoids using heterologous expression of 3-hydroxy-3-methylglutaryl-coenzyme-A reductase (HMGR) enzyme(s).

### CROSS REFERENCE TO RELATED APPLICATIONS

This application claims priority under 35 U.S.C. § 119 to Provisional Application Ser. No. 63/390,137 filed Jul. 18, 2022, the disclosures of which are incorporated herein by reference.

### BACKGROUND

The following discussion is merely provided to aid the reader in understanding the disclosure and is not admitted to describe or constitute prior art thereto.

Isoprenoids are industrially useful compounds used in pharmaceutical products, as biofuels, food additives, and other specialty chemical products. Isopentenyl pyrophosphate (IPP) is an isoprenoid precursor and it serves as an intermediate in isoprenoid biosynthesis pathways, such as the 3-hydroxy-3-methylglutaryl-coenzyme A reductase (HMGR) pathway (commonly called the mevalonate pathway) and the non-mevalonate MEP pathway. Thus, isoprenoid precursors such as IPP, and its isomer dimethylallyl pyrophosphate (DMAPP), are used by a variety of organisms in the biosynthesis of terpenes and isoprenoids.

Yeast uses the mevalonate-dependent (MEV) pathway to convert acetyl coenzyme A (acetyl-CoA) to IPP. During redirection of metabolic flux toward isoprenoid production, the targeted biosynthetic pathway is to be properly balanced and engineered both to route carbon to isoprenoid production and to minimize, or even prevent, buildup of toxic metabolic intermediates over a continuous period of time. Ideally, it would be beneficial for this redirection and balancing to occur with as little metabolic burden as possible (i.e., matching cofactor needs and catalytic rates of enzymes), but in practice such redirection and balancing has been difficult to achieve.

The enzyme HMGR is known as the rate-limiting enzyme in eukaryote sterol and isoprenoid biosynthesis. In yeast, the reason for this is threefold: the native genes contain an N-terminal regulatory domain, the catalytic rate of the enzyme is poor and the enzyme uses NADPH (a limited cofactor). Thus, the native yeast gene is frequently used in industrial biotech, though there are reports of several variant options with improved properties.

### SUMMARY

The present disclosure provides examples generally related to synthetic biology and, in particular, the bioproduction of isoprenoids. Some examples provided herein may be employed to overcome the pre-existing challenges of isoprenoid biosynthesis by addressing the rate limiting step in this biosynthetic pathway by screening and identifying several novel HMGR enzymes that provide increased flux to mevalonate, thereby allowing for improved isoprenoid production.

Thus, the present disclosure provides examples of novel 3-hydroxy-3-methylglutaryl-coenzyme A reductase (HMGR) enzymes. Based on the identification of these enzymes, the present disclosure provides examples of nucleic acids encoding the enzymes disclosed herein, transgenic cells that produce isoprenoids, methods of producing isoprenoids, and bioproduction batches of isoprenoids.

In one aspect, the disclosure provides an isolated enzyme, comprising an amino acid sequence with at least about 90% identity with any one of SEQ ID NOs: 1 (tAgHMGR 543), 2 (tDmHMGR_390), 3 (tEcHMGR_466), 4 (tFfHMGR_610), and 5 (tUnHMGR_487), or a variant thereof with up to 20 amino acids deleted from the N-terminus. Thus, the amino acid sequence has at least about 91%, at least about 92%, at least about 93%, at least about 94%, at least about 95%, at least about 96%, at least about 97%, at least about 98%, at least about 99%, or 100% identity with to any one of SEQ ID NOs: 1, 2, 3, 4, and 5, or a variant thereof with up to 20 amino acids deleted from the N-terminus.

In some implementations, the amino acid sequence comprises SEQ ID NO: 1. In some implementations, the amino acid sequence SEQ ID NO: 2. In some implementations, the amino acid sequence comprises SEQ ID NO: 3. In some implementations, the amino acid sequence comprises SEQ ID NO: 4. In some implementations, the amino acid sequence comprises SEQ ID NO: 5.

In some implementations, the amino acid sequence consists of SEQ ID NO: 1. In some implementations, the amino acid sequence consists of SEQ ID NO: 2. In some implementations, the amino acid sequence consists of SEQ ID NO: 3. In some implementations, the amino acid sequence consists of SEQ ID NO: 4. In some implementations, the amino acid sequence consists of SEQ ID NO: 5.

In another aspect, the present disclosure provides an isolated enzyme, comprising an amino acid sequence with at least about 90% identity but less than 100% identity with SEQ ID NO: 6 (tHMGR 531), or a variant thereof with up to 20 amino acids deleted from the N-terminus. Thus, the amino acid sequence has at least about 91%, at least about 92%, at least about 93%, at least about 94%, at least about 95%, at least about 96%, at least about 97%, at least about 98%, or at least about 99% identity with SEQ ID NO: 6, or a variant thereof with up to 20 amino acids deleted from the N-terminus.

In another aspect, the present disclosure provides a nucleic acid comprising a nucleic acid sequence encoding any of the foregoing isolated enzymes.

In another aspect, the present disclosure provides a transgenic yeast cell, comprising a first nucleic acid encoding a first heterologous 3-hydroxy-3-methylglutaryl-coenzyme A reductase (HMGR) enzyme that (i) lacks an inhibitory domain, (ii) utilizes NAD or NADP as a cofactor, or (iii) a combination thereof. The transgenic yeast may comprises only one copy of the first nucleic acid.

In some implementations, the transgenic yeast may further comprise a second, third, fourth, or fifth nucleic acid encoding a second, third, fourth, or fifth heterologous HMGR enzyme that (i) lacks an inhibitory domain, (ii) utilizes NAD or NADP as a cofactor, or (iii) a combination thereof. In some implementations, the transgenic yeast comprises only one copy of each of the second, third, fourth, or fifth nucleic acid. In some implementations, the first, second, third, fourth, or fifth nucleic acid each independently comprise a different nucleic acid sequence or encode a different heterologous HMGR enzyme.

In some implementations, the heterologous HMGR enzyme(s) (i.e., first, second, third, fourth, or fifth) increase(s) flux to mevalonate compared to a native yeast HMGR enzyme.

In some implementations, the yeast does not comprise multiple copies of nucleic acid sequences encoding a native yeast HMGR enzyme.

In some implementations, the first heterologous HMGR enzyme comprises an amino acid sequence with at least about 90% identity with any one of SEQ ID NOs: 1-5 or 8-16. In other words, the amino acid sequence of the first heterologous HMGR enzyme may have at least about 91%, at least about 92%, at least about 93%, at least about 94%, at least about 95%, at least about 96%, at least about 97%, at least about 98%, at least about 99%, or 100% identity with any one of SEQ ID NOs: 1-5 or 8-16.

In some implementations, the first heterologous HMGR enzyme comprises an amino acid sequence of SEQ ID NO: 1. In some implementations, the first heterologous HMGR enzyme comprises an amino acid sequence of SEQ ID NO: 2. In some implementations, the first heterologous HMGR enzyme comprises an amino acid sequence of SEQ ID NO: 3. In some implementations, the first heterologous HMGR enzyme comprises an amino acid sequence of SEQ ID NO: 4. In some implementations, the first heterologous HMGR enzyme comprises an amino acid sequence of SEQ ID NO: 5. In some implementations, the first heterologous HMGR enzyme comprises an amino acid sequence of SEQ ID NO: 8. In some implementations, the first heterologous HMGR enzyme comprises an amino acid sequence of SEQ ID NO: 9. In some implementations, the first heterologous HMGR enzyme comprises an amino acid sequence of SEQ ID NO: 10. In some implementations, the first heterologous HMGR enzyme comprises an amino acid sequence of SEQ ID NO: 11. In some implementations, the first heterologous HMGR enzyme comprises an amino acid sequence of SEQ ID NO: 12. In some implementations, the first heterologous HMGR enzyme comprises an amino acid sequence of SEQ ID NO: 13. In some implementations, the first heterologous HMGR enzyme comprises an amino acid sequence of SEQ ID NO: 14. In some implementations, the first heterologous HMGR enzyme comprises an amino acid sequence of SEQ ID NO: 15. In some implementations, the first heterologous HMGR enzyme comprises an amino acid sequence of SEQ ID NO: 16.

In some implementations, the first heterologous HMGR enzyme consists of an amino acid sequence of SEQ ID NO: 1. In some implementations, the first heterologous HMGR enzyme consists of an amino acid sequence of SEQ ID NO: 2. In some implementations, the first heterologous HMGR enzyme consists of an amino acid sequence of SEQ ID NO: 3. In some implementations, the first heterologous HMGR enzyme consists of an amino acid sequence of SEQ ID NO: 4. In some implementations, the first heterologous HMGR enzyme consists of an amino acid sequence of SEQ ID NO: 5. In some implementations, the first heterologous HMGR enzyme consists of an amino acid sequence of SEQ ID NO: 8. In some implementations, the first heterologous HMGR enzyme consists of an amino acid sequence of SEQ ID NO: 9. In some implementations, the first heterologous HMGR enzyme consists of an amino acid sequence of SEQ ID NO: 10. In some implementations, the first heterologous HMGR enzyme consists of an amino acid sequence of SEQ ID NO: 11. In some implementations, the first heterologous HMGR enzyme consists of an amino acid sequence of SEQ ID NO: 12. In some implementations, the first heterologous HMGR enzyme consists of an amino acid sequence of SEQ ID NO: 13. In some implementations, the first heterologous HMGR enzyme consists of an amino acid sequence of SEQ ID NO: 14. In some implementations, the first heterologous HMGR enzyme consists of an amino acid sequence of SEQ ID NO: 15. In some implementations, the first heterologous HMGR enzyme consists of an amino acid sequence of SEQ ID NO: 16.

In some implementations, the transgenic yeast may further comprise a single copy of a second nucleic acid encoding a second, different heterologous HMGR enzyme that comprises an amino acid sequence with at least about 90% identity with any one of SEQ ID NOs: 1-16. The amino acid sequence of the second, different heterologous has at least about 91%, at least about 92%, at least about 93%, at least about 94%, at least about 95%, at least about 96%, at least about 97%, at least about 98%, at least about 99%, or 100% identity with any one of SEQ ID NOs: 1-16.

In some implementations, the transgenic yeast may further comprise a single copy of a third nucleic acid encoding a third, different heterologous HMGR enzyme that comprises an amino acid sequence with at least about 90% identity with any one of SEQ ID NOs: 1-16. The amino acid sequence of the third, different heterologous has at least about 91%, at least about 92%, at least about 93%, at least about 94%, at least about 95%, at least about 96%, at least about 97%, at least about 98%, at least about 99%, or 100% identity with any one of SEQ ID NOs: 1-16.

In another aspect, the present disclosure provides a transgenic cell, comprising a transgene encoding a 3-hydroxy-3-methylglutaryl-coenzyme A reductase (HMGR) enzyme comprising an amino acid sequence with at least about 90% identity with any one of SEQ ID NOs: 1-5 or 8-16, or a variant thereof with up to 20 amino acids deleted from the N-terminus. In some implementations, the HMGR enzyme that (i) lacks an inhibitory domain, (ii) utilizes NAD or NADP as a cofactor, or (iii) a combination thereof. In some implementations, the heterologous HMGR enzyme increases flux to mevalonate compared to a native yeast HMGR enzyme.

In some implementations, the yeast does not comprise multiple copies of nucleic acid sequences encoding a native yeast HMGR enzyme.

In some implementations, the transgenic cell is eukaryotic. In some implementations, the transgenic cell can be Saccharomyces cerevisiae (S. cerevisiae) or other yeast species. In some implementations, the transgenic cell is prokaryotic (e.g., E. coli).

In some implementations, the transgenic cell is eukaryotic. the amino acid sequence has at least about 91%, at least about 92%, at least about 93%, at least about 94%, at least about 95%, at least about 96%, at least about 97%, at least about 98%, at least about 99%, or 100% identity with any one of SEQ ID NOs: 1-5 or 8-16, or a variant thereof with up to 20 amino acids deleted from the N-terminus.

In some implementations, the transgene is integrated into the transgenic cell's genome. Alternatively, in some implementations, the transgene is not integrated into the transgenic cell's genome.

In some implementations, the transgenic cell comprises only a single copy of the transgene.

In some implementations, the HMGR enzyme comprises or consists of an amino acid sequence of SEQ ID NO: 1. In some implementations, the HMGR enzyme comprises or consists of an amino acid sequence of SEQ ID NO: 2. In some implementations, the HMGR enzyme comprises or consists of an amino acid sequence of SEQ ID NO: 3. In some implementations, the HMGR enzyme comprises or consists of an amino acid sequence of SEQ ID NO: 4. In some implementations, the HMGR enzyme comprises or consists of an amino acid sequence of SEQ ID NO: 5. In some implementations, the HMGR enzyme comprises or consists of an amino acid sequence of SEQ ID NO: 8. In some implementations, the HMGR enzyme comprises or consists of an amino acid sequence of SEQ ID NO: 9. In some implementations, the HMGR enzyme comprises or consists of an amino acid sequence of SEQ ID NO: 10. In some implementations, the HMGR enzyme comprises or consists of an amino acid sequence of SEQ ID NO: 11. In some implementations, the HMGR enzyme comprises or consists of an amino acid sequence of SEQ ID NO: 12. In some implementations, the HMGR enzyme comprises or consists of an amino acid sequence of SEQ ID NO: 13. In some implementations, the HMGR enzyme comprises or consists of an amino acid sequence of SEQ ID NO: 14. In some implementations, the HMGR enzyme comprises or consists of an amino acid sequence of SEQ ID NO: 15. In some implementations, the HMGR enzyme comprises or consists of an amino acid sequence of SEQ ID NO: 16.

In some implementations, the transgenic cell may further comprise a single copy of a second transgene encoding a second, different HMGR enzyme that (i) lacks an inhibitory domain, (ii) utilizes NAD or NADP as a cofactor, or (iii) a combination thereof. In some implementations, the transgenic cell may further comprise a single copy of a third transgene encoding a third, different HMGR enzyme that (i) lacks an inhibitory domain, (ii) utilizes NAD or NADP as a cofactor, or (iii) a combination thereof.

In another aspect, the present disclosure provides a method of producing an isoprenoid, comprising culturing a transgenic yeast or a transgenic cell as disclosed herein (e.g., a transgenic yeast or transgenic cell of any of the foregoing aspects or implementations). In some implementations, the isoprenoid is a sesquiterpene, a monoterpene, a diterpene, or a meroterpene. In some implementations, the isoprenoid is selected from bakuchiol, farnesene, farnesol, geosmin, geraniol, terpineol, limonene, myrcene, linalool, hinokitiol, pinene, cafestol, kahweol, cembrene, taxadiene, α-bisabolol, α-guaiene, bergamontene, and valencene.

In another aspect, the present disclosure provides a method of producing bakuchiol, comprising culturing a transgenic yeast or a transgenic cell as disclosed herein (e.g., a transgenic yeast or transgenic cell of any of the foregoing aspects or implementations).

In another aspect, the present disclosure provides a method of producing farnesene, comprising culturing a transgenic yeast or a transgenic cell as disclosed herein (e.g., a transgenic yeast or transgenic cell of any of the foregoing aspects or implementations).

In another aspect, the present disclosure provides an isolated enzyme, comprising or consisting of an amino acid sequence with at least about 90% identity with any one of SEQ ID NOs: 8-16. In other words, the amino acid sequence has at least about 91%, at least about 92%, at least about 93%, at least about 94%, at least about 95%, at least about 96%, at least about 97%, at least about 98%, at least about 99%, or 100% identity with any one of SEQ ID NOs: 8-16.

In some implementations, the amino acid sequence comprises or consists of SEQ ID NO: 8. In some implementations, the amino acid sequence comprises or consists of SEQ ID NO: 9. In some implementations, the amino acid sequence comprises or consists of SEQ ID NO: 10. In some implementations, the amino acid sequence comprises or consists of SEQ ID NO: 11. In some implementations, the amino acid sequence comprises or consists of SEQ ID NO: 12. In some implementations, the amino acid sequence comprises or consists of SEQ ID NO: 13. In some implementations, the amino acid sequence comprises or consists of SEQ ID NO: 14. In some implementations, the amino acid sequence comprises or consists of SEQ ID NO: 15. In some implementations, the amino acid sequence comprises or consists of SEQ ID NO: 16.

In another aspect, the present disclosure provides an isolated enzyme, comprising an amino acid sequence with at least about 90% identity but less than 100% identity with SEQ ID NO: 7 (EfMvaE). In other words, the amino acid sequence has at least about 91%, at least about 92%, at least about 93%, at least about 94%, at least about 95%, at least about 96%, at least about 97%, at least about 98%, or at least about 99% identity with SEQ ID NO: 7.

In another aspect, the present disclosure provides a nucleic acid comprising a nucleic acid sequence encoding an isolated enzyme as disclosed herein.

In another aspect, the present disclosure provides an isolated 3-hydroxy-3-methylglutaryl-coenzyme A reductase (HMGR) enzyme as disclosed herein.

In another aspect, the present disclosure provides a transgenic cell capable of producing an isoprenoid as disclosed herein.

In another aspect, the present disclosure provides a method of producing an isoprenoid as disclosed herein.

The foregoing general description and following detailed description are examples and are intended to provide further explanation of the disclosure as claimed. Other objects, advantages, and novel features will be readily apparent to those skilled in the art from the following brief description of the drawings and detailed description of the disclosure.

It should be appreciated that all combinations of the foregoing concepts and additional concepts discussed in greater detail below are contemplated as being part of the inventive subject matter disclosed herein and may be employed in any combination to achieve the benefits described herein.

### BRIEF DESCRIPTION OF DRAWINGS

| FIG. 1 (FIG. 1) shows, in one implementation, targeted LCMS metabolomics of mevalonate as produced by the HMGR enzymes disclosed herein. All 11 heterologs that were tested showed increased production of mevalonate over S. cerevisiae tHMGR control (tHMGR_531).

FIG. 2 (FIG. 2) shows, in one implementation, heterologous expression of HMGRS lead to improved production of terpenes. All 15 tested heterologs showed increased production of farnesene over S. cerevisiae reference that did not express one of the heterologous genes disclosed herein.

FIG. 3 (FIG. 3) shows, in one implementation, heterologous HMGR enzymes are functional in S. cerevisiae across multiple domains of life.

### DETAILED DESCRIPTION

Isoprenoids are a class of chemicals that include many commercially valuable compounds, such as terpenes, that can be produced biosynthetically. The conversion of 3-hydroxy-3-methylglutaryl-coenzyme A (HMG-CoA) to mevalonate is known to be a rate limiting step in most isoprenoid bioproduction pathways. This rate limitation has previously been addressed by transforming yeast or other microbes to express multiple copies of HMGR, the enzyme responsible for the conversion of HMG-CoA to mevalonate, in yeast or other microbes, but the present disclosure provides various alternatives that are more efficient and economical.

In particular, the present disclosure provides novel HMGR enzymes and variants thereof that may be expressed in microbes, such as yeast, to drive production of isoprenoids. The disclosure also disclosure transgenic microbes, such as yeast, that express one or more heterologous HMGR enzyme (or variant thereof) disclosed herein. The disclosure further provides methods of producing isoprenoids in, for example, a bioreactor or fermenter.

### I. Definitions

It is to be understood that the disclosed compositions and methods are not limited to the particular implementations described, and as such may vary. It is also to be understood that the terminology used herein is for the purpose of describing particular implementations only, and is not intended to be limiting. The scope of the present technology will be limited only by the appended claims.

As used herein, certain terms may have the following defined meanings. As used in the specification and claims, the singular form “a,” “an” and “the” include singular and plural references unless the context clearly dictates otherwise. For example, the term “a cell” includes a single cell as well as a plurality of cells, including mixtures thereof.

As used herein, “about” means the recited quantity exactly and small variations within a limited range encompassing plus or minus 10% of the recited quantity. In other words, the limited range encompassed can include ±10%, ±9%, ±L8%, ±L7%, ±L6%, ±5%, ±L4%, ±3%, ±2%, ±1%, ±0.5%, ±0.2%, ±0.1%, +0.05%, or smaller, as well as the recited value itself. Thus, by way of example, “about 10” should be understood to mean “10” and a range no larger than “9-11”.

As used herein, the term “bioproduction” is intended to mean production of a compound (e.g., isoprenoid) by way of biological (e.g., enzymatic) synthesis (as opposed to chemical synthesis). In some implementations, bioproduction may be performed by a transgenic organism or microbe that has been engineered to express enzymes involved in the biological synthesis of a compound of interest (e.g., isoprenoid).

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method processes.

Examples and implementations defined by each of these transition terms are within the scope of this disclosure. Accordingly, it is intended that the methods and compositions can include additional processes and components (comprising) or alternatively including processes and compositions of no significance (consisting essentially of) or alternatively, intending only the stated method processes or compositions (consisting of).

As used herein, the term “protein” is a biological macromolecule comprised of one or more chain of amino acids. An “enzyme” is a type of protein that possesses a biological catalytic activity that accelerates chemical reaction. Thus, for the purposes of this disclosure, enzymes are an example of a protein that can catalyze a reaction, such as the reduction of HMG-CoA to mevalonate.

As used herein, “optional” or “optionally” means that the subsequently described event or circumstance may or may not occur, and that the description includes instances where said event or circumstance occurs and instances where it does not.

For the purpose of the description, a phrase in the form “A/B” or in the form “A and/or B” means (A), (B), or (A and B).

### II. Isoprenoids

The term “isoprenoid” refers to a class of organic compounds composed of two or more units of hydrocarbons, with each unit consisting of five carbon atoms arranged in a specific pattern. The five-carbon unit that constitutes the basic building block of isoprenoids is a hydrocarbon called isoprene. Isoprenoids may contain from two to many hundreds of isoprene units. The carbon backbone of an isoprenoid can have one or more functional chemical groups, such as a hydroxyl and/or a carbonyl group, attached to it.

Isoprenoids play widely varying roles in the physiological processes of plants and animals. In plants, isoprenoids may occur in the essential oils, which may be found in the gummy exudates (oleoresins and latices) of many trees and shrubs. In animals, isoprenoids comprise various oily or waxy substances such as fish liver oils, wool wax, and the yellow pigments in egg yolk, butterfat, feathers, and fish scales. They also have a number of commercial uses. For example, commercially valuable isoprenoids may be used as flavorings, solvents, and raw materials for chemicals. Specific examples include, but are not limited to, menthol, citral, camphor, limonene, and α-pinene.

The term “terpene” refers to compounds that are derivatives of a single isoprene unit, though in some instances the terms terpenes and isoprenoids are used interchangeably. The smallest terpene molecules-those containing 10 carbon atoms—are called monoterpenes. The larger molecules, increased by one isoprene unit at a time, are called sesquiterpenes, diterpenes, triterpenes, and tetraterpenes. The monoterpenes are mostly volatile, which accounts for their fragrances. Terpenes of higher molecular weight are less volatile, although sesquiterpenes contribute to the flavors of some foods.

The presently disclosed enzymes and methods make it possible to bioproduce isoprenoids (e.g., monoterpenes, meroterpenes, sesquiterpenes, diterpenes, triterpenes, and tetraterpenes) more efficiency and effectively than prior methods of bioproduction by improving the kinetics of a rate limiting step in the pathway.

### II. HMGR Proteins and Nucleic Acids

3-hydroxy-3-methylglutaryl-coenzyme A reductase (HMGR) is a type of enzyme that catalyzes the synthesis of mevalonate from 3-hydroxy-3-methylglutaryl-coenzyme A (HMG-CoA; also known as β-hydroxy β-methylglutaryl-CoA). One example of a reduction of HMG-CoA to mevalonate is shown below:

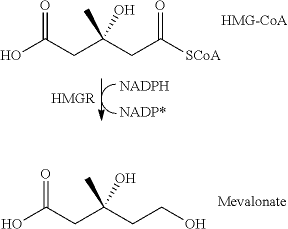

Mevalonate is a precursor to all isoprenoid compounds present in plants, and various HMGR enzymes and homologs have been found throughout the plant and animal kingdoms.

The present disclosure provides isolated HMGR enzymes derived from various plant or animal sources. In particular, this disclosure provides heterologous amino acid sequences of HMGRs and nucleotide sequences encoding HMGRs that increase the flux to mevalonate allowing for robust production of isoprenoids in concert with other MEV pathway enzymes. These HMGRs provide for relative increases in catalytic activity similar to, or beyond, the catalytic activity resulting from an organism expressing an extra copy of an uninhibited, truncated native yeast gene HMGR (e.g., tHMGR_531). Many of these enzymes additionally lack the inhibitory domain, utilize a more abundant cofactor in yeast (i.e., NAD or NADP), or both.

Table 1 provides example amino acid sequences for some of the HMGR enzymes disclosed herein, though it should be understood that the present disclosure is not limited to the specifically disclosed sequences provided in Table 1. For example, the present disclosure also contemplates variants of the sequences disclosed in Table 1 that may be longer, shorter, modified to remove a particular domain (e.g., an inhibitory domain), or otherwise altered to provide a similar protein (e.g., a protein that has at least about 90% a sequence identity with the disclosed sequences) but that retains HMGR activity such that it is able to convert HMVG-CoA to mevalonate.

TABLE 1
Amino Acid Sequences of Certain Disclosed
HMGR Enzymes

| | | SEQ |
|---|---|---|
| | | ID |
| Name | Amino Acid Sequence | NO |
| tAgHMGR_ | MARETIPKSTVSSSETKVVGSVASSIVPSDDETET | 1 |
| 543 | EDEAEPVRPLATLIDVLRKGAVKTLKNKEVVSLVV | |
| | NSELPLYALEKQLGDTTRAVIVRRKALAKLADAPV | |
| | LETERLPYKHYDYDRVFGACCENVIGYMPLPVGVI | |
| | GPLVIDGVAYHIPMATTEGCLVASAMRGCKAINAG | |
| | GGVTTVLTKDGMTRGPCVRFPSLARAGACKLWLDS | |
| | EEGQARVKRAFNSTSRFARLQHVQTALAGDLLFIR | |
| | FRTTTGDAMGMNMISKGVEFALHQMGAEFGWHDME | |
| | IVAVSGNYCTDKKPAAINWIEGRGKSVVAEATVPA | |
| | DVVRKVLKSDVAALVDVNISKNLVGSAMAGAVGGF | |
| | NAHASNLVTALYLALGQDPAQNVESSNCITLMRDV | |
| | GGDLRVSVSMPSIEVGTIGGGTILGPQSAMLDLLG | |
| | VRGPHPSAPGTNARQLAKIVASAVLAGELSLCSAL | |
| | AAGHLVQSHMIHNRAKTPADPEVPCRRPACI | |
| tDmHMGR_ | MDQLRQSGPVAIAAKASQTTPIDEEHVEQEKDTEN | 2 |
| 390 | SAAVRTLLFTIEDQSSANASTQTDLLPLRHRLVGP | |
| | IKPPRPVQECLDILNSTEEGSGPAALSDEEIVSIV | |
| | HAGGTHCPLHKIESVLDDPERGVRIRRQIIGSRAK | |
| | MPVGRLDVLPYEHFDYRKVLNACCENVLGYVPIPV | |
| | GYAGPLLLDGETYYVPMATTEGALVASTNRGCKAL | |
| | SVRGVRSVVEDVGMTRAPCVRFPSVARAAEAKSWI | |
| | ENDENYRVVKTEFDSTSRFGRLKDCHIAMDGPQLY | |
| | IRFVAITGDAMGMNMVSKGAEMALRRIQLQFPDMQ | |
| | IISLSGNFCCDKKPAAINWIKGRGKRVVTECTISA | |
| | ATLRSVLKTDAKTLVECNKLKNMGGSAMAGSIGGN | |
| | NAHAANMVTAVFLATGQDPAQNVTSSNCSTAMECW | |
| | AENSEDLYMTCTMPSLEVGTVGGGTGLPGQSACLE | |
| | MLGVRGAHATRPGDNAKKLAQIVCATVMAGELSLM | |
| | AALVNSDLVKSHMRHNRSSIAVNSANNPLNVTVSS | |
| | CSTIS | |
| tEcHMGR_ | MKVYFQLEDTILSSLRYVSVAIRDRFISKLVLFAL | 3 |
| 466 | AISASINIYLLNIARIHTQFTTNELNSKKKLKKSS | |
| | NFAVGSAPIVAPPSERTSESTVSSSETKIMDSVPS | |
| | SVTVSDDETETEDESEPIRPLETLIEIMKQGGVKT | |
| | LRNRELVSLIVNSELPLYALEKQLCDTTRAVVVRR | |
| | KALAKLADAPALETERLPYKNYDYDRVFGACCENV | |
| | IGYMPLPVGVIGPLVIDGIPYHIPMATTEGCLVAS | |
| | AMRGCKAINAGGGVTTVLTKDGMTRGPCIRFPSLA | |
| | RSGACKIWLDSEDGQNKIKKAFNSTSRFARLQHIQ | |
| | TALAGDLLFIRFRTTTGDAMGMNMISKGVEFSLNQ | |
| | MVEEFGWDDMEIVAVSGNYCTDKKPAAINWIEGRG | |
| | KSVVAEATIPGDVVKKVLKSDVNALVDLNISKNLI | |
| | GSAMAGSVGGFNAHASNLVTAVYLALGQDPAQNVE | |
| | SSNCMTLMKEIDGDLRISVSMPSIEVGTIGGGTIL | |
| | EPQSAMLDLLGVRGPHPTEPGTNARQLAKVVACAV | |
| | MAGELSLCSALAAGHLVQSHMIHNRAKASPTSTEV | |
| | KQDDIPRLQEGSVTCIK | |
| tFfHMGR_ | MVLSKWIVIALALSVALNGYLFNVARWGIKDPNVP | 4 |
| 610 | EHNIDRNELARAQQFNDTGSATLPLGEYVPPTPMR | |
| | TQPSTPAITDDEAEGLHMTKARPANLPNRSNEELE | |
| | KLLSEKRVREMTDEEVISLSMRGKIPGYALEKTLG | |
| | DFTRAVKIRRSIIARNKATTDITHSLDRSKLPYEN | |
| | YNWERVFGACCENVIGYMPLPVGVAGPLVIDGQSY | |
| | FIPMATTEGVLVASASRGCKAINSGGGAITVLTAD | |
| | GMTRGPCVAFETLERAGAAKLWLDSEAGQDMMKKA | |
| | FNSTSRFARLQSMKTALAGTNLYIRFKTTTGDAMG | |
| | MNMISKGVEHALSVMANDGGFDDMQIISVSGNYCT | |
| | DKKAAALNWIDGRGKGVVAEAIIPGEVVRSVLKSD | |
| | VDSLVELNVAKNLIGSAMAGSVGGFNAHAANIVAA | |
| | IFLATGQDPAQVVESANCITIMKNLNGALQISVSM | |
| | PSLEVGTLGGGTILEPQGAMLDILGVRGSHPTNPG | |
| | DNARRLARIIGAAVLAGELSLCSALAAGHLVRAHM | |
| | QHNRSAAPSRSTTPAPPMTPVSLAMTSAQERSAST | |
| | TSMSAAAIQRSK | |
| tUnHMGR_ | MSTIKYHQNEASDVISSSPQKKSQFMETNQPYDNT | 5 |
| 487 | LQTPINIDDEEEGLEICLSKSKSPSKRTQAQLEMM | |
| | LKENQASELDDQELIELSLQGKIPGYALEKKLKDT | |
| | TRAVKIRRAVISRTLTTSQTTGLLEYSKLPYKNYD | |
| | WDRVLGACCENVIGYMPLPLGVAGPIIIDSQSYFI | |
| | PMATTEGVLVASTSRGAKAINAGGGAVTVITGDGM | |
| | TRGPCVSFETLERAGAAKVWLDSEIGQKIITKAFN | |
| | STSRFARLQSIKTALAGTYLYPRFKTTTGDAMGMN | |
| | MISKGVEHALNVMATEAGFEDMQIISVSGNFCTDK | |
| | KPAAINWIDGRGKSVVAEAIIPKDIVKSVLKSTVD | |
| | AMVELNISKNLVGSAMAGSIGGFNAHAANIVTAIF | |
| | LATGQDPAQNVESSNCITLMRNLGGNLQISVSMPS | |
| | IEVGTLGGGTILEPQGAMLDMLGVRGSHPTHPGEN | |
| | ARRLARIIAASVLSGELSLCSALAAGHLVKSHMAH | |
| | NRSAPITRSNTPAQISTHPSMISTNSMREKH | |
| tHMGR_ | MAADQLVKTEVTKKSFTAPVQKASTPVLINKTVIS | 6 |
| 531 | GSKVKSLSSAQSSSSGPSSSSEEDDSRDIESLDKK | |
| | IRPLEELEALLSSGNTKQLKNKEVAALVIHGKLPL | |
| | YALEKKLGDTTRAVAVRRKALSILAEAPVLASDRL | |
| | PYKNYDYDRVFGACCENVIGYMPLPVGVIGPLVID | |
| | GTSYHIPMATTEGCLVASAMRGCKAINAGGGATTV | |
| | LTKDGMTRGPVVRFPTLKRSGACKIWLDSEEGQNA | |
| | IKKAFNSTSRFARLQHIQTCLAGDLLFMRFRTTTG | |
| | DAMGMNMISKGVEYSLKQMVEEYGWEDMEVVSVSG | |
| | NYCTDKKPAAINWIEGRGKSVVAEATIPGDVVRKV | |
| | LKSDVSALVELNIAKNLVGSAMAGSVGGFNAHAAN | |
| | LVTAVFLALGQDPAQNVESSNCITLMKEVDGDLRI | |
| | SVSMPSIEVGTIGGGTVLEPQGAMLDLLGVRGPHA | |
| | TAPGTNARQLARIVACAVLAGELSLCAALAAGHLV | |
| | QSHMTHNRKPAEPTKPNNLDATDINRLKDGSVTCI | |
| | KS | |
| EfMvaE | MKTVVIIDALRTPIGKYKGSLSQVSAVDLGTHVTT | 7 |
| | QLLKRHSTISEEIDQVIFGNVLQAGNGQNPARQIA | |
| | INSGLSHEIPAMTVNEVCGSGMKAVILAKQLIQLG | |
| | EAEVLIAGGIENMSQAPKLQRFNYETESYDAPFSS | |
| | MMYDGLTDAFSGQAMGLTAENVAEKYHVTREEQDQ | |
| | FSVHSQLKAAQAQAEGIFADEIAPLEVSGTLVEKD | |
| | EGIRPNSSVEKLGTLKTVFKEDGTVTAGNASTIND | |
| | GASALIIASQEYAEAHGLPYLAIIRDSVEVGIDPA | |
| | YMGISPIKAIQKLLARNQLTTEEIDLYEINEAFAA | |
| | TSIVVQRELALPEEKVNIYGGGISLGHAIGATGAR | |
| | LLTSLSYQLNQKEKKYGVASLCIGGGLGLAMLLER | |
| | PQQKKNSRFYQMSPEERLASLLNEGQISADTKKEF | |
| | ENTALSSQIANHMIENQISETEVPMGVGLHLTVDE | |
| | TDYLVPMATEEPSVIAALSNGAKIAQGFKTVNQQR | |
| | LMRGQIVFYDVADAESLIDELQVRETEIFQQAELS | |
| | YPSIVKRGGGLRDLQYRAFDESFVSVDFLVDVKDA | |
| | MGANIVNAMLEGVAELFREWFAEQKILFSILSNYA | |
| | TESVVTMKTAIPVSRLSKGSNGREIAEKIVLASRY | |
| | ASLDPYRAVTHNKGIMNGIEAVVLATGNDTRAVSA | |
| | SCHAFAVKEGRYQGLTSWTLDGEQLIGEISVPLAL | |
| | ATVGGATKVLPKSQAAADLLAVTDAKELSRVVAAV | |
| | GLAQNLAALRALVSEGIQKGHMALQARSLAMTVGA | |
| | TGKEVEAVAQQLKROKTMNQDRALAILNDLRKQ | |
| DaHMGR | MVADSRLPNFRALTPAQRRDFLADACGLSDAERAL | 8 |
| | LAAPGALPLALADGMIENVFGSFELPLGVAGNFRV | |
| | NGRDVLVPMAVEEPSVVAAASYMAKLAREDGGFQT | |
| | SSTLPLMRAQVQVLGVTDPHGARLAVLQARAQIIE | |
| | RANSRDKVLIGLGGGCKDIEVHVFPDTPRGPMLVV | |
| | HLIVDVRDAMGANTVNTMAESVAPLVEKITGGSVR | |
| | LRILSNLADLRLARARVRLTPQTLATQDRSGEEII | |
| | EGVLDAYTFAAIDPYRAATHNKGIMNGIDPVIVAT | |
| | GNDWRAVEAGAHAYASRSGSYTSLTRWEKDAGGAL | |
| | VGSIELPMPVGLVGGATKTHPLARLALKIMDLQSA | |
| | QQLGEIAAAVGLAQNLGALRALATEGIQRGHMALH | |
| | ARNIALVAGATGDEVDAVARQLAAEHDVRTDRALE | |
| | VLAALRARA | |
| HvHMGR | MTDAASLADRVREGDLRLHELEAHADADTAAEARR | 9 |
| | LLVESQSGASLDAVGNYGFPAEAAESAIENMVGSI | |
| | QVPMGVAGPVSVDGGSVAGEKYLPLATTEGALLAS | |
| | VNRGCSVINSAGGATARVLKSGMTRAPVFRVADVA | |
| | EAEALVSWTRDNFAALKEAAEETTNHGELLDVTPY | |
| | VVGNSVYLRFRYDTKDAMGMNMATIATEAVCGVVE | |
| | AETAASLVALSGNLCSDKKPAAINAVEGRGRSVTA | |
| | DVRIPREVVEERLHTTPEAVAELNTRKNLVGSAKA | |
| | ASLGFNAHVANVVAAMFLATGQDEAQVVEGANAIT | |
| | TAEVQDGDLYVSVSIASLEVGTVGGGTKLPTQSEG | |
| | LDILGVSGGGDPAGSNADALAECIAVGSLAGELSL | |
| | LSALASRHLSSAHAELGR | |
| LkMvaE | MKEVVIIDAARTPIGKYKGSLSSFSAVELGTMVTK | 10 |
| | KLLEKASIKKDEINQVIFGNVLQAGNGQNVARQIS | |
| | IISDIPVDVPAMTINEVCGSGMKAVILARQLIQLG | |
| | EADLVIAGGTESMTRAPLLQQFDSETTSYNGPISS | |
| | MVNDGLTDTFSNTHMGLTAENVAEQFGVTRKEQDQ | |
| | YALDSQLKAAKATENNVFKEEIIPVTLPDGTLLEN | |
| | DEAIRGNSSLEKLGTLKTVFSENGTVTAGNASPLN | |
| | DGASVMILASKEYALKNDLPYLATIKGVAEIGIDP | |
| | SIMGIAPINAINSLLEKTDVSLDAIDRFEINEAFA | |
| | ASSIVVNRELQLDPEKVNSDGGAIALGHPIGASGA | |
| | RILTTLSYGLQRNEQKYGIASLCIGGGLGLAVLLE | |
| | ANQEKAGSFNEKKKFYQLTPEERRSQLVRGGVISK | |
| | ESADQLKNERLSEDIANHLIENQISQVEIPMGVAQ | |
| | NFQINGEKKWVPMATEEPSVIAAASNGAKICGNIT | |
| | AKTPQRLMRGQIVLTGKSEYQAIIEAIDTRKDELF | |
| | LCANNSYPSIVKRGGGVRDISTREFMGSDHAYVSI | |
| | DFLIDVKDAMGANIVNAILEGVASQLRSWFPDEEI | |
| | LFSILSNLATESLATACCTIPFEYLGKSKEAGRQV | |
| | AEKIQQAAEYAKLDVYRAATHNKGIMNGIEAVILA | |
| | TGNDTRAASAAIHAYASRNGFYQGLTDWKIVDGQL | |
| | VGKLTVPLAVATVGGASKILPKAKLALEILDVSSA | |
| | KELAQVIAAVGLAQNLAALKALVTEGIQKGHMSLQ | |
| | ARALAITVGATGDEIEQVASYLRKADTMNQQLASD | |
| | YLLETRS | |
| MbHMGR | MASKTETTMKEDELLEKVVSGEMPLRKIDAYTDTD | 11 |
| | TAVRVRKCAIEKMNGVKFEHIQNYTIDAEAATKRN | |
| | IENMIGTIQIPLGVAGAIMVNGEYASGEFMLPLAT | |
| | TEGALVASVNRGCTVITASGGSNVRIFQDLMTRAP | |
| | VFKLENVNKVKEFVDWVKREETFTNMKEKAGETTR | |
| | FGELLSVDPFITGNTVFLRFAYDTKDAMGMNMVTI | |
| | ATDAVLNFISEDFGVYPISLSGNMCTDKKPAAINN | |
| | ILGRGKTVAADVTIPKEIVEKKLKTTPKMMEEVNY | |
| | RKNLLGSARAGALGFNAHAANIIAALYLACGQDAA | |
| | HVVEGSSAITTMEVNENGDLYCSVTLPSIQVGTVG | |
| | GGTGIATQRDCLNLLGVAGAGEVPGHNSKKLAEII | |
| | AAAVLAGEISLIGAQAAGHLAKAHAELGR | |
| MvHMGR | MFLKDNDLTEDEKLLLQKVLDGDIAFRKIEEFADP | 12 |
| | LTAVKIRRLAIQEYAKLEFEHIQNFSLDVETVTKK | |
| | NIENMIGAVQIPLGVAGLLKVNGEYADAEYYIPLA | |
| | TTEGALVASVNRGCSVITKSGGANVRVFEDEMTRA | |
| | PVFKLESLDRTKKFYEWVKSPEIFEQMKTVAEKTT | |
| | RFGKLLSVKPFVTGTYVYLRFSYDTKDAMGMNMVT | |
| | IATDAVMHLIEDEFGAHPITLSGNMCTDKKPASIS | |
| | TILGRGKTVVAEVTIPEEIVKETLKCTPDAMFEVN | |
| | YSKNLLGSARAGALGFNAHAANVIAAVYLACGQDA | |
| | AHVVEGSTAITSMELTKYGEIHCSVTLPALPVGTV | |
| | GGGTGLGTQRDCLNILGVAGTGDIPGINSRKFAEI | |
| | VASAVLAGEISLIGAQAAGHLARAHAQLGRGKF | |
| Pmev_ | MSLDSRLPAFRNLSPAARLDHIGQLLGLSHDDVSL | 13 |
| MvaA | LANAGALPMDIANGMIENVIGTFELPYAVASNFQI | |
| | NGRDVLVPLVVEEPSIVAAASYMAKLARANGGFTT | |
| | SSSAPLMHAQVQIVGIQDPLNARLSLLRRKDEIIE | |
| | LANRKDQLLNSLGGGCRDIEVHTFADTPRGPMLVA | |
| | HLIVDVRDAMGANTVNTMAEAVAPLMEAITGGQVR | |
| | LRILSNLADLRLARAQVRITPQQLETAEFSGEAVI | |
| | EGILDAYAFAAVDPYRAATHINKGIMNGIDPLIVA | |
| | TGNDWRAVEAGAHAYACRSGHYGSLTTWEKDNNGH | |
| | LVGTLEMPMPVGLVGGATKTHPLAQLSLRILGVKT | |
| | AQALAEIAVAVGLAQNLGAMRALATEGIQRGHMAL | |
| | HARNIAVVAGARGDEVDWVARQLVEYHDVRADRAV | |
| | ALLKQKRGQ | |
| StMvaA | MTKLSWTGFSKKTLQERKEHLKNNALLSQENQDLL | 14 |
| | DNDQQLTLETANQMAENVIGRFTLPFAICPDVLVD | |
| | GVTYQVPMVTEEPSVVAAASYASKLIKRSGGFTTK | |
| | IHDRQMIGQVALFDVPDKATAASKIQAASQKLIDI | |
| | AKEAYPSIVKRGGGPRKLWTETKGDFLIVYLAVDT | |
| | QEAMGANMVNTMMEALVPELENLSEGQSLMAILSN | |
| | LATESLVTATCRLNTRFLSRNKAEAHNFAKKMELA | |
| | SQLAQVDPYRAATHNKGIFNGIDALVIATGNDWRA | |
| | VEAGCHAYASKDGSYRGLSTWTYNQETKELVGELT | |
| | LPMPIATRGGSIGLNPSVSIAHDLLNHPDARTLAG | |
| | IIVSLGLVQNLAALKALTSTGIQAGHMKLQAKSLA | |
| | LLAGANPEEMPHVLSELLKAKHMNQETAQAILEKL | |
| | RNP | |
| ThMvaE | MKDVVIIDALRTPVGKYQGSLSQLSAVELGSAVSK | 15 |
| | KLINNNKKAAAAINQVIFGNVLQAGSGQNPARQIT | |
| | LNSGLSESVYASTINEVCGSGMKAISLASQAIFLD | |
| | EAEVVLAGGTESMSQAPYLSYYNQQEDTYSQPKPA | |
| | MLSDGLTDVFSGQHMGLTAENVAEKFNITRKMQDA | |
| | FALRSQERAANAQEKGYFSNEILPLDIAGKKVDKD | |
| | EGVRKDTSLEKLAKLKTVFKKEGTVTAGNASTIND | |
| | GASAVLLASKNFALANDLSYLAVLKDVVEVGVDPK | |
| | VMGISPIKAIRQLLERNALAIENIDLFEINEAFAS | |
| | SSIA VEQELEIPEDKVNVCGSGISIGHAIGASGA | |
| | RIITTACHQLERVDGRYAVVSLCVGGGLGLAALIE | |
| | RPKANKSHKFYQLTRKERLDFLVSHNKITSKTVDE | |
| | LERTVLPESIAGNLTENQMSEISLPMGLVSNMSVN | |
| | QKDYFVPMATEEPSVVAACNNGVQMAKSSGGFTAV | |
| | MKKKEIRGQIVLMNVTDKSTVIEQIEKNEAEIIST | |
| | AEQSYPSIVKRGGGVKRVVVREFAEDPNFLSVDLI | |
| | VDTQDAMGANMLNTMLEAVATLFRQWFSEEILFSI | |
| | LSNYATDALVSAECYISFASLGKGDAEKGEKIAEK | |
| | IAAASNFAQIDPFRAATHNKGIMNGIDAVVLATGN | |
| | DTRSVNSAVHAYAAKNGKYQGLSQWEIVDNQLKGS | |
| | IELPLAVATAGGATKVLPKAQAALQILDVNDAKEL | |
| | AEVIASVGLAQNLAALKALVTEGIQKGHMALQART | |
| | LALSVGAKDSEVQKVANRLKRQQMNEENARKILQE | |
| | LRNR | |
| ZmHMGR | MEVRGGVGQGSAARHPPAPEPSRAAARVQAGDALP | 16 |
| | LPIRHTNLIFSALFAASLAYLMRRWREKIRSSTPL | |
| | HAVGLAEMLAIFGLVASLIYLLSFFGIAFVQSIVS | |
| | SGDDDEDFLVGSGSSGSAAAPSRQHAQAPAPCELL | |
| | GSPAAAPEKMPEDDEEIVASVVAGKVPSYALEARL | |
| | GDCRRAAGIRREALRRITGRDIEGLPLDGFDYASI | |
| | LGQCCELPVGYVQLPVGVAGPLLLDGRRFYLPMAT | |
| | TEGCLVASTNRGCKAIAESGGATSVVLRDAMTRAP | |
| | VARFPTARRAAELKAFLEDPANFDTLSVVFNRSSR | |
| | FARLQGVQCAMAGRNLYMRFSCSTGDAMGMNMVSK | |
| | GVQNVLDFLQDDFHDMDVISISGNFCSDKKPSAVN | |
| | WIEGRGKSVVCEAVIGEEVVKKVLKTDVQSLVELN | |
| | TIKNLAGSAVAGALGGFNAHASNIVTAIFIATGQD | |
| | PAQNVESSHCITMLEPVNAGRDLHISVTMPSIEVG | |
| | TVGGGTQLASQSACLDLLGVRGASRDRPGSNARLL | |
| | ATVVAGGVLAGELSLLSALAAGQLVKSHMKYNRSS | |
| | KDVSSTTATEKTRQREVDV | |

The present disclosure provides additional example putative HMGR enzymes capable of converting HMG-CoA to mevalonate. Thus, the present disclosure provides proteins that have at least about 65%—e.g., at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 1. In some implementations, a protein disclosed herein may have an amino acid sequence that comprises SEQ ID NO: 1. In some implementations, a protein disclosed herein may have an amino acid sequence that consists of SEQ ID NO: 1. In some implementations, a protein of the present disclosure may consist of 486 amino acids that have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 1. In some implementations, a protein of the present disclosure may comprise more than 486 amino acids, but wherein about 486 of the total amino acids have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 1.

The present disclosure provides proteins that have at least about 65%—e.g., at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 2. In some implementations, a protein disclosed herein may have an amino acid sequence that comprises SEQ ID NO: 2. In some implementations, a protein disclosed herein may have an amino acid sequence that consists of SEQ ID NO: 2. In some implementations, a protein of the present disclosure may consist of 530 amino acids that have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 2. In some implementations, a protein of the present disclosure may comprise more than 530 amino acids, but wherein about 530 of the total amino acids have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 2.

The present disclosure provides proteins that have at least about 65%—e.g., at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 3. In some implementations, a protein disclosed herein may have an amino acid sequence that comprises SEQ ID NO: 3. In some implementations, a protein disclosed herein may have an amino acid sequence that consists of SEQ ID NO: 3. In some implementations, a protein of the present disclosure may consist of 577 amino acids that have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 3. In some implementations, a protein of the present disclosure may comprise more than 577 amino acids, but wherein about 577 of the total amino acids have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 3.

The present disclosure provides proteins that have at least about 65%—e.g., at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 4. In some implementations, a protein disclosed herein may have an amino acid sequence that comprises SEQ ID NO: 4. In some implementations, a protein disclosed herein may have an amino acid sequence that consists of SEQ ID NO: 4. In some implementations, a protein of the present disclosure may consist of 572 amino acids that have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 4. In some implementations, a protein of the present disclosure may comprise more than 572 amino acids, but wherein about 572 of the total amino acids have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 4.

The present disclosure provides proteins that have at least about 65%—e.g., at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 5. In some implementations, a protein disclosed herein may have an amino acid sequence that comprises SEQ ID NO: 5. In some implementations, a protein disclosed herein may have an amino acid sequence that consists of SEQ ID NO: 5. In some implementations, a protein of the present disclosure may consist of 527 amino acids that have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 5. In some implementations, a protein of the present disclosure may comprise more than 527 amino acids, but wherein about 527 of the total amino acids have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 5.

The present disclosure provides proteins that have at least about 65%—e.g., at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 6. In some implementations, a protein disclosed herein may have an amino acid sequence that comprises SEQ ID NO: 6. In some implementations, a protein disclosed herein may have an amino acid sequence that consists of SEQ ID NO: 6. In some implementations, a protein of the present disclosure may consist of 521 amino acids that have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 6. In some implementations, a protein of the present disclosure may comprise more than 521 amino acids, but wherein about 521 of the total amino acids have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 6.

The present disclosure provides proteins that have at least about 65%—e.g., at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 7. In some implementations, a protein disclosed herein may have an amino acid sequence that comprises SEQ ID NO: 7. In some implementations, a protein disclosed herein may have an amino acid sequence that consists of SEQ ID NO: 7. In some implementations, a protein of the present disclosure may consist of 429 amino acids that have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 7. In some implementations, a protein of the present disclosure may comprise more than 429 amino acids, but wherein about 429 of the total amino acids have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 7.

The present disclosure provides proteins that have at least about 65%—e.g., at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 8. In some implementations, a protein disclosed herein may have an amino acid sequence that comprises SEQ ID NO: 8. In some implementations, a protein disclosed herein may have an amino acid sequence that consists of SEQ ID NO: 8. In some implementations, a protein of the present disclosure may consist of 803 amino acids that have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 8. In some implementations, a protein of the present disclosure may comprise more than 803 amino acids, but wherein about 803 of the total amino acids have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 8.

The present disclosure provides proteins that have at least about 65%—e.g., at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 9. In some implementations, a protein disclosed herein may have an amino acid sequence that comprises SEQ ID NO: 9. In some implementations, a protein disclosed herein may have an amino acid sequence that consists of SEQ ID NO: 9. In some implementations, a protein of the present disclosure may consist of 403 amino acids that have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 9. In some implementations, a protein of the present disclosure may comprise more than 403 amino acids, but wherein about 403 of the total amino acids have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 9.

The present disclosure provides proteins that have at least about 65%—e.g., at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 10. In some implementations, a protein disclosed herein may have an amino acid sequence that comprises SEQ ID NO: 10. In some implementations, a protein disclosed herein may have an amino acid sequence that consists of SEQ ID NO: 10. In some implementations, a protein of the present disclosure may consist of 812 amino acids that have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 10. In some implementations, a protein of the present disclosure may comprise more than 812 amino acids, but wherein about 812 of the total amino acids have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 10.

The present disclosure provides proteins that have at least about 65%—e.g., at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 11. In some implementations, a protein disclosed herein may have an amino acid sequence that comprises SEQ ID NO: 11. In some implementations, a protein disclosed herein may have an amino acid sequence that consists of SEQ ID NO: 11. In some implementations, a protein of the present disclosure may consist of 414 amino acids that have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 11. In some implementations, a protein of the present disclosure may comprise more than 414 amino acids, but wherein about 414 of the total amino acids have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 11.

The present disclosure provides proteins that have at least about 65%—e.g., at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 12. In some implementations, a protein disclosed herein may have an amino acid sequence that comprises SEQ ID NO: 12. In some implementations, a protein disclosed herein may have an amino acid sequence that consists of SEQ ID NO: 12. In some implementations, a protein of the present disclosure may consist of 418 amino acids that have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 12. In some implementations, a protein of the present disclosure may comprise more than 418 amino acids, but wherein about 418 of the total amino acids have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 12.

The present disclosure provides proteins that have at least about 65%—e.g., at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 13. In some implementations, a protein disclosed herein may have an amino acid sequence that comprises SEQ ID NO: 13. In some implementations, a protein disclosed herein may have an amino acid sequence that consists of SEQ ID NO: 13. In some implementations, a protein of the present disclosure may consist of 428 amino acids that have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 13. In some implementations, a protein of the present disclosure may comprise more than 428 amino acids, but wherein about 428 of the total amino acids have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 13.

The present disclosure provides proteins that have at least about 65%—e.g., at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 14. In some implementations, a protein disclosed herein may have an amino acid sequence that comprises SEQ ID NO: 14. In some implementations, a protein disclosed herein may have an amino acid sequence that consists of SEQ ID NO: 14. In some implementations, a protein of the present disclosure may consist of 423 amino acids that have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 14. In some implementations, a protein of the present disclosure may comprise more than 423 amino acids, but wherein about 423 of the total amino acids have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 14.

The present disclosure provides proteins that have at least about 65%—e.g., at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 15. In some implementations, a protein disclosed herein may have an amino acid sequence that comprises SEQ ID NO: 15. In some implementations, a protein disclosed herein may have an amino acid sequence that consists of SEQ ID NO: 15. In some implementations, a protein of the present disclosure may consist of 808 amino acids that have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 15. In some implementations, a protein of the present disclosure may comprise more than 808 amino acids, but wherein about 808 of the total amino acids have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 15.

The present disclosure provides proteins that have at least about 65%—e.g., at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 16. In some implementations, a protein disclosed herein may have an amino acid sequence that comprises SEQ ID NO: 16. In some implementations, a protein disclosed herein may have an amino acid sequence that consists of SEQ ID NO: 16. In some implementations, a protein of the present disclosure may consist of 579 amino acids that have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 16. In some implementations, a protein of the present disclosure may comprise more than 579 amino acids, but wherein about 579 of the total amino acids have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 16.

In some implementations, the protein may be share at least about 90% identity with SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, or SEQ ID NO: 16. In some implementations, the protein may be share at least about 95% identity with SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, or SEQ ID NO: 16. In some implementations, the protein may be share at least about 99% identity with SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, or SEQ ID NO: 16. Thus, this disclosure contemplates and encompasses proteins with varying degrees of sequence identity compared to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, or SEQ ID NO: 16, so long as the protein exhibits HMGR activity, is able to produce mevalonate, or both.

HMGR enzymes may be structurally similar and comprise conserved regions or domains. As such, SEQ ID NOs: 1-16 and other HMGR enzymes within the scope of this disclosure may share a high degree of structural homology. Further, given the conversed regions and domains of HMGRs, the present disclosure contemplates HMGR enzyme variants in which the native inhibition domain has be removed or deleted (see, e.g., SEQ ID NOs: 1-6). Removal of the inhibitory domain (which is usually present only in animal-derived HMGR enzymes) may improve the activity level of the enzyme; and, because the conversion of HMG-CoA to mevalonate may often be rate limiting, such removal may improve the overall flux of isoprenoid production. Removal of the inhibitory domain may comprise a deletion of about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, about 300, about 305, about 310, about 315, about 320, about 325, about 330, about 335, about 340, about 345, about 350, about 355, about 360, about 365, about 370, about 375, about 380, about 385, about 390, about 395, or about 400 amino acids from the N-terminus of the protein.

Examples of HMGR enzymes from which the N-terminal inhibitory domain has been removed include the truncated proteins represented by SEQ ID NOs: 1 (tAgHMGR_543), 2 (tDmHMGR_390), 3 (tEcHMGR_466), 4 (tFfHMGR_610), 5 (tUnHMGR_487), and 6 (tHMGR_531). The removal of the N-terminal inhibitory domain in these proteins (i.e., SEQ ID NOs: 1-6) is based on sequence alignment to known structures/domains in HMGR enzymes. Nevertheless, in some implementations, further truncations may be possible while still maintaining HMGR activity. Thus, the present disclosure provides HMGR enzymes that are variants of any one of SEQ ID NOs: 1-6, in which up to 30, up to 25, up to 20, up to 15, up 14, up to 13, up to 12, up to 11, up to 10, up to 9, up to 8, up to 7, up to 6, up to 5, up to 4, up to 3, up to 2, or 1 amino acid(s) is/are removed from the N-terminus of any one of SEQ ID NOs: 1-6. Similarly, present disclosure provides HMGR enzymes that are variants of any one of SEQ ID NOs: 1-6, in which up to 30, up to 25, up to 20, up to 15, up 14, up to 13, up to 12, up to 11, up to 10, up to 9, up to 8, up to 7, up to 6, up to 5, up to 4, up to 3, up to 2, or 1 amino acid(s) is/are added to the N-terminus of any one of SEQ ID NOs: 1-6.

By the same token, the present disclosure likewise provides truncated variants of SEQ ID NOs: 7-16, wherein the N-terminal inhibitory domain has been removed or deleted. In such variants, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, about 300, about 305, about 310, about 315, about 320, about 325, about 330, about 335, about 340, about 345, about 350, about 355, about 360, about 365, about 370, about 375, about 380, about 385, about 390, about 395, or about 400 amino acids may be deleted from the N-terminus of any one of SEQ ID NOs: 7-16. For the purposes of such variants, HMGR activity should be maintained (i.e., the enzyme should be able to convert HMG-CoA to mevalonate).

For the purposes of this disclosure, all of the foregoing proteins or enzymes can be isolated and/or engineered in a form in which the protein is substantially free of other proteins, contaminants, or macromolecules (e.g., nucleic acids, lipids, etc.). However, it should be understood that an “isolated” protein or enzyme may not be 100% free of other proteins, contaminants, or macromolecules, and absolute purity is not required in order for a protein or enzyme to be considered “isolated.”

The present disclosure also provides nucleic acids comprising a nucleic acid sequence encoding any one of the proteins disclosed herein. Those skilled in the art understand that a nucleic acid sequence can be designed/determined based on a known amino acid sequence as a result of known codon specificity, and codon can be optimized based on the organism (e.g., yeast) that will be expressing the sequence. Thus, in some implementations, the nucleic acid may comprise a nucleic acid sequence encoding any one of SEQ ID NOs: 1-16, or a protein that has at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with any one of SEQ ID NOs: 1-16, so long as the encoded protein exhibits HMGR activity. Some exemplary nucleic acid sequences are disclosed in Table 2 below.

TABLE 2
Nucleic Acid Sequences of Certain
Disclosed HMGR Enzymes

| | | SEQ |
|---|---|---|
| | | ID |
| Name | Nucleic Acid Sequence | NO |
| tAgHMGR_ | ATGGCACGTGAAACTATTCCAAAATCTACG | 17 |
| 543 | GTTTCGTCTTCTGAAACTAAAGTGGTTGGC | |
| | TCTGTAGCCTCTTCAATCGTCCCAAGCGAC | |
| | GATGAAACCGAAACGGAAGATGAAGCGGAA | |
| | CCTGTCAGACCATTAGCAACTTTAATCGAC | |
| | GTCTTGAGGAAGGGGGCTGTGAAAACATTG | |
| | AAGAACAAAGAAGTTGTTTCTCTTGTTGTT | |
| | AACTCTGAACTACCACTATATGCTTTAGAA | |
| | AAGCAATTAGGAGACACGACTAGGGCAGTT | |
| | ATTGTCAGAAGAAAGGCTTTGGCAAAGTTA | |
| | GCTGATGCACCTGTTCTGGAAACGGAAAGA | |
| | TTGCCTTATAAACATTACGACTATGATAGA | |
| | GTCTTCGGTGCGTGTTGCGAAAACGTTATC | |
| | GGGTATATGCCATTGCCCGTTGGCGTAATT | |
| | GGCCCCTTAGTCATTGACGGTGTTGCCTAC | |
| | CATATCCCAATGGCAACTACTGAAGGTTGT | |
| | TTGGTCGCATCCGCTATGAGAGGATGTAAA | |
| | GCTATAAACGCAGGTGGAGGTGTAACCACT | |
| | GTCTTAACAAAGGATGGCATGACCAGAGGG | |
| | CCATGCGTCAGATTTCCATCGCTGGCGAGG | |
| | GCGGGTGCTTGTAAGTIGTGGCTAGATAGT | |
| | GAAGAGGGGCAGGCAAGAGTCAAACGTGCT | |
| | TTCAATTCCACCAGCAGATTCGCTCGTCTG | |
| | CAACACGTTCAGACGGCTTTAGCCGGTGAT | |
| | TTGTTGTTCATTAGGTTTAGGACTACAACT | |
| | GGAGACGCTATGGGGATGAACATGATTAGT | |
| | AAAGGTGTCGAATTTGCATTGCATCAAATG | |
| | GGCGCGGAATTTGGTTGGCATGATATGGAA | |
| | ATTGTTGCTGTTTCTGGAAATTATTGTACG | |
| | GATAAGAAACCCGCAGCCATAAATTGGATA | |
| | GAGGGTAGAGGTAAAAGCGTTGTCGCTGAG | |
| | GCAACCGTGCCTGCAGACGTCGTGAGAAAA | |
| | GTATTGAAGTCTGACGTCGCCGCATTGGTT | |
| | GATGTCAATATATCCAAAAATTTAGTAGGC | |
| | TCTGCAATGGCGGGAGCTGTTGGTGGTTTC | |
| | AACGCACACGCTAGTAACCTTGTCACAGCC | |
| | TTGTACCTGGCTCTAGGTCAAGACCCAGCC | |
| | CAAAATGTTGAAAGTTCCAATTGCATAACA | |
| | CTGATGAGGGATGTGGGTGGAGACCTAAGA | |
| | GTTTCGGTAAGCATGCCAAGTATAGAAGTT | |
| | GGGACCATCGGTGGAGGAACAATTTTAGGT | |
| | CCCCAGTCGGCAATGCTTGACTTATTAGGT | |
| | GTCAGAGGTCCTCATCCAAGCGCCCCTGGT | |
| | ACAAATGCAAGACAGTTGGCTAAAATTGTT | |
| | GCTTCTGCTGTTCTTGCTGGCGAACTTTCT | |
| | CTGTGTAGCGCCTTAGCTGCTGGACATCTA | |
| | GTCCAATCCCACATGATTCATAACAGAGCA | |
| | AAAACGCCAGCAGATCCGGAAGTTCCATGT | |
| | AGGAGACCCGCATGTATTTGA | |
| tDmHMGR_ | ATGGACCAGTTAAGGCAATCCGGGCCAGTT | 18 |
| 390 | GCGATAGCCGCTAAGGCTTCACAAACGACT | |
| | CCAATTGACGAGGAACATGTGGAACAAGAA | |
| | AAAGACACGGAAAATTCAGCGGCTGTTAGG | |
| | ACTTTGCTATTTACTATCGAAGATCAATCT | |
| | TCTGCAAATGCATCTACGCAAACTGATTTG | |
| | TTGCCCTTGCGTCATAGATTGGTCGGACCG | |
| | ATTAAACCACCTAGACCAGTACAAGAATGC | |
| | CTTGATATACTGAACAGTACTGAAGAGGGG | |
| | TCCGGGCCTGCGGCTCTTTCAGATGAAGAA | |
| | ATCGTATCAATTGTTCATGCTGGAGGAACT | |
| | CATTGTCCATTACATAAGATTGAATCAGTA | |
| | TTAGATGACCCGGAAAGGGGCGTGAGGATA | |
| | AGAAGGCAAATAATCGGTAGTCGTGCAAAA | |
| | ATGCCTGTTGGTAGACTAGATGTTCTACCA | |
| | TACGAACACTTCGACTATAGAAAGGTCTTA | |
| | AACGCCTGTTGCGAAAACGTATTGGGTTAC | |
| | GTGCCAATCCCAGTTGGTTATGCAGGTCCA | |
| | TTGTTATTAGATGGTGAAACATATTATGTA | |
| | CCTATGGCGACTACGGAGGGAGCTCTAGTT | |
| | GCATCCACTAATAGGGGTTGTAAGGCATTG | |
| | AGCGTAAGAGGAGTAAGATCCGTGGTCGAA | |
| | GATGTAGGCATGACCAGAGCACCCTGCGTC | |
| | AGGTTTCCTTCAGTAGCAAGAGCAGCTGAA | |
| | GCTAAGTCTTGGATAGAAAATGATGAAAAC | |
| | TATAGAGTAGTCAAAACAGAATTTGATTCT | |
| | ACCAGTAGATTTGGTCGTCTGAAAGATTGT | |
| | CACATAGCTATGGATGGGCCACAATTATAC | |
| | ATTAGATTCGTAGCTATTACTGGTGATGCA | |
| | ATGGGTATGAACATGGTGTCCAAAGGCGCT | |
| | GAAATGGCACTTAGAAGGATCCAATTGCAA | |
| | TTTCCGGATATGCAAATTATATCCTTGTCA | |
| | GGTAATTTTTGCTGCGATAAAAAGCCAGCC | |
| | GCAATAAATTGGATAAAGGGAAGAGGTAAA | |
| | AGGGTTGTTACTGAATGTACCATATCTGCA | |
| | GCAACTCTGAGGTCAGTCTTAAAGACTGAT | |
| | GCTAAAACGCTAGITGAATGTAATAAGTTA | |
| | AAAAATATGGGAGGCAGTGCTATGGCAGGA | |
| | TCAATAGGAGGAAACAATGCACATGCTGCA | |
| | AATATGGTTACTGCTGTATTTTTGGCTACA | |
| | GGTCAAGATCCTGCACAGAATGTTACTTCG | |
| | AGCAATTGCTCAACCGCTATGGAGTGTTGG | |
| | GCTGAAAATTCCGAAGATTTATACATGACT | |
| | TGTACCATGCCAAGCTTGGAAGTCGGTACG | |
| | GTAGGTGGAGGTACCGGCTTGCCAGGTCAG | |
| | AGTGCATGCCTAGAAATGCTGGGTGTACGT | |
| | GGCGCTCATGCGACTAGACCCGGTGATAAT | |
| | GCAAAAAAGTTGGCGCAAATCGTCTGCGCT | |
| | ACTGTTATGGCTGGAGAATTGAGTTTAATG | |
| | GCAGCACTGGTAAACTCTGATTTAGTTAAA | |
| | TCTCATATGAGACATAACAGATCTTCTATA | |
| | GCTGTGAACTCTGCTAATAATCCTCTTAAT | |
| | GTAACCGTCTCATCTTGTTCTACGATTTCT | |
| | TAA | |
| tEcHMGR_ | ATGAAGGTCTATTTTCAATTAGAGGATACT | 19 |
| 466 | ATCCTTTCGAGTCTAAGGTATGTGTCGGTT | |
| | GCCATCAGAGATAGATTCATTAGTAAATTA | |
| | GTACTTTTTGCTCTAGCTATCAGTGCGTCT | |
| | ATCAACATATATCTGTTGAACATAGCTAGA | |
| | ATTCACACTCAATTCACGACCAATGAACTT | |
| | AATAGTAAGAAAAAATTGAAGAAATCTAGT | |
| | AATTTCGCAGTTGGCAGTGCTCCAATAGTT | |
| | GCTCCACCATCGGAAAGAACCAGCGAATCT | |
| | ACAGTATCATCCTCAGAAACTAAAATAATG | |
| | GACTCAGTCCCTTCGTCTGTCACCGTCTCT | |
| | GATGACGAAACTGAAACTGAAGATGAATCC | |
| | GAACCCATTAGACCGTTGGAAACTTTAATT | |
| | GAAATTATGAAACAGGGTGGAGTTAAAACT | |
| | TTGAGAAACAGAGAATTAGTCTCCTTGATC | |
| | GTTAATAGTGAACTGCCCCTGTACGCATTA | |
| | GAAAAGCAACTATGTGATACCACAAGGGCC | |
| | GTCGTTGTTAGAAGAAAAGCATTGGCCAAA | |
| | CTTGCAGATGCTCCAGCGTTAGAAACTGAA | |
| | CGTCTACCATACAAAAATTATGACTATGAT | |
| | AGGGTTTTTGGAGCTTGCTGTGAAAATGTT | |
| | ATCGGTTATATGCCCCTTCCCGTTGGGGTC | |
| | ATTGGACCTCTGGTTATCGATGGTATACCT | |
| | TATCACATACCAATGGCAACTACAGAAGGT | |
| | TGTTTAGTAGCCTCGGCGATGAGAGGTTGC | |
| | AAGGCAATAAATGCCGGAGGAGGGGTGACA | |
| | ACAGTATTGACTAAGGATGGAATGACAAGG | |
| | GGTCCATGCATTAGATTCCCTTCTTTGGCA | |
| | AGATCAGGTGCTTGTAAGATTTGGTTAGAC | |
| | TCTGAGGATGGTCAAAATAAAATAAAGAAA | |
| | GCATTTAACTCTACATCCAGATTTGCTCGT | |
| | CTACAGCATATTCAAACTGCGTTAGCGGGC | |
| | GATCTTTTATTCATTAGATTCAGAACCACT | |
| | ACGGGTGATGCGATGGGTATGAATATGATC | |
| | TCCAAGGGCGTAGAATTCAGCCTTAATCAG | |
| | ATGGTGGAGGAGTTCGGGTGGGACGACATG | |
| | GAAATAGTAGCCGTGTCCGGTAACTATTGC | |
| | ACAGACAAAAAGCCAGCGGCAATCAATTGG | |
| | ATTGAAGGAAGAGGTAAATCAGTGGTAGCT | |
| | GAGGCAACTATTCCAGGGGATGTCGTAAAG | |
| | AAAGTTCTTAAGTCTGACGTCAATGCTCTA | |
| | GTCGACCTTAATATTAGCAAAAATTTAATC | |
| | GGTAGTGCTATGGCTGGTAGTGTTGGGGGA | |
| | TTTAATGCTCATGCCTCCAACTTAGTCACA | |
| | GCAGTTTACCTAGCTCTAGGACAGGATCCA | |
| | GCCCAAAATGTTGAATCCTCTAACTGTATG | |
| | ACATTAATGAAAGAAATTGACGGTGACTTA | |
| | AGAATAAGCGTCAGTATGCCTTCTATAGAA | |
| | GTTGGTACGATCGGTGGTGGTACAATATTG | |
| | GAACCCCAGTCAGCTATGTTGGATTTATTG | |
| | GGAGTGAGAGGCCCCCATCCTACTGAGCCT | |
| | GGCACCAACGCAAGGCAATTAGCTAAAGTT | |
| | GTGGCCTGCGCCGTCATGGCCGGAGAATTA | |
| | TCCCTTTGTTCTGCTCTAGCAGCTGGTCAT | |
| | CTAGTGCAGAGCCACATGATCCATAATAGA | |
| | GCCAAAGCTTCACCTACCTCCACCGAGGTT | |
| | AAGCAAGACGATATTCCACGTTTGCAAGAA | |
| | GGCTCTGTTACGTGTATAAAGTAA | |
| tFfHMGR_ | ATGGTCTTGTCAAAATGGATTGTTATCGCT | 20 |
| 610 | CTGGCACTTAGTGTCGCACTGAATGGATAC | |
| | TTATTTAATGTGGCTCGTTGGGGTATTAAA | |
| | GATCCAAATGTACCAGAACATAATATTGAT | |
| | AGGAATGAATTAGCGCGTGCACAACAATTT | |
| | AACGACACAGGCTCCGCAACTTTGCCCTTA | |
| | GGTGAGTATGTCCCTCCTACGCCAATGAGG | |
| | ACACAGCCATCGACTCCAGCTATAACCGAC | |
| | GACGAAGCAGAGGGTTTACATATGACTAAG | |
| | GCGAGACCTGCTAATTTACCCAATAGATCA | |
| | AATGAAGAATTAGAAAAGTTGCTTTCAGAA | |
| | AAGAGAGTGAGAGAAATGACAGACGAAGAA | |
| | GTAATCTCATTATCCATGAGAGGGAAGATA | |
| | CCAGGTTACGCATTAGAAAAGACACTAGGT | |
| | GATTTCACAAGAGCTGTGAAAATCAGACGT | |
| | TCAATAATAGCTAGGAATAAAGCAACGACG | |
| | GATATTACACACTCATTGGACAGGAGTAAG | |
| | TTGCCTTATGAGAATTACAATTGGGAAAGA | |
| | GTTTTCGGTGCATGTTGCGAAAACGTCATC | |
| | GGTTATATGCCCTTGCCAGTCGGTGTCGCT | |
| | GGCCCTTTAGTAATTGATGGACAATCGTAT | |
| | TTCATTCCTATGGCGACCACTGAAGGTGTT | |
| | TTGGTAGCTTCGGCCAGTAGAGGATGCAAG | |
| | GCAATTAATTCCGGCGGTGGGGCCATCACT | |
| | GTATTGACGGCGGATGGGATGACTAGGGGA | |
| | CCTTGTGTAGCCTTTGAAACGTTGGAAAGA | |
| | GCTGGTGCAGCAAAGCTTTGGTTGGACTCA | |
| | GAAGCCGGGCAAGATATGATGAAGAAGGCG | |
| | TTCAATTCTACATCCAGATTTGCTAGATTG | |
| | CAATCTATGAAAACTGCCCTTGCAGGCACA | |
| | AATTTATATATAAGATTTAAGACAACCACA | |
| | GGTGACGCAATGGGAATGAATATGATTAGC | |
| | AAAGGAGTTGAACACGCTTTGTCTGTCATG | |
| | GCTAATGACGGTGGTTTCGACGACATGCAA | |
| | ATAATCTCCGTCTCAGGCAATTACTGTACG | |
| | GATAAGAAAGCTGCTGCGCTAAATTGGATA | |
| | GACGGAAGAGGCAAAGGTGTCGTTGCTGAG | |
| | GCTATAATCCCTGGCGAGGTGGTGAGATCT | |
| | GTGCTTAAATCCGATGTGGACTCGCTAGTA | |
| | GAATTGAATGTCGCAAAAAACTTGATCGGT | |
| | TCAGCAATGGCCGGCTCTGTGGGCGGATTC | |
| | AATGCGCATGCAGCTAACATAGTGGCAGCC | |
| | ATATTCCTGGCCACGGGGCAGGACCCTGCT | |
| | CAAGTGGTAGAGTCTGCTAATTGCATAACG | |
| | ATTATGAAAAACTTAAACGGTGCGCTACAA | |
| | ATCTCAGTCTCTATGCCCAGCTTGGAAGTT | |
| | GGCACTCTGGGTGGAGGTACTATATTGGAA | |
| | CCGCAAGGTGCTATGTTGGATATACTTGGA | |
| | GTAAGAGGTTCTCATCCCACTAACCCGGGT | |
| | GATAATGCTCGTAGACTTGCAAGAATAATT | |
| | GGCGCGGCTGTACTAGCTGGTGAGTTGTCC | |
| | CTGTGCTCCGCTTTGGCTGCTGGACATCTT | |
| | GTAAGAGCGCACATGCAGCATAATAGGTCT | |
| | GCAGCTCCCAGTAGATCTACTACTCCCGCT | |
| | CCTCCAATGACACCTGTTTCTTTAGCAATG | |
| | ACCAGCGCACAGGAAAGATCAGCTAGTACA | |
| | ACGTCTATGTCAGCAGCTGCAATTCAGAGG | |
| | TCTAAGTAA | |
| tUnHMGR_ | ATGAGTACAATAAAGTACCACCAAAACGAG | 21 |
| 487 | GCAAGTGACGTTATATCTAGTTCGCCTCAG | |
| | AAGAAATCTCAATTCATGGAAACTAATCAA | |
| | CCTTATGACAATACTTTGCAAACCCCTATT | |
| | AACATCGATGATGAAGAAGAAGGTCTAGAG | |
| | ATCTGTTTGAGCAAGTCAAAGTCACCATCA | |
| | AAAAGGACTCAAGCGCAACTTGAAATGATG | |
| | TTAAAAGAGAATCAAGCCAGCGAATTGGAT | |
| | GATCAGGAATTGATTGAATTATCATTACAG | |
| | GGCAAGATTCCTGGGTACGCATTAGAGAAG | |
| | AAGTTAAAAGATACAACTAGAGCGGTTAAA | |
| | ATAAGACGTGCGGTTATTTCTCGTACGTTA | |
| | ACAACTTCTCAGACGACTGGGTTATTAGAG | |
| | TACTCGAAATTACCTTATAAGAACTATGAT | |
| | TGGGACAGAGTTTTAGGGGCGTGTTGTGAA | |
| | AATGTTATAGGTTACATGCCATTGCCATTA | |
| | GGTGTAGCGGGTCCAATAATTATTGATTCA | |
| | CAATCTTATTTTATCCCTATGGCAACGACC | |
| | GAAGGTGTACTTGTGGCTTCCACTTCTAGA | |
| | GGCGCTAAGGCCATTAACGCAGGGGGAGGA | |
| | GCTGTTACGGTCATAACCGGTGATGGCATG | |
| | ACCAGAGGTCCTTGCGTTTCTTTTGAAACA | |
| | TTGGAACGTGCTGGAGCGGCTAAAGTATGG | |
| | CTTGATAGTGAAATTGGCCAAAAAATCATA | |
| | ACGAAAGCTTTTAACTCCACGTCCAGATTT | |
| | GCTAGGTTACAATCAATCAAGACGGCATTA | |
| | GCCGGTACTTATTTATATCCGAGGTTTAAA | |
| | ACCACCACTGGTGATGCAATGGGTATGAAC | |
| | ATGATCTCTAAAGGTGTCGAACATGCGTTG | |
| | AATGTAATGGCCACTGAAGCTGGTTTTGAA | |
| | GACATGCAGATCATCTCAGTAAGTGGTAAT | |
| | TTTTGTACCGATAAAAAGCCAGCTGCTATC | |
| | AATTGGATCGACGGTAGGGGTAAATCGGTT | |
| | GTTGCAGAGGCGATCATTCCTAAAGATATT | |
| | GTCAAATCGGTGTTGAAGTCAACCGTAGAT | |
| | GCCATGGTTGAATTAAATATTTCTAAAAAT | |
| | TTGGTGGGCTCTGCAATGGCCGGCTCTATA | |
| | GGTGGTTTTAATGCCCATGCCGCCAATATA | |
| | GTTACCGCTATTTTTTTAGCCACTGGGCAG | |
| | GACCCTGCTCAAAACGTTGAATCCAGCAAC | |
| | TGTATTACCTTGATGAGGAATTTGGGTGGT | |
| | AACCTGCAAATTTCCGTGTCTATGCCATCC | |
| | ATTGAGGTTGGTACTCTGGGTGGTGGAACA | |
| | ATTTTGGAACCACAAGGCGCAATGTTGGAC | |
| | ATGTTGGGTGTAAGGGGTTCACATCCCACA | |
| | CACCCTGGAGAAAATGCTCGTAGATTGGCT | |
| | AGGATTATTGCCGCTTCTGTTTTAAGTGGA | |
| | GAATTAAGCCTATGCTCCGCCCTAGCTGCT | |
| | GGTCACTTGGTAAAATCACACATGGCACAC | |
| | AACAGGTCTGCACCTATAACCAGAAGTAAT | |
| | ACCCCTGCACAAATTTCGACACACCCAAGT | |
| | ATGATCAGTACCAACTCCATGAGGGAAAAA | |
| | CATTAA | |
| tHMGR_ | ATGGCCGCTGACCAACTGGTGAAAACCGAA | 22 |
| 531 | GTTACCAAAAAGTCTTTTACTGCACCTGTT | |
| | CAAAAAGCTAGCACGCCTGTGTTGACCAAC | |
| | AAGACTGTAATATCCGGGTCCAAGGTAAAA | |
| | TCACTAAGTTCTGCACAGTCCTCTAGCTCC | |
| | GGTCCGAGTTCTTCTTCAGAAGAAGATGAT | |
| | TCGAGGGATATTGAATCACTTGATAAGAAA | |
| | ATAAGGCCTTTGGAAGAACTGGAGGCACTA | |
| | TTGTCTAGCGGTAATACCAAGCAATTAAAA | |
| | AATAAAGAAGTTGCCGCTCTAGTTATTCAC | |
| | GGTAAATTACCGTTATACGCTTTGGAAAAA | |
| | AAGTTAGGTGACACCACTCGTGCCGTCGCT | |
| | GTCAGAAGGAAAGCACTATCAATCCTGGCA | |
| | GAAGCTCCAGTGCTAGCTTCCGACAGGCTG | |
| | CCTTATAAAAATTACGATTATGACAGGGTT | |
| | TTCGGTGCATGTTGCGAAAACGTAATCGGA | |
| | TATATGCCGCTGCCTGTTGGTGTCATAGGA | |
| | CCCCTTGTTATCGATGGCACCTCATACCAT | |
| | ATCCCAATGGCTACTACGGAGGGTTGTTTA | |
| | GTTGCAAGTGCTATGAGAGGGTGTAAGGCC | |
| | ATCAATGCCGGAGGTGGTGCGACTACTGTG | |
| | CTGACTAAGGATGGTATGACTAGAGGCCCT | |
| | GTAGTTAGATTTCCCACTCTAAAGAGAAGT | |
| | GGTGCTTGTAAGATCTGGTTGGACTCAGAG | |
| | GAAGGTCAAAACGCTATCAAAAAAGCATTT | |
| | AACAGCACATCTAGATTCGCTAGATTACAG | |
| | CACATTCAAACATGCTTGGCAGGCGATCTT | |
| | CTGTTTATGAGGTTCCGTACAACTACCGGC | |
| | GATGCAATGGGAATGAACATGATTTCTAAG | |
| | GGTGTCGAATATTCACTGAAACAGATGGTT | |
| | GAGGAATACGGCTGGGAAGACATGGAAGTG | |
| | GTCTCAGTCTCAGGAAACTACTGCACAGAT | |
| | AAAAAGCCAGCTGCAATCAATTGGATTGAA | |
| | GGAAGAGGTAAATCCGTCGTTGCAGAAGCT | |
| | ACCATTCCTGGTGACGTGGTTAGAAAAGTT | |
| | CTTAAGAGCGATGTATCTGCCCTTGTAGAA | |
| | TTAAACATTGCCAAGAATCTAGTCGGAAGT | |
| | GCAATGGCAGGCTCTGTTGGTGGCTTTAAT | |
| | GCACATGCAGCAAATTTAGTCACCGCGGTG | |
| | TTCCTGGCCCTTGGACAAGACCCAGCTCAA | |
| | AACGTTGAAAGTTCAAATTGCATAACTTTG | |
| | ATGAAGGAAGTGGATGGTGATCTAAGAATT | |
| | TCCGTGTCAATGCCATCCATTGAAGTCGGT | |
| | ACCATCGGGGGAGGCACGGTTCTTGAGCCT | |
| | CAAGGTGCTATGTTAGATCTTTTAGGTGTA | |
| | AGAGGCCCACATGCGACCGCGCCTGGTACA | |
| | AACGCTAGACAATTGGCAAGAATTGTTGCC | |
| | TGTGCGGTGTTGGCAGGGGAATTAAGTTTG | |
| | TGTGCTGCTTTAGCTGCAGGTCACCTTGTT | |
| | CAATCCCATATGACTCATAATAGAAAACCA | |
| | GCCGAACCGACCAAACCTAACAATTTAGAC | |
| | GCAACTGACATAAATAGACTTAAGGATGGA | |
| | TCAGTTACTTGTATTAAATCATGA | |
| EfMvaE | ATGAAGACCGTCGTGATAATTGACGCCTTG | 23 |
| | AGAACTCCAATAGGTAAGTATAAGGGTTCT | |
| | CTGTCTCAAGTTTCTGCTGTTGATTTAGGA | |
| | ACACATGTAACCACCCAGTTGCTGAAGAGG | |
| | CATTCTACCATTAGTGAAGAGATAGATCAA | |
| | GTCATATTTGGTAACGTGCTTCAAGCTGGT | |
| | AATGGTCAAAATCCGGCAAGACAAATTGCC | |
| | ATTAACTCAGGTCTGTCACATGAAATACCA | |
| | GCTATGACTGTAAATGAGGTGTGTGGCTCA | |
| | GGCATGAAGGCGGTCATTCTTGCTAAGCAA | |
| | CTGATTCAGCTGGGTGAAGCAGAAGTTTTG | |
| | ATAGCAGGTGGAATTGAAAACATGTCACAA | |
| | GCACCAAAATTGCAAAGGTTTAACTACGAG | |
| | ACTGAGAGCTATGATGCACCTTTCTCGTCC | |
| | ATGATGTACGATGGTCTAACCGATGCGTTT | |
| | TCCGGACAAGCAATGGGTTTAACAGCTGAG | |
| | AATGTTGCAGAAAAGTATCATGTCACAAGG | |
| | GAGGAACAGGATCAATTCTCTGTCCACTCT | |
| | CAACTTAAAGCAGCTCAAGCTCAGGCTGAA | |
| | GGCATATTTGCGGACGAAATTGCACCGCTT | |
| | GAAGTCTCAGGCACTTTAGTCGAAAAGGAT | |
| | GAGGGCATAAGACCAAACTCTTCTGTTGAG | |
| | AAACTGGGAACCCTAAAAACAGTGTTTAAA | |
| | GAAGACGGTACAGTCACTGCAGGAAATGCC | |
| | AGCACTATTAATGACGGGGCCAGCGCCCTA | |
| | ATTATAGCTTCACAAGAATATGCAGAAGCC | |
| | CATGGACTTCCCTATCTGGCGATTATTAGA | |
| | GATAGCGTTGAGGTCGGCATTGATCCTGCT | |
| | TATATGGGCATCTCGCCAATCAAAGCTATC | |
| | CAGAAGCTATTAGCACGTAACCAATTGACC | |
| | ACAGAAGAAATTGACTTATATGAGATCAAC | |
| | GAAGCATTTGCCGCAACTAGCATAGTGGTG | |
| | CAGAGAGAGCTGGCGTTACCAGAGGAGAAG | |
| | GTTAACATATACGGCGGTGGCATCTCGCTT | |
| | GGTCATGCAATTGGTGCTACCGGTGCAAGG | |
| | TTATTAACCTCTCTTAGTTACCAATTGAAC | |
| | CAAAAAGAAAAGAAGTACGGTGTCGCATCA | |
| | CTTTGTATAGGTGGAGGTTTGGGTTTAGCA | |
| | ATGTTGTTAGAACGTCCCCAGCAGAAAAAG | |
| | AACTCCAGATTTTATCAAATGTCTCCAGAA | |
| | GAAAGATTGGCGTCTTTGTTGAACGAAGGT | |
| | CAAATTAGTGCAGATACAAAAAAAGAGTTC | |
| | GAAAACACAGCTCTATCTAGCCAAATAGCT | |
| | AATCACATGATTGAAAATCAGATTTCTGAA | |
| | ACAGAAGTACCTATGGGCGTTGGTTTACAT | |
| | TTAACTGTAGACGAAACCGACTATTTAGTC | |
| | CCTATGGCTACTGAGGAACCATCAGTCATA | |
| | GCGGCCCTGAGTAATGGTGCGAAAATCGCT | |
| | CAGGGTTTTAAGACTGTGAACCAACAAAGA | |
| | TTGATGAGAGGTCAAATTGTTTTTTATGAC | |
| | GTGGCTGATGCCGAATCTTTAATTGATGAG | |
| | TTGCAGGTCAGAGAAACAGAAATTTTTCAA | |
| | CAAGCCGAATTATCTTATCCTTCAATTGTT | |
| | AAAAGAGGTGGCGGCTTGAGAGATTTACAA | |
| | TACCGTGCCTTCGATGAATCATTCGTATCA | |
| | GTTGATTTCCTAGTCGATGTTAAGGACGCC | |
| | ATGGGTGCTAATATAGTTAATGCTATGCTA | |
| | GAGGGCGTAGCTGAGTTATTCAGAGAATGG | |
| | TTTGCTGAGCAAAAAATACTATTTTCCATA | |
| | CTAAGTAATTATGCAACGGAGTCAGTAGTA | |
| | ACCATGAAGACAGCTATTCCTGTATCGAGA | |
| | TTAAGCAAGGGGAGTAACGGTCGTGAGATT | |
| | GCAGAAAAGATTGTCCTAGCTAGCAGGTAC | |
| | GCTTCCCTAGATCCATACAGAGCTGTTACA | |
| | CATAATAAAGGCATCATGAATGGTATTGAG | |
| | GCCGTTGTCCTAGCCACAGGAAATGATACG | |
| | AGAGCTGTTTCTGCCTCTTGTCATGCTTTC | |
| | GCAGTTAAAGAGGGTAGGTACCAAGGTTTG | |
| | ACTAGTTGGACTCTGGATGGTGAACAACTA | |
| | ATTGGTGAAATTAGTGTTCCATTGGCCTTA | |
| | GCTACTGTAGGAGGCGCTACCAAGGTACTG | |
| | CCTAAAAGCCAAGCTGCAGCAGACTTGCTT | |
| | GCTGTCACTGATGCCAAGGAATTGTCTAGA | |
| | GTGGTGGCTGCTGTGGGTCTAGCGCAAAAT | |
| | TTGGCTGCTCTACGTGCCTTGGTGAGTGAA | |
| | GGTATTCAAAAAGGACATATGGCTTTGCAA | |
| | GCTAGGTCTCTAGCTATGACAGTGGGTGCA | |
| | ACTGGCAAAGAAGTAGAAGCTGTCGCACAA | |
| | CAGCTTAAGAGACAAAAAACCATGAATCAA | |
| | GATAGGGCTTTAGCCATACTTAACGATTTG | |
| | AGAAAACAATGA | |
| DaHMGR | ATGGTTGCGGATTCTAGATTACCAAATTTT | 24 |
| | AGGGCACTAACTCCTGCGCAAAGGAGAGAC | |
| | TTCCTTGCAGATGCATGTGGACTTTCCGAT | |
| | GCAGAAAGGGCATTGCTGGCCGCCCCAGGT | |
| | GCATTGCCCCTAGCTCTAGCAGATGGCATG | |
| | ATTGAAAACGTATTTGGATCCTTTGAACTG | |
| | CCATTAGGAGTAGCGGGGAACTTTAGAGTT | |
| | AATGGTCGTGATGTGCTTGTACCAATGGCA | |
| | GTTGAAGAACCCAGTGTTGTGGCTGCCGCC | |
| | TCTTACATGGCTAAATTGGCTAGAGAGGAT | |
| | GGTGGTTTCCAAACAAGTTCAACACTACCG | |
| | TTAATGAGAGCACAAGTTCAAGTCCTTGGT | |
| | GTTACAGATCCCCATGGTGCTAGATTAGCC | |
| | GTCTTACAGGCACGTGCACAAATAATTGAA | |
| | AGAGCCAATTCTAGAGATAAAGTTTTGATC | |
| | GGATTAGGCGGTGGTTGTAAGGACATTGAA | |
| | GTGCATGTGTTTCCGGACACTCCAAGGGGA | |
| | CCAATGTTGGTGGTTCACCTAATTGTCGAT | |
| | GTTAGGGATGCGATGGGTGCAAATACGGTA | |
| | AACACGATGGCTGAGTCGGTAGCACCTTTG | |
| | GTTGAAAAAATAACCGGTGGATCCGTTAGA | |
| | CTGAGAATATTAAGTAACTTGGCTGACCTA | |
| | CGTTTGGCCAGGGCTAGGGTTAGATTAACC | |
| | CCACAAACTCTAGCGACTCAGGATAGATCC | |
| | GGAGAAGAAATTATCGAGGGTGTTCTTGAC | |
| | GCTTATACTTTTGCCGCCATTGACCCCTAC | |
| | CGTGCTGCCACCCATAACAAAGGTATTATG | |
| | AATGGGATAGATCCTGTTATCGTTGCCACT | |
| | GGTAATGACTGGAGAGCGGTAGAAGCTGGT | |
| | GCTCACGCTTATGCTTCAAGATCTGGAAGC | |
| | TACACTAGCTTAACTAGATGGGAAAAAGAT | |
| | GCAGGTGGTGCATTGGTAGGTTCAATTGAA | |
| | TTACCCATGCCTGTCGGTCTGGTGGGTGGT | |
| | GCCACTAAGACGCATCCACTAGCAAGACTG | |
| | GCACTTAAAATAATGGACTTGCAAAGTGCT | |
| | CAGCAATTGGGTGAAATCGCAGCAGCTGTT | |
| | GGTCTAGCTCAAAACTTGGGTGCCTTAAGA | |
| | GCACTTGCGACGGAGGGGATCCAACGTGGT | |
| | CATATGGCCTTACATGCCAGAAATATAGCA | |
| | CTGGTGGCCGGTGCTACAGGTGATGAAGTT | |
| | GATGCCGTAGCCCGTCAATTAGCCGCAGAA | |
| | CATGATGTGAGAACAGATAGGGCCCTTGAG | |
| | GTTTTAGCCGCTCTAAGGGCCAGAGCATAA | |
| HvHMGR | ATGACTGATGCCGCCTCTCTGGCTGACAGG | 25 |
| | GTCAGAGAAGGTGACTTGCGTTTACATGAA | |
| | TTGGAAGCACACGCTGACGCAGATACTGCT | |
| | GCTGAAGCAAGGAGATTATTAGTAGAATCT | |
| | CAAAGTGGCGCATCGTTGGATGCAGTAGGA | |
| | AATTACGGTTTCCCCGCTGAAGCTGCCGAG | |
| | AGTGCAATCGAAAATATGGTTGGTTCGATT | |
| | CAAGTTCCTATGGGCGTTGCTGGTCCAGTT | |
| | TCCGTTGACGGCGGTTCGGTTGCGGGAGAA | |
| | AAATACTTGCCTCTTGCAACTACCGAAGGT | |
| | GCTTTACTTGCTAGTGTTAACAGGGGGTGC | |
| | TCAGTGATAAATAGCGCTGGCGGTGCTACA | |
| | GCAAGGGTTTTAAAAAGTGGTATGACCAGA | |
| | GCACCGGTCTTTAGAGTAGCTGATGTCGCG | |
| | GAGGCGGAAGCACTTGTTAGTTGGACAAGG | |
| | GACAATTTTGCAGCCCTTAAAGAGGCTGCG | |
| | GAAGAAACAACAAATCATGGAGAATTGCTG | |
| | GACGTAACACCTTACGTGGTCGGTAATTCA | |
| | GTCTATTTGAGATTCAGATATGATACCAAG | |
| | GATGCTATGGGTATGAACATGGCTACAATC | |
| | GCAACTGAAGCTGTGTGTGGTGTTGTTGAA | |
| | GCAGAAACGGCTGCCAGCCTTGTTGCATTA | |
| | TCAGGCAACTTGTGTTCAGACAAGAAACCT | |
| | GCTGCTATTAATGCTGTAGAAGGTCGTGGT | |
| | AGAAGCGTTACCGCTGATGTAAGGATTCCA | |
| | AGGGAAGTGGTCGAGGAAAGATTGCACACG | |
| | ACCCCTGAAGCTGTTGCCGAATTGAACACC | |
| | AGAAAAAATCTAGTCGGTTCTGCTAAGGCT | |
| | GCATCATTAGGCTTTAATGCTCATGTCGCT | |
| | AATGTTGTAGCCGCTATGTTTCTTGCGACA | |
| | GGTCAGGATGAAGCGCAGGTTGTTGAGGGT | |
| | GCAAACGCAATCACTACGGCTGAGGTACAA | |
| | GACGGAGATTTGTACGTATCAGTTTCCATC | |
| | GCGTCGTTAGAAGTAGGTACTGTAGGTGGT | |
| | GGTACCAAGTTGCCTACTCAATCTGAAGGT | |
| | TTAGACATATTAGGAGTCTCCGGAGGTGGG | |
| | GATCCTGCCGGTTCGAATGCTGACGCCTTA | |
| | GCTGAATGTATCGCTGTTGGTTCATTAGCA | |
| | GGTGAATTATCACTATTGTCGGCTTTAGCC | |
| | TCTCGTCATCTATCTTCTGCGCATGCAGAG | |
| | TTGGGCAGATGA | |
| LkMvaE | ATGAAGGAAGTAGTTATAATAGACGCCGCC | 26 |
| | AGAACCCCAATTGGTAAGTATAAGGGTTCT | |
| | TTAAGTTCGTTCTCTGCAGTGGAGTTAGGT | |
| | ACCATGGTTACAAAAAAATTATTAGAAAAA | |
| | GCAAGTATTAAGAAAGATGAAATTAACCAA | |
| | GTGATATTCGGCAACGTCTTACAAGCAGGA | |
| | AATGGGCAGAACGTCGCCAGGCAGATCTCT | |
| | ATTATATCTGACATTCCCGTTGATGTTCCT | |
| | GCCATGACTATTAATGAGGTGTGTGGGTCA | |
| | GGTATGAAGGCTGTCATTTTGGCAAGACAG | |
| | CTGATTCAACTGGGTGAGGCCGATTTAGTA | |
| | ATTGCCGGGGGTACAGAATCTATGACGCGT | |
| | GCTCCCTTATTACAACAGTTCGATTCTGAG | |
| | ACTACTAGCTATAACGGACCAATATCCTCT | |
| | ATGGTGAATGATGGGTTGACAGACACTTTC | |
| | AGTAACACGCACATGGGTTTGACCGCGGAG | |
| | AATGTCGCGGAACAATTCGGGGTTACCAGA | |
| | AAGGAACAAGACCAATACGCCTTAGATTCC | |
| | CAATTAAAGGCCGCTAAAGCAACAGAGAAT | |
| | AATGTCTTTAAAGAGGAAATTATTCCAGTC | |
| | ACTCTACCTGACGGAACGTTATTAGAGAAT | |
| | GACGAAGCCATTAGGGGCAACTCCTCATTA | |
| | GAAAAACTGGGAACTTTAAAAACAGTTTTC | |
| | TCTGAAAATGGTACTGTGACTGCAGGGAAC | |
| | GCATCTCCGCTGAATGATGGCGCCAGTGTT | |
| | ATGATTCTGGCTTCTAAGGAATATGCACTA | |
| | AAAAATGATTTACCCTACCTGGCCACCATA | |
| | AAGGGAGTAGCCGAAATAGGGATAGACCCA | |
| | TCAATTATGGGAATAGCCCCTATTAACGCA | |
| | ATCAATAGTCTACTGGAAAAAACAGATGTT | |
| | TCTTTGGATGCCATAGATCGTTTTGAAATT | |
| | AACGAAGCATTCGCGGCATCATCCATCGTA | |
| | GTTAATAGGGAACTACAACTTGACCCAGAA | |
| | AAGGTGAATAGTGATGGTGGTGCCATAGCA | |
| | CTTGGACATCCTATCGGTGCAAGTGGTGCA | |
| | AGAATTCTGACAACCTTGTCGTATGGGTTG | |
| | CAAAGAAACGAACAAAAATACGGCATTGCC | |
| | TCTCTATGTATCGGCGGAGGCTTAGGCCTT | |
| | GCAGTATTGCTTGAGGCGAATCAGGAAAAA | |
| | GCAGGCTCATTTAATGAGAAGAAAAAATTC | |
| | TATCAGCTAACTCCAGAGGAAAGAAGATCC | |
| | CAATTAGTCAGAGGGGGTGTAATCTCTAAG | |
| | GAATCAGCCGATCAGCTAAAAAATGAAAGG | |
| | TTGTCTGAAGACATCGCTAACCATCTTATT | |
| | GAGAACCAAATCTCGCAGGTCGAGATTCCG | |
| | ATGGGGGTCGCACAGAATTTTCAAATCAAT | |
| | GGAGAAAAGAAATGGGTGCCAATGGCTACC | |
| | GAAGAACCCTCTGTTATTGCCGCTGCAAGT | |
| | AATGGGGCTAAAATTTGCGGCAACATTACA | |
| | GCGAAGACCCCGCAAAGGCTAATGAGAGGA | |
| | CAAATCGTGCTAACTGGGAAGTCTGAATAT | |
| | CAAGCTATTATTGAAGCTATTGATACAAGG | |
| | AAGGATGAACTATTTCTTTGTGCTAATAAT | |
| | AGCTACCCTTCCATAGTAAAAAGGGGCGGT | |
| | GGTGTAAGAGACATCTCCACAAGAGAATTT | |
| | ATGGGATCTGATCATGCTTACGTATCGATT | |
| | GACTTCCTAATCGACGTAAAGGATGCTATG | |
| | GGAGCCAATATTGTCAACGCAATTTTGGAG | |
| | GGTGTTGCATCTCAATTAAGATCTTGGTTT | |
| | CCAGATGAAGAGATTTTGTTCTCAATTTTG | |
| | TCGAATCTAGCTACCGAATCCTTGGCTACT | |
| | GCGTGTTGTACCATTCCTTTTGAATACTTG | |
| | GGTAAGAGCAAGGAAGCTGGTAGACAAGTT | |
| | GCAGAAAAGATTCAGCAAGCCGCTGAATAT | |
| | GCTAAATTGGATGTTTATAGAGCCGCTACT | |
| | CATAATAAGGGCATTATGAATGGTATAGAA | |
| | GCTGTAATCTTAGCTACTGGTAATGATACA | |
| | AGAGCTGCTTCTGCCGCTATTCATGCATAT | |
| | GCGTCTAGAAATGGGTTCTATCAGGGCCTG | |
| | ACTGATTGGAAGATCGTTGACGGCCAGCTT | |
| | GTTGGGAAGTTAACAGTACCGCTAGCAGTA | |
| | GCAACAGTCGGTGGTGCTTCTAAAATTTTA | |
| | CCGAAGGCGAAATTGGCTCTTGAAATACTA | |
| | GACGTGTCATCTGCAAAGGAGCTAGCTCAA | |
| | GTGATCGCAGCTGTTGGTTTAGCACAAAAT | |
| | TTGGCTGCGTTGAAGGCCCTTGTCACAGAA | |
| | GGTATCCAAAAGGGTCATATGTCCTTACAG | |
| | GCTAGGGCTTTGGCTATTACCGTAGGTGCT | |
| | ACAGGCGATGAAATTGAACAGGTTGCGTCC | |
| | TACCTTAGAAAGGCCGATACCATGAACCAG | |
| | CAGTTAGCTTCCGACTATCTGCTGGAAACT | |
| | AGGAGCTAA | |
| MbHMGR | ATGGCTTCTAAAACTGAAACGACTATGAAG | 27 |
| | GAAGACGAATTGCTGGAGAAGGTCGTGAGT | |
| | GGTGAAATGCCACTTAGAAAAATTGATGCC | |
| | TACACAGATACTGATACAGCAGTGAGAGTA | |
| | AGGAAATGCGCAATAGAAAAAATGAATGGA | |
| | GTGAAGTTCGAACACATTCAAAATTACACA | |
| | ATTGACGCTGAGGCAGCTACGAAAAGAAAC | |
| | ATAGAAAATATGATAGGTACGATTCAGATT | |
| | CCACTAGGTGTCGCCGGTGCAATTATGGTG | |
| | AATGGCGAATATGCATCTGGAGAATTTATG | |
| | TTACCCCTGGCTACCACAGAAGGTGCATTA | |
| | GTCGCATCTGTTAATAGGGGATGCACGGTT | |
| | ATCACTGCTTCTGGGGGATCTAATGTGCGT | |
| | ATCTTCCAGGATCTGATGACCAGAGCCCCA | |
| | GTTTTCAAGTTGGAGAATGTGAATAAAGTT | |
| | AAAGAATTCGTTGATTGGGTAAAGAGAGAG | |
| | GAGACTTTCACCAATATGAAAGAGAAAGCC | |
| | GGAGAAACAACTAGATTTGGGGAGTTGTTG | |
| | TCCGTCGATCCCTTCATTACTGGTAACACG | |
| | GTTTTTCTTAGATTTGCTTACGATACTAAG | |
| | GACGCTATGGGGATGAACATGGTAACTATA | |
| | GCTACAGATGCGGTTTTAAATTTTATTTCC | |
| | GAGGATTTCGGCGTGTATCCGATCTCTTTA | |
| | AGTGGTAACATGTGTACGGATAAAAAACCA | |
| | GCGGCGATTAACAATATTTTAGGCAGGGGA | |
| | AAAACTGTTGCTGCTGATGTAACTATTCCT | |
| | AAAGAAATTGTCGAGAAAAAATTAAAAACC | |
| | ACACCAAAGATGATGGAAGAAGTCAACTAT | |
| | AGGAAGAATCTGTTGGGCTCTGCAAGGGCT | |
| | GGTGCCCTGGGCTTCAACGCTCACGCGGCT | |
| | AATATAATAGCCGCATTATATTTGGCTTGC | |
| | GGCCAAGATGCGGCACATGTTGTCGAAGGG | |
| | TCTAGTGCCATTACTACAATGGAAGTAAAT | |
| | GAAAATGGTGATTTGTACTGTTCGGTTACA | |
| | CTACCTAGCATACAAGTAGGTACAGTCGGT | |
| | GGAGGTACTGGTATCGCCACTCAAAGAGAT | |
| | TGCCTAAATTTGCTGGGTGTAGCTGGAGCT | |
| | GGTGAGGTACCTGGTCATAATTCAAAAAAG | |
| | CTAGCTGAAATTATTGCTGCCGCAGTCCTG | |
| | GCTGGAGAAATCTCCTTGATTGGTGCTCAA | |
| | GCAGCTGGCCACCTTGCTAAAGCACACGCC | |
| | GAATTGGGTAGATAA | |
| MvHMGR | ATGTTTTTAAAAGATAATGATCTTACAGAA | 28 |
| | GATGAAAAATTGTTGTTGCAAAAGGTTTTG | |
| | GATGGTGACATCGCTTTTAGGAAGATCGAG | |
| | GAATTTGCAGACCCGCTTACGGCAGTCAAG | |
| | ATTCGTCGTTTAGCTATACAGGAGTACGCC | |
| | AAATTGGAATTTGAACACATCCAAAATTTC | |
| | TCATTGGACGTTGAAACTGTAACTAAGAAA | |
| | AATATTGAAAACATGATCGGAGCCGTTCAA | |
| | ATACCATTAGGGGTTGCCGGGTTACTAAAG | |
| | GTTAATGGTGAGTACGCTGACGCAGAGTAC | |
| | TACATTCCATTAGCTACAACAGAAGGCGCC | |
| | CTGGTAGCTAGTGTAAATAGAGGCTGTTCA | |
| | GTAATTACTAAATCAGGAGGTGCTAATGTT | |
| | AGAGTATTCGAGGATGAAATGACTAGGGCT | |
| | CCGGTCTTTAAACTTGAAAGTTTAGATAGA | |
| | ACCAAAAAGTTCTATGAGTGGGTTAAAAGT | |
| | CCCGAGATCTTTGAACAAATGAAGACTGTT | |
| | GCGGAGAAGACGACAAGATTTGGTAAATTA | |
| | TTGTCTGTTAAGCCATTCGTGACCGGCACT | |
| | TATGTTTATCTTAGATTCTCGTACGATACC | |
| | AAGGACGCCATGGGCATGAATATGGTGACT | |
| | ATAGCAACGGATGCTGTTATGCATCTTATT | |
| | GAAGACGAATTTGGTGCCCACCCTATCACA | |
| | CTAAGTGGTAATATGTGCACCGATAAAAAA | |
| | CCAGCTTCAATCTCTACCATCTTAGGAAGA | |
| | GGTAAAACTGTAGTCGCTGAGGTTACTATC | |
| | CCCGAAGAAATTGTAAAGGAAACGTTGAAG | |
| | TGTACACCAGACGCTATGTTCGAAGTCAAT | |
| | TACAGTAAAAATCTACTAGGGTCTGCAAGA | |
| | GCCGGTGCGTTAGGTTTTAACGCTCACGCC | |
| | GCCAACGTTATAGCTGCAGTGTACTTAGCA | |
| | TGTGGTCAAGATGCAGCGCATGTAGTGGAA | |
| | GGTTCCACTGCTATAACCAGTATGGAATTA | |
| | ACTAAATATGGTGAGATTCACTGTTCAGTG | |
| | ACCTTACCCGCCTTGCCTGTTGGTACTGTG | |
| | GGAGGCGGCACGGGATTGGGTACCCAAAGG | |
| | GATTGTTTGAATATATTAGGCGTCGCCGGT | |
| | ACTGGGGATATACCAGGCATTAATTCAAGA | |
| | AAATTCGCCGAAATTGTTGCTAGTGCAGTT | |
| | TTAGCCGGCGAGATTTCGTTGATCGGTGCG | |
| | CAGGCAGCTGGACACCTGGCTCGTGCCCAC | |
| | GCCCAATTGGGTAGAGGAAAATTCTAA | |
| Pmev MvaA | ATGTCTTTAGACAGTAGGTTACCTGCTTTT | 29 |
| | AGAAATTTATCCCCTGCTGCCAGATTGGAT | |
| | CATATAGGTCAATTGCTTGGGCTATCACAC | |
| | GACGACGTTTCTCTTCTAGCTAATGCAGGA | |
| | GCCTTGCCGATGGACATAGCTAATGGTATG | |
| | ATCGAAAATGTGATTGGTACCTTTGAATTG | |
| | CCCTACGCAGTAGCTTCTAACTTTCAGATA | |
| | AACGGTAGGGACGTACTTGTACCATTGGTG | |
| | GTTGAAGAGCCCTCAATAGTCGCTGCTGCT | |
| | TCATACATGGCCAAGTTAGCCCGTGCCAAC | |
| | GGTGGTTTCACTACCAGCTCATCGGCTCCA | |
| | TTGATGCACGCACAAGTCCAGATAGTCGGT | |
| | ATCCAAGATCCACTGAATGCCAGACTGTCT | |
| | TTGTTGAGAAGAAAGGACGAAATCATAGAG | |
| | TTAGCAAATAGAAAGGATCAACTTTTAAAC | |
| | TCATTAGGAGGTGGCTGTCGTGATATCGAG | |
| | GTACATACTTTTGCCGACACTCCAAGAGGC | |
| | CCTATGTTGGTCGCACATTTAATAGTTGAT | |
| | GTACGTGATGCTATGGGCGCGAATACAGTG | |
| | AACACAATGGCCGAAGCGGTAGCTCCATTG | |
| | ATGGAAGCCATAACAGGAGGTCAAGTTCGT | |
| | CTACGTATTTTATCAAACCTAGCGGATCTG | |
| | AGATTGGCAAGAGCTCAAGTGAGGATCACC | |
| | CCGCAACAACTTGAAACTGCTGAATTTAGC | |
| | GGTGAGGCTGTGATTGAAGGTATCCTTGAC | |
| | GCTTATGCGTTTGCGGCAGTAGATCCCTAT | |
| | CGTGCTGCAACCCACAACAAGGGCATTATG | |
| | AATGGGATTGACCCGTTGATAGTGGCAACG | |
| | GGTAACGATTGGAGAGCAGTTGAAGCGGGT | |
| | GCTCATGCTTATGCATGTAGATCAGGACAT | |
| | TATGGTAGTCTAACTACCTGGGAGAAAGAT | |
| | AACAATGGACATTTGGTTGGTACACTAGAA | |
| | ATGCCTATGCCAGTTGGTTTAGTCGGAGGC | |
| | GCCACTAAAACACATCCTTTGGCACAACTA | |
| | TCTTTAAGAATATTGGGTGTAAAGACTGCC | |
| | CAAGCCTTGGCAGAAATTGCCGTCGCAGTC | |
| | GGTTTAGCGCAAAATCTTGGAGCTATGAGA | |
| | GCACTGGCCACCGAAGGCATCCAACGTGGC | |
| | CATATGGCATTGCATGCCAGAAATATTGCA | |
| | GTCGTTGCTGGGGCCAGGGGAGACGAAGTG | |
| | GATTGGGTTGCTAGACAATTAGTAGAATAC | |
| | CATGACGTTAGGGCTGATAGAGCGGTTGCC | |
| | CTTTTAAAGCAAAAGAGAGGACAATGA | |
| StMvaA | ATGACAAAGCTATCTTGGACGGGGTTTAGT | 30 |
| | AAGAAGACATTGCAAGAAAGAAAGGAGCAC | |
| | CTGAAAAATAATGCATTGTTATCCCAAGAA | |
| | AATCAAGATTTGCTTGACAATGACCAACAA | |
| | CTTACTCTAGAGACCGCAAATCAAATGGCA | |
| | GAAAATGTCATAGGAAGATTTACATTGCCT | |
| | TTTGCAATATGCCCAGACGTACTAGTGGAC | |
| | GGTGTAACATACCAAGTTCCGATGGTTACT | |
| | GAGGAACCTAGTGTTGTTGCAGCTGCCTCA | |
| | TATGCTAGCAAACTAATCAAAAGATCAGGT | |
| | GGTTTTACAACTAAGATACATGATAGACAG | |
| | ATGATTGGCCAAGTAGCCCTATTTGATGTT | |
| | CCCGATAAGGCCACGGCTGCCTCAAAAATA | |
| | CAGGCAGCGTCACAAAAGTTAATTGATATC | |
| | GCTAAGGAAGCGTACCCTTCCATAGTCAAA | |
| | AGAGGAGGCGGTCCTAGAAAATTGTGGACC | |
| | GAGACAAAAGGAGATTTTTTAATAGTGTAT | |
| | TTAGCAGTTGACACACAAGAAGCGATGGGT | |
| | GCAAATATGGTTAATACAATGATGGAAGCG | |
| | TTGGTTCCAGAATTGGAAAATTTGTCTGAG | |
| | GGACAATCATTAATGGCCATTTTGTCAAAT | |
| | TTGGCTACCGAATCCCTGGTAACAGCAACA | |
| | TGTAGGCTGAACACTAGATTCCTATCTAGA | |
| | AATAAGGCCGAAGCTCACAACTTCGCAAAA | |
| | AAAATGGAATTAGCTTCCCAGTTGGCACAA | |
| | GTGGATCCGTACAGAGCAGCAACCCATAAT | |
| | AAGGGTATTTTCAATGGAATAGACGCATTG | |
| | GTTATCGCTACAGGAAACGATTGGAGAGCT | |
| | GTCGAAGCTGGGTGCCATGCTTATGCTAGT | |
| | AAGGATGGTTCTTATAGAGGCCTTTCTACA | |
| | TGGACGTATAATCAGGAAACCAAAGAATTG | |
| | GTTGGTGAATTGACACTGCCTATGCCTATC | |
| | GCTACAAGAGGTGGTTCTATTGGTCTGAAT | |
| | CCTAGTGTTTCAATAGCACATGATCTTCTG | |
| | AATCACCCCGATGCTAGAACGCTAGCCGGT | |
| | ATCATAGTGTCACTAGGTCTGGTCCAGAAT | |
| | CTTGCTGCGCTGAAGGCTCTTACTAGCACT | |
| | GGCATACAGGCAGGCCATATGAAATTACAA | |
| | GCTAAGTCCTTGGCGTTGCTGGCTGGGGCG | |
| | AATCCGGAAGAAATGCCCCACGTGTTGTCT | |
| | GAATTACTTAAAGCAAAACATATGAACCAA | |
| | GAAACAGCTCAAGCAATCCTGGAAAAACTT | |
| | CGTAATCCGTAG | |
| ThMvaE | ATGAAGGACGTCGTAATAATTGATGCCCTA | 31 |
| | AGAACGCCGGTGGGAAAGTATCAAGGGTCT | |
| | TTATCACAGTTAAGTGCCGTTGAATTAGGC | |
| | TCTGCCGTGTCTAAGAAATTAATAAATAAC | |
| | AATAAGAAGGCGGCAGCCGCAATTAATCAG | |
| | GTGATATTTGGAAACGTTTTGCAAGCAGGA | |
| | TCTGGACAGAATCCAGCACGTCAAATCACG | |
| | TTAAATTCAGGACTATCTGAATCGGTATAT | |
| | GCCAGTACCATTAATGAAGTGTGTGGCTCT | |
| | GGTATGAAAGCGATTTCTCTTGCATCCCAA | |
| | GCTATTTTTTTAGATGAAGCTGAAGTTGTG | |
| | TTAGCTGGCGGAACAGAATCTATGTCGCAA | |
| | GCGCCTTATTTAAGCTATTACAATCAACAG | |
| | GAAGATACGTATAGTCAACCAAAGCCTGCT | |
| | ATGTTGTCTGATGGTTTGACTGATGTGTTT | |
| | AGTGGCCAACACATGGGGCTGACTGCCGAG | |
| | AATGTAGCCGAGAAGTTTAACATCACCAGA | |
| | AAAATGCAGGATGCGTTCGCCCTTAGATCT | |
| | CAAGAGAGAGCCGCAAATGCCCAGGAGAAA | |
| | GGCTATTTTAGCAATGAAATACTTCCGCTA | |
| | GATATAGCAGGTAAGAAGGTGGACAAAGAC | |
| | GAAGGTGTTCGTAAAGATACAAGCTTAGAA | |
| | AAGCTAGCAAAGTTAAAAACTGTCTTCAAA | |
| | AAAGAAGGCACGGTGACGGCTGGAAACGCT | |
| | TCTACAATTAACGATGGGGCGTCGGCTGTG | |
| | CTTTTGGCCTCAAAGAACTTTGCATTAGCC | |
| | AACGATTTATCCTACTTGGCAGTCTTGAAG | |
| | GATGTTGTGGAAGTAGGTGTCGACCCTAAA | |
| | GTTATGGGTATTTCACCCATAAAGGCCATT | |
| | AGGCAATTGCTAGAAAGAAATGCCTTGGCT | |
| | ATTGAGAATATTGATTTGTTTGAAATTAAT | |
| | GAAGCGTTCGCTTCGTCTAGTATAGCGGTA | |
| | GAGCAAGAACTAGAAATTCCTGAAGATAAG | |
| | GTTAACGTGTGTGGTTCGGGCATCTCTATC | |
| | GGCCACGCTATAGGCGCATCTGGCGCAAGG | |
| | ATTATTACAACAGCTTGTCATCAATTGGAA | |
| | AGAGTTGACGGCAGGTATGCAGTTGTTTCT | |
| | TTGTGTGTTGGTGGTGGTTTGGGATTAGCC | |
| | GCACTAATAGAAAGACCAAAAGCTAATAAG | |
| | AGCCACAAATTTTATCAATTGACTCGTAAG | |
| | GAGCGTTTGGATTTCCTGGTCAGTCATAAT | |
| | AAGATTACTTCAAAAACTGTTGACGAATTA | |
| | GAACGTACTGTCTTACCTGAATCAATCGCG | |
| | GGTAACTTAACAGAGAATCAGATGTCTGAA | |
| | ATTTCCTTACCCATGGGCTTGGTATCAAAT | |
| | ATGAGCGTAAATCAAAAGGATTACTTTGTA | |
| | CCTATGGCAACTGAGGAACCTTCAGTAGTT | |
| | GCTGCATGCAACAATGGTGTCCAAATGGCA | |
| | AAATCGAGTGGTGGTTTTACTGCAGTCATG | |
| | AAAAAGAAAGAAATCAGGGGACAAATTGTC | |
| | TTAATGAATGTCACCGACAAATCCACAGTT | |
| | ATAGAACAGATAGAAAAAAATGAAGCCGAG | |
| | ATTATATCCACTGCTGAGCAAAGTTACCCT | |
| | TCTATCGTGAAGAGGGGTGGCGGAGTCAAG | |
| | AGAGTCGTGGTCAGAGAGTTTGCTGAAGAC | |
| | CCTAATTTCTTAAGTGTTGATTTAATAGTT | |
| | GACACTCAGGATGCCATGGGTGCTAATATG | |
| | TTAAATACTATGTTAGAAGCTGTTGCAACT | |
| | CTGTTTAGGCAATGGTTTTCGGAAGAGATA | |
| | TTGTTCTCAATTTTATCAAACTACGCAACT | |
| | GATGCTTTGGTATCGGCTGAATGCTATATC | |
| | TCCTTTGCCAGTTTGGGAAAAGGTGATGCT | |
| | GAAAAGGGGGAGAAAATAGCAGAAAAGATA | |
| | GCTGCAGCTAGTAACTTCGCTCAAATAGAC | |
| | CCTTTCAGAGCCGCTACACACAATAAAGGT | |
| | ATTATGAATGGTATAGACGCTGTCGTTCTT | |
| | GCTACGGGGAACGATACTCGTTCTGTGAAT | |
| | AGTGCCGTACATGCTTATGCAGCTAAGAAC | |
| | GGTAAGTATCAAGGTTTATCCCAATGGGAG | |
| | ATTGTAGATAACCAGCTGAAAGGATCTATA | |
| | GAATTGCCTTTAGCGGTCGCCACTGCTGGT | |
| | GGGGCGACTAAAGTACTGCCAAAAGCGCAG | |
| | GCAGCGCTGCAGATCTTGGATGTAAATGAT | |
| | GCCAAAGAATTAGCAGAAGTCATCGCATCC | |
| | GTCGGTCTAGCACAAAACCTAGCCGCATTA | |
| | AAAGCCCTTGTTACTGAGGGTATACAAAAG | |
| | GGACATATGGCCTTGCAGGCAAGGACATTG | |
| | GCTCTTTCCGTAGGAGCAAAAGACTCCGAA | |
| | GTTCAGAAAGTTGCTAATAGATTGAAACGT | |
| | CAACAAATGAATGAGGAAAATGCTAGAAAA | |
| | ATTTTGCAGGAACTACGTAACCGTTAG | |
| ZmHMGR | ATGGAAGTTAGAGGTGGTGTGGGACAGGGC | 32 |
| | TCAGCTGCTAGACATCCTCCAGCTCCCGAG | |
| | CCTAGTCGTGCTGCTGCCAGGGTGCAGGCT | |
| | GGTGACGCTCTACCGTTACCCATAAGACAT | |
| | ACAAACTTGATTTTTAGTGCTCTGTTTGCA | |
| | GCGTCATTGGCCTATTTGATGCGTCGTTGG | |
| | AGAGAAAAAATAAGATCATCAACACCTCTT | |
| | CATGCCGTGGGATTGGCCGAAATGTTGGCA | |
| | ATTTTCGGACTGGTAGCTTCACTAATTTAT | |
| | CTTCTGAGCTTTTTCGGCATTGCCTTCGTA | |
| | CAATCTATTGTTTCCAGCGGAGATGATGAC | |
| | GAAGACTTCTTGGTCGGTTCTGGATCTTCA | |
| | GGATCGGCTGCAGCGCCCTCAAGACAACAT | |
| | GCTCAAGCACCAGCGCCATGTGAACTTCTG | |
| | GGTAGTCCAGCAGCTGCACCAGAGAAGATG | |
| | CCTGAGGATGATGAAGAAATTGTGGCCAGT | |
| | GTAGTGGCTGGAAAGGTCCCGAGTTACGCT | |
| | CTTGAGGCAAGATTGGGTGATTGCAGAAGA | |
| | GCTGCCGGGATTAGGCGTGAGGCATTAAGA | |
| | AGAATTACTGGTAGGGATATCGAAGGGTTA | |
| | CCCTTGGATGGCTTCGACTATGCATCCATC | |
| | CTAGGTCAGTGTTGTGAGTTGCCAGTTGGT | |
| | TACGTACAATTACCAGTCGGGGTAGCTGGA | |
| | CCTTTATTATTAGATGGACGTAGATTTTAC | |
| | CTGCCGATGGCGACCACGGAAGGTTGTTTG | |
| | GTTGCAAGCACAAATAGAGGATGTAAGGCT | |
| | ATTGCTGAATCTGGTGGTGCTACATCTGTA | |
| | GTGTTGAGGGATGCAATGACTCGTGCTCCA | |
| | GTCGCTCGTTTTCCGACCGCTCGTAGAGCC | |
| | GCTGAACTAAAGGCTTTTTTGGAAGATCCA | |
| | GCTAATTTCGATACATTAAGTGTGGTATTT | |
| | AATAGAAGCTCCCGTTTTGCAAGATTACAA | |
| | GGCGTCCAATGCGCAATGGCTGGTAGAAAT | |
| | TTGTACATGAGGTTTTCCTGTAGCACAGGT | |
| | GACGCGATGGGTATGAACATGGTTTCCAAG | |
| | GGAGTTCAAAATGTTTTAGATTTTCTTCAG | |
| | GATGACTTTCACGATATGGACGTAATTAGC | |
| | ATTTCCGGTAATTTTTGCTCAGACAAAAAA | |
| | CCTTCTGCTGTCAATTGGATTGAGGGTAGA | |
| | GGAAAGTCTGTTGTATGCGAAGCGGTCATT | |
| | GGTGAAGAAGTIGTAAAAAAAGTGTTAAAA | |
| | ACAGATGTTCAATCTTTAGTAGAATTAAAC | |
| | ACTATAAAAAATCTAGCTGGTTCTGCCGTT | |
| | GCCGGTGCTTTAGGGGGCTTCAACGCTCAC | |
| | GCTAGCAATATCGTTACTGCCATTTTTATT | |
| | GCGACCGGTCAAGATCCAGCCCAAAATGTC | |
| | GAGAGTAGTCATTGTATCACAATGCTGGAA | |
| | CCAGTCAACGCAGGTAGAGACCTTCATATA | |
| | TCAGTAACCATGCCTTCAATTGAGGTCGGC | |
| | ACTGTTGGTGGGGGTACTCAACTAGCCTCG | |
| | CAGTCTGCTTGCTTAGATCTATTGGGTGTT | |
| | AGAGGCGCGTCGAGGGACAGGCCCGGTTCG | |
| | AATGCAAGACTTCTAGCTACAGTTGTCGCG | |
| | GGGGGTGTGTTAGCCGGTGAGTTGTCTTTG | |
| | CTTTCAGCCCTAGCTGCCGGCCAATTGGTT | |
| | AAATCCCATATGAAATATAATAGGAGTTCC | |
| | AAAGATGTTTCTAGTACCACCGCAACTGAA | |
| | AAGACAAGACAACGTGAAGTGGATGTTTGA | |

Because the proteins disclosed herein may be of particular value when used in the context of transgenic expression in a microbial chassis, the nucleic acid sequence encoding any one of the proteins disclosed herein may be codon-optimized for a given expression system. For example, the nucleic acid sequence may be codon-optimized for expression in a yeast system, such as S. cerevisiae. Alternatively, the nucleic acid sequence may be codon-optimized for expression in a prokaryotic system, such as E. coli.

The nucleic acids disclosed herein that encode a HMGR enzyme can be incorporated into an expression vector or expression cassette. The nucleic acid can be transduced or transformed into a transgenic cell (e.g., yeast, E. coli, or another suitable microbe) such that the nucleic acid sequence encoding the HMGR enzyme is integrated into the genome of the host cell or transgenic cell. Alternatively, the nucleic acid sequence encoding the HMGR enzyme may be expressed without integration into the host genome (e.g., in the form of a plasmid). For those implementations in which genome integration is desired, any suitable methods of integration can be used, including but not limited to Cas-based systems (e.g., Cas9, Cas12, etc.), homologous recombination, gene gun, conjugation protocols, lambda red, etc. In some implementations, a nucleic acid encoding more than one of the HMGR enzymes disclosed herein (e.g., 2, 3, 4, 5, or more enzymes) may be integrated into the genome or otherwise expressed in a transgenic cell, as discussed in further detail below.

An expression cassette or vector for expressing the nucleic acid sequence encoding a HMGR enzyme disclosed herein may comprise a promoter and a terminator. Known promoters that can be used are well established in the art, and include but are not limited to GAL1, TEF2, TEF1, TDH3, ENO2, CCW12, EF-1a promoter, CMV immediate early, HSV thymidine kinase, early and late SV40, LTRs from retrovirus, and mouse metallothionein-I. In some implementations, an inducible or repressible promoter, such as GAL1, GAL2, GAL7, GAL10, CUP1, MET3, MET17, or MET25, may be used. Inducible promoters operably link the expression of a target gene (e.g., the nucleic acid sequence encoding a bakuchiol-producing protein) to a specific signal or a particular biotic or abiotic factor. Types of inducible promoters that may be utilized in the disclosed include, but are not limited to, chemically-inducible promoters (i.e., antibiotics, steroids, metals, etc.), light-inducible promoters, heat-inducible promoters, and hypoxia-inducible promoters. Transcription terminators that may be used are also known in the art, and include but are not limited to GAT2, Rho-dependent terminators, Rho-independent terminators, poly-A sequences, and the like.

For the purposes of the present disclosure, any of the foregoing proteins can be expressed in a host cell or transgenic cell and any of the foregoing nucleic acids may incorporated into a host cell or transgenic cell in order to produce isoprenoids according to the disclosed methods.

### IV. Host Cells and Transgenic Cells

Bioproduction of various isoprenoids can rely on a host cell that expresses a bakuchiol-producing protein as disclosed herein or a transgenic cell that expresses one or more of the HMGR enzymes disclosed herein. In one implementation, a host cell natively expresses a HMGR enzyme. In another implementation, a host cell does not natively express a HMGR enzyme. In one implementation, a transgenic cell does not natively express at least one of the HMGR enzymes disclosed herein.

For the purposes of this disclosure, expression of the HMGR enzymes disclosed herein can be heterologous, meaning the nucleic acid encoding one or more of the HMGR enzymes disclosed herein is derived from a species that is different from the species of the host cell or transgenic cell. Moreover, the disclosed host cells and transgenic cells may express one or more (e.g., 1, 2, 3, 4, 5, 6, 7, 8, or 10 or more) of the HMGR enzymes disclosed herein. Expression of combinations of the heterologous HMGR enzymes disclosed herein represents a beneficial improvement over current expression systems that rely on multiple gene copies of a single HMGR enzyme (which may be endogenous to the host cell) because this allows for increased flux to mevalonate while maintaining genomic stability. In contrast, prior systems that relied on multiple copies of the same gene may be intrinsically unstable due to the increased likelihood of recombination events. Accordingly, the cells disclosed herein that express one or more (e.g., 1, 2, 3, 4, 5, 6, 7, 8, 9, or 10 or more) of the heterologous HMGR enzymes disclosed herein can be used to boost production of all terpenes without compromising genetic stability.

The present disclosure provides an isolated host cell or a transgenic cell that expresses at least one (e.g., 1, 2, 3, 4, 5, 6, 7, 8, or 10 or more) of the HMGR enzyme(s) disclosed herein. In one aspect, the present disclosure provides a transgenic cell that comprises a transgene encoding at least one (e.g., 1, 2, 3, 4, 5, 6, 7, 8, or 10) of the HMGR enzyme(s) disclosed herein. In some implementations, the isolated host cell or transgenic cell may express at least one (e.g., 1, 2, 3, 4, 5, 6, 7, 8, or 10) of the HMGR enzyme(s) disclosed herein that has at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with any one of SEQ ID NOs: 1-16. In some implementations, the isolated host cell or transgenic cell may express one or more HMGR enzyme(s) that share at least 90% identity with one or more of SEQ ID NOs: 1-16. In some implementations, the isolated host cell or transgenic cell may express one or more HMGR enzyme(s) that share at least 95% identity with one or more of SEQ ID NOs: 1-16. In some implementations, the isolated host cell or transgenic cell may express one or more HMGR enzyme(s) that share at least 99% identity with one or more of SEQ ID NOs: 1-16. Thus, this disclosure contemplates and encompasses expression of proteins with varying degrees of sequence identity compared to SEQ ID NOs: 1-16, so long as the protein exhibits HMGR activity (i.e., the protein should be able to convert HMG-CoA to mevalonate).

In some implementations, an isolated host cell or transgenic cell of the present disclosure can express one or more of the HMGR enzymes disclosed herein (e.g., an enzyme comprising an amino acid sequence selected from any one of SEQ ID NOs: 1-16 or a variant thereof). In some implementations, an isolated host cell of transgenic cell of the present disclosure may comprise-either in its genome or as an extra-genomic sequence, such as a plasmid—a nucleic acid encoding one or more of the HMGR enzymes disclosed herein (e.g., a nucleic acid comprising a sequence of any one of SEQ ID NOs: 17-32 or codon-optimized variants thereof).

In some implementations, an isolated host cell or transgenic cell of the present disclosure can express a protein having at least about 65%—e.g., at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 1. In some implementations, an isolated host cell or transgenic cell of the present disclosure can express a protein having an amino acid sequence that comprises SEQ ID NO: 1. In some implementations, an isolated host cell or transgenic cell of the present disclosure can express a protein having an amino acid sequence that consists of SEQ ID NO: 1. In some implementations, an isolated host cell or transgenic cell of the present disclosure can express a protein consisting of 486 amino acids that have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 1. In some implementations, an isolated host cell or transgenic cell of the present disclosure can express a protein having more than 486 amino acids, but wherein about 486 of the total amino acids have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 1.

In some implementations, an isolated host cell or transgenic cell of the present disclosure can express a protein having at least about 65%—e.g., at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 2. In some implementations, an isolated host cell or transgenic cell of the present disclosure can express a protein having an amino acid sequence that comprises SEQ ID NO: 2. In some implementations, an isolated host cell or transgenic cell of the present disclosure can express a protein having an amino acid sequence that consists of SEQ ID NO: 2. In some implementations, an isolated host cell or transgenic cell of the present disclosure can express a protein consisting of 530 amino acids that have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 2. In some implementations, an isolated host cell or transgenic cell of the present disclosure can express a protein having more than 530 amino acids, but wherein about 530 of the total amino acids have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 2.

In some implementations, an isolated host cell or transgenic cell of the present disclosure can express a protein having at least about 65%—e.g., at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 3. In some implementations, an isolated host cell or transgenic cell of the present disclosure can express a protein having an amino acid sequence that comprises SEQ ID NO: 3. In some implementations, an isolated host cell or transgenic cell of the present disclosure can express a protein having an amino acid sequence that consists of SEQ ID NO: 3. In some implementations, an isolated host cell or transgenic cell of the present disclosure can express a protein consisting of 577 amino acids that have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 3. In some implementations, an isolated host cell or transgenic cell of the present disclosure can express a protein having more than 577 amino acids, but wherein about 577 of the total amino acids have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 3.

In some implementations, an isolated host cell or transgenic cell of the present disclosure can express a protein having at least about 65%—e.g., at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 4. In some implementations, an isolated host cell or transgenic cell of the present disclosure can express a protein having an amino acid sequence that comprises SEQ ID NO: 4. In some implementations, an isolated host cell or transgenic cell of the present disclosure can express a protein having an amino acid sequence that consists of SEQ ID NO: 4. In some implementations, an isolated host cell or transgenic cell of the present disclosure can express a protein consisting of 572 amino acids that have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 4. In some implementations, an isolated host cell or transgenic cell of the present disclosure can express a protein having more than 572 amino acids, but wherein about 572 of the total amino acids have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 4.

In some implementations, an isolated host cell or transgenic cell of the present disclosure can express a protein having at least about 65%—e.g., at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 5. In some implementations, an isolated host cell or transgenic cell of the present disclosure can express a protein having an amino acid sequence that comprises SEQ ID NO: 5. In some implementations, an isolated host cell or transgenic cell of the present disclosure can express a protein having an amino acid sequence that consists of SEQ ID NO: 5 In some implementations, an isolated host cell or transgenic cell of the present disclosure can express a protein consisting of 527 amino acids that have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 5. In some implementations, an isolated host cell or transgenic cell of the present disclosure can express a protein having more than 527 amino acids, but wherein about 527 of the total amino acids have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 5.

In some implementations, an isolated host cell or transgenic cell of the present disclosure can express a protein having at least about 65%—e.g., at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 6. In some implementations, an isolated host cell or transgenic cell of the present disclosure can express a protein having an amino acid sequence that comprises SEQ ID NO: 6. In some implementations, an isolated host cell or transgenic cell of the present disclosure can express a protein having an amino acid sequence that consists of SEQ ID NO: 6. In some implementations, an isolated host cell or transgenic cell of the present disclosure can express a protein consisting of 521 amino acids that have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 6. In some implementations, an isolated host cell or transgenic cell of the present disclosure can express a protein having more than 521 amino acids, but wherein about 521 of the total amino acids have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 6.

In some implementations, an isolated host cell or transgenic cell of the present disclosure can express a protein having at least about 65%—e.g., at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 7. In some implementations, an isolated host cell or transgenic cell of the present disclosure can express a protein having an amino acid sequence that comprises SEQ ID NO: 7. In some implementations, an isolated host cell or transgenic cell of the present disclosure can express a protein having an amino acid sequence that consists of SEQ ID NO: 7 In some implementations, an isolated host cell or transgenic cell of the present disclosure can express a protein consisting of 429 amino acids that have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 7. In some implementations, an isolated host cell or transgenic cell of the present disclosure can express a protein having more than 429 amino acids, but wherein about 429 of the total amino acids have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 7.

In some implementations, an isolated host cell or transgenic cell of the present disclosure can express a protein having at least about 65%—e.g., at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 8. In some implementations, an isolated host cell or transgenic cell of the present disclosure can express a protein having an amino acid sequence that comprises SEQ ID NO: 8. In some implementations, an isolated host cell or transgenic cell of the present disclosure can express a protein having an amino acid sequence that consists of SEQ ID NO: 8. In some implementations, an isolated host cell or transgenic cell of the present disclosure can express a protein consisting of 803 amino acids that have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 8. In some implementations, an isolated host cell or transgenic cell of the present disclosure can express a protein having more than 803 amino acids, but wherein about 803 of the total amino acids have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 8.

In some implementations, an isolated host cell or transgenic cell of the present disclosure can express a protein having at least about 65%—e.g., at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 9. In some implementations, an isolated host cell or transgenic cell of the present disclosure can express a protein having an amino acid sequence that comprises SEQ ID NO: 9. In some implementations, an isolated host cell or transgenic cell of the present disclosure can express a protein having an amino acid sequence that consists of SEQ ID NO: 9. In some implementations, an isolated host cell or transgenic cell of the present disclosure can express a protein consisting of 403 amino acids that have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 9. In some implementations, an isolated host cell or transgenic cell of the present disclosure can express a protein having more than 403 amino acids, but wherein about 403 of the total amino acids have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 9.

In some implementations, an isolated host cell or transgenic cell of the present disclosure can express a protein having at least about 65%—e.g., at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 10. In some implementations, an isolated host cell or transgenic cell of the present disclosure can express a protein having an amino acid sequence that comprises SEQ ID NO: 10. In some implementations, an isolated host cell or transgenic cell of the present disclosure can express a protein having an amino acid sequence that consists of SEQ ID NO: 10. In some implementations, an isolated host cell or transgenic cell of the present disclosure can express a protein consisting of 812 amino acids that have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 10. In some implementations, an isolated host cell or transgenic cell of the present disclosure can express a protein having more than 812 amino acids, but wherein about 812 of the total amino acids have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 10.

In some implementations, an isolated host cell or transgenic cell of the present disclosure can express a protein having at least about 65%—e.g., at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 11. In some implementations, an isolated host cell or transgenic cell of the present disclosure can express a protein having an amino acid sequence that comprises SEQ ID NO: 11. In some implementations, an isolated host cell or transgenic cell of the present disclosure can express a protein having an amino acid sequence that consists of SEQ ID NO: 11. In some implementations, an isolated host cell or transgenic cell of the present disclosure can express a protein consisting of 414 amino acids that have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 11. In some implementations, an isolated host cell or transgenic cell of the present disclosure can express a protein having more than 414 amino acids, but wherein about 414 of the total amino acids have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 11.

In some implementations, an isolated host cell or transgenic cell of the present disclosure can express a protein having at least about 65%—e.g., at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 12. In some implementations, an isolated host cell or transgenic cell of the present disclosure can express a protein having an amino acid sequence that comprises SEQ ID NO: 12. In some implementations, an isolated host cell or transgenic cell of the present disclosure can express a protein having an amino acid sequence that consists of SEQ ID NO: 12. In some implementations, an isolated host cell or transgenic cell of the present disclosure can express a protein consisting of 418 amino acids that have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 12. In some implementations, an isolated host cell or transgenic cell of the present disclosure can express a protein having more than 418 amino acids, but wherein about 418 of the total amino acids have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 12.

In some implementations, an isolated host cell or transgenic cell of the present disclosure can express a protein having at least about 65%—e.g., at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 13. In some implementations, an isolated host cell or transgenic cell of the present disclosure can express a protein having an amino acid sequence that comprises SEQ ID NO: 13. In some implementations, an isolated host cell or transgenic cell of the present disclosure can express a protein having an amino acid sequence that consists of SEQ ID NO: 13. In some implementations, an isolated host cell or transgenic cell of the present disclosure can express a protein consisting of 428 amino acids that have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 13. In some implementations, an isolated host cell or transgenic cell of the present disclosure can express a protein having more than 428 amino acids, but wherein about 428 of the total amino acids have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 13.

In some implementations, an isolated host cell or transgenic cell of the present disclosure can express a protein having at least about 65%—e.g., at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 14. In some implementations, an isolated host cell or transgenic cell of the present disclosure can express a protein having an amino acid sequence that comprises SEQ ID NO: 14. In some implementations, an isolated host cell or transgenic cell of the present disclosure can express a protein having an amino acid sequence that consists of SEQ ID NO: 14. In some implementations, an isolated host cell or transgenic cell of the present disclosure can express a protein consisting of 423 amino acids that have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 14. In some implementations, an isolated host cell or transgenic cell of the present disclosure can express a protein having more than 423 amino acids, but wherein about 423 of the total amino acids have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 14.

In some implementations, an isolated host cell or transgenic cell of the present disclosure can express a protein having at least about 65%—e.g., at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 15. In some implementations, an isolated host cell or transgenic cell of the present disclosure can express a protein having an amino acid sequence that comprises SEQ ID NO: 15. In some implementations, an isolated host cell or transgenic cell of the present disclosure can express a protein having an amino acid sequence that consists of SEQ ID NO: 15. In some implementations, an isolated host cell or transgenic cell of the present disclosure can express a protein consisting of 808 amino acids that have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 15. In some implementations, an isolated host cell or transgenic cell of the present disclosure can express a protein having more than 808 amino acids, but wherein about 808 of the total amino acids have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 15.

In some implementations, an isolated host cell or transgenic cell of the present disclosure can express a protein having at least about 65%—e.g., at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 16. In some implementations, an isolated host cell or transgenic cell of the present disclosure can express a protein having an amino acid sequence that comprises SEQ ID NO: 16. In some implementations, an isolated host cell or transgenic cell of the present disclosure can express a protein having an amino acid sequence that consists of SEQ ID NO: 16. In some implementations, an isolated host cell or transgenic cell of the present disclosure can express a protein consisting of 579 amino acids that have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 16. In some implementations, an isolated host cell or transgenic cell of the present disclosure can express a protein having more than 579 amino acids, but wherein about 579 of the total amino acids have at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with SEQ ID NO: 16.

In some implementations, an isolated host cell or transgenic cell can express one or more protein(s) (e.g., 1, 2, 3, 4, 5, 6, 7, 8, 9, or 10 or more) that have at least about 90% identity with SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, or SEQ ID NO: 16. In some implementations, an isolated host cell or transgenic cell can express one or more protein(s) (e.g., 1, 2, 3, 4, 5, 6, 7, 8, 9, or 10 or more) that have at least about 95% identity with SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, or SEQ ID NO: 16. In some implementations, an isolated host cell or transgenic cell can express one or more protein(s) (e.g., 1, 2, 3, 4, 5, 6, 7, 8, 9, or 10 or more) that have at least about 99% identity with SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, or SEQ ID NO: 16. Thus, this disclosure contemplates and encompasses host cells and transgenic cells that express heterologous proteins with varying degrees of sequence identity compared to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, or SEQ ID NO: 16, so long as the protein(s) exhibits HMGR activity, is able to produce mevalonate, or both.

As noted above, HMGR enzymes may be structurally similar and comprise conserved regions or domains. Thus, SEQ ID NOs: 1-16 and other HMGR enzymes within the scope of this disclosure may share a high degree of structural homology. Further, given the conversed regions and domains of HMGRs, the present disclosure contemplates HMGR enzyme variants in which the native inhibition domain has be removed or deleted (see, e.g., SEQ ID NOs: 1-6). Removal of the inhibitory domain (which is usually present only in animal-derived HMGR enzymes) may improve the activity level of the enzyme and, because the conversion of HMG-CoA to mevalonate is often rate limiting, improve the overall flux of isoprenoid production. Removal of the inhibitory domain may comprise a deletion of about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, about 300, about 305, about 310, about 315, about 320, about 325, about 330, about 335, about 340, about 345, about 350, about 355, about 360, about 365, about 370, about 375, about 380, about 385, about 390, about 395, or about 400 amino acids from the N-terminus of the protein. Thus, an isolated host cell or transgenic cell of the present disclosure may express one or more heterologous HMGR enzymes (e.g., 1, 2, 3, 4, 5, 6, 7, 8, 9, or 10 or more) comprising a deletion of an inhibitory domain at the N-terminus of the enzyme, as described above.

Examples of HMGR enzymes from which the N-terminal inhibitory domain has been removed include the truncated proteins represented by SEQ ID NOs: 1 (tAgHMGR_543), 2 (tDmHMGR_390), 3 (tEcHMGR_466), 4 (tFfHiMGR_610), 5 (tUnHMGR_487), and 6 (tHMGR_531). The removal of the N-terminal inhibitory domain in these proteins (i.e., SEQ ID NOs: 1-6) is based on sequence alignment to known structures/domains in HMGR enzymes. Nevertheless, in some implementations, further truncations may be possible while still maintaining HMGR activity. Thus, the present disclosure provides HMGR enzymes that are variants of any one of SEQ ID NOs: 1-6, in which up to 30, up to 25, up to 20, up to 15, up 14, up to 13, up to 12, up to 11, up to 10, up to 9, up to 8, up to 7, up to 6, up to 5, up to 4, up to 3, up to 2, or 1 amino acid(s) is/are removed from the N-terminus of any one of SEQ ID NOs: 1-6. Similarly, present disclosure provides HMGR enzymes that are variants of any one of SEQ ID NOs: 1-6, in which up to 30, up to 25, up to 20, up to 15, up 14, up to 13, up to 12, up to 11, up to 10, up to 9, up to 8, up to 7, up to 6, up to 5, up to 4, up to 3, up to 2, or 1 amino acid(s) is/are added to the N-terminus of any one of SEQ ID NOs: 1-6.

By the same token, the present disclosure likewise provides host cells or transgenic cells that express truncated variants of SEQ ID NOs: 7-16. In such variants, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, about 300, about 305, about 310, about 315, about 320, about 325, about 330, about 335, about 340, about 345, about 350, about 355, about 360, about 365, about 370, about 375, about 380, about 385, about 390, about 395, or about 400 amino acids may be deleted from the N-terminus of any one of SEQ ID NOs: 7-16. For the purposes of such variants, HMGR activity should be maintained (i.e., the enzyme should be able to convert HMG-CoA to mevalonate).

Various prokaryotic and eukaryotic expression systems are commonly used for bioproduction of terpenes and isoprenoids, though factors including the growth conditions, type of fermenter utilized, toxicity (if any) of the product, and other metabolic considerations of the microbe producing the product of interest may be employed to select a suitable system. Thus, the various prokaryotic and eukaryotic expression systems disclosed herein may be suitable as a host cell or transgenic cell that can be utilized, for example, in the methods of bioproduction of isoprenoids disclosed herein. In some implementations, a host cell or a transgenic cell suitable for expressing the HMGR enzymes disclosed herein may be a prokaryote. In some implementations, a host cell or a transgenic cell suitable for expressing the HMGR enzymes disclosed herein may be a eukaryote.

In some implementations, the isolated host cell or transgenic cell is a prokaryote. Model prokaryotic systems that may be utilized as a transgenic cell include but are not limited to Escherichia coli (E. coli), an Acinetobacter species, a Pseudomonas species, a Streptomyces species, and a Mycobacterium species. Additional suitable prokaryotic expression systems include, but are not limited to, Klebsiella, Lactococcus, Mannheimia, Corynebacterium, Vibrio, and Bacillis.

In some implementations, the isolated host cell or transgenic cell is a eukaryote. Model eukaryotic systems that may be utilized as a transgenic cell include but are not limited to Saccharomyces cerevisiae (S. cerevisiae) or other yeast species; a filamentous fungi, optionally selected from an Aspergillus species and a Trichoderma species; an algae, optionally selected from Botryococcus braunii, Chlorella sp., Crypthecodinium cohnii, Cylindrotheca sp., Nitzschia sp., Phaeodactylum tricornutum, Schizochytrium sp., and Tetraselmis suecia; and an amoeba, which is optionally Dictyostelium discoideum. Additional suitable eukaryotic expression systems include, but are not limited to, Pichia pastoris, Yarrowia lipolytica, Kluyveromyces marxianus, Rhodosporidium toruloides. Aspergillus (oryzae, nidulans, niger), Trichoderma reesei, and Penicillium chrysogenum.

As noted above, in implementations involving a transgenic cell (e.g., S. cerevisiae or E. coli), the transgenic cell may comprise a transgene or transgenes encoding one or more of the HMGR enzymes disclosed herein (e.g., 1, 2, 3, 4, 5, 6, 7, 8, 9, 10 or more of the HMGR enzymes), and the transgene(s) can be integrated into the transgenic cell's genome. The transgene may be integrated within an expression cassette that appropriately drives expression of the HMGR enzyme(s). For those implementations in which genome integration of the transgene is desired, known suitable methods of integration can be used, including but not limited to Cas-based systems (e.g., Cas9, Cas12, etc.), homologous recombination, gene gun, conjugation protocols, lambda red, etc. Alternatively, in some implementations, the transgene may not be integrated into the genome, and instead the HMGR enzyme(s) may be expressed from, for example, a plasmid (e.g., an episomal plasmid) or similar vector.

An expression cassette or vector for expressing the transgene(s) may comprise one or more promoter(s) and terminator(s), depending on whether a HMGR enzyme disclosed herein is expressed alone or with an additional heterologous HMGR enzyme or additional heterologous enzymes. Suitable promoters that can be used may include but are not limited to GAL1, TEF2, TEF1, TDH3, ENO2, CCW12, EF-1a promoter, CMV immediate early, HSV thymidine kinase, early and late SV40, LTRs from retrovirus, and mouse metallothionein-I. In some implementations, the promoter is GAL1. In some implementations, an inducible or repressible promoter, such as GAL1, GAL2, GAL7, GAL10, CUP1, MET3, MET17, or MET25, may be used. Inducible promoters operably link the expression of a target gene (e.g., the nucleic acid sequence encoding a bakuchiol-producing protein) to a specific signal or a particular biotic or abiotic factor. Types of inducible promoters that may be utilized in the disclosed include, but are not limited to, chemically-inducible promoters (i.e., antibiotics, steroids, metals, etc.), light-inducible promoters, heat-inducible promoters, and hypoxia-inducible promoters. Transcription terminators that may be used are also known in the art, and include but are not limited to GAT2, Rho-dependent terminators, Rho-independent terminators, poly-A sequences, and the like. In some implementations, the terminator is GAT2.

### V. Methods of Bioproduction and Batches Produced Therefrom

The identification, isolation, and characterization of the HMGR enzymes disclosed herein allows methods of bioproduction of isoprenoids that are more efficient and produce higher yields than previous bioproduction methods by overcoming the rate-limiting step of mevalonate production. Thus, the present disclosure provides methods of producing isoprenoids, comprising culturing an isolated host cell or a transgenic cell disclosed herein in a culture medium. For the purposes of the disclosed methods, it is understood that the precise contents of the culture medium may vary depending on what isoprenoid product is desired and what type of host cell or transgenic cell is expressing one or more of the HMGR enzymes disclosed herein.

In some implementations, the methods comprise culturing a host cell or transgenic cell (e.g., S. cerevisiae or E. coli) comprising a transgene that encodes at least one HMGR enzyme that has at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with any one of SEQ ID NOs: 1-16.

In some implementations, the methods comprise culturing a host cell or transgenic cell (e.g., S. cerevisiae or E. coli) comprising one or more transgene(s) that encodes at least two HMGR enzymes that has at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with any two of SEQ ID NOs: 1-16.

In some implementations, the methods comprise culturing a host cell or transgenic cell (e.g., S. cerevisiae or E. coli) comprising one or more transgene(s) that encodes at least three HMGR enzymes that has at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with any three of SEQ ID NOs: 1-16.

In some implementations, the methods comprise culturing a host cell or transgenic cell (e.g., S. cerevisiae or E. coli) comprising one or more transgene(s) that encodes at least four HMGR enzymes that has at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with any four of SEQ ID NOs: 1-16.

In some implementations, the methods comprise culturing a host cell or transgenic cell (e.g., S. cerevisiae or E. coli) comprising one or more transgene(s) that encodes at least five HMGR enzymes that has at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with any five of SEQ ID NOs: 1-16.

In some implementations, the methods comprise culturing a host cell or transgenic cell (e.g., S. cerevisiae or E. coli) comprising one or more transgene(s) that encodes at least six HMGR enzymes that has at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with any six of SEQ ID NOs: 1-16.

In some implementations, the methods comprise culturing a host cell or transgenic cell (e.g., S. cerevisiae or E. coli) comprising one or more transgene(s) that encodes at least seven HMGR enzymes that has at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with any seven of SEQ ID NOs: 1-16.

In some implementations, the methods comprise culturing a host cell or transgenic cell (e.g., S. cerevisiae or E. coli) comprising one or more transgene(s) that encodes at least eight HMGR enzymes that has at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with any eight of SEQ ID NOs: 1-16.

In some implementations, the methods comprise culturing a host cell or transgenic cell (e.g., S. cerevisiae or E. coli) comprising one or more transgene(s) that encodes at least nine HMGR enzymes that has at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with any nine of SEQ ID NOs: 1-16.

In some implementations, the methods comprise culturing a host cell or transgenic cell (e.g., S. cerevisiae or E. coli) comprising one or more transgene(s) that encodes at least ten HMGR enzymes that has at least about 65%—e.g., at least about 70%, at least about 75% at least about 80%, at least about 85%, at least about 90%, at least about 91%, at least about 92%, at least about 93%, at least about 94%, at least about 95%, at least about 96%, at least about 97%, at least about 98%, at least about 99%, or 100%, or any values in between any of the two aforementioned values, identity with any ten of SEQ ID NOs: 1-16.

In some implementations, the HMGR enzyme(s) expressed by a host cell or transgenic cell may be share at least 90% identity with any 1, 2, 3, 4, 5, 6, 7, 8, 9, or 10 of SEQ ID NOs: 1-16. In some implementations, the HMGR enzyme(s) expressed by a host cell or transgenic cell may be share at least 91% identity with any 1, 2, 3, 4, 5, 6, 7, 8, 9, or 10 of SEQ ID NOs: 1-16. In some implementations, the HMGR enzyme(s) expressed by a host cell or transgenic cell may be share at least 92% identity with any 1, 2, 3, 4, 5, 6, 7, 8, 9, or 10 of SEQ ID NOs: 1-16. In some implementations, the HMGR enzyme(s) expressed by a host cell or transgenic cell may be share at least 93% identity with any 1, 2, 3, 4, 5, 6, 7, 8, 9, or 10 of SEQ ID NOs: 1-16. In some implementations, the HMGR enzyme(s) expressed by a host cell or transgenic cell may be share at least 94% identity with any 1, 2, 3, 4, 5, 6, 7, 8, 9, or 10 of SEQ ID NOs: 1-16. In some implementations, the HMGR enzyme(s) expressed by a host cell or transgenic cell may be share at least 95% identity with any 1, 2, 3, 4, 5, 6, 7, 8, 9, or 10 of SEQ ID NOs: 1-16. In some implementations, the HMGR enzyme(s) expressed by a host cell or transgenic cell may be share at least 96% identity with any 1, 2, 3, 4, 5, 6, 7, 8, 9, or 10 of SEQ ID NOs: 1-16. In some implementations, the HMGR enzyme(s) expressed by a host cell or transgenic cell may be share at least 97% identity with any 1, 2, 3, 4, 5, 6, 7, 8, 9, or 10 of SEQ ID NOs: 1-16. In some implementations, the HMGR enzyme(s) expressed by a host cell or transgenic cell may be share at least 98% identity with any 1, 2, 3, 4, 5, 6, 7, 8, 9, or 10 of SEQ ID NOs: 1-16. In some implementations, the HMGR enzyme(s) expressed by a host cell or transgenic cell may be share at least 99% identity with any 1, 2, 3, 4, 5, 6, 7, 8, 9, or 10 of SEQ ID NOs: 1-16. In some implementations, the HMGR enzyme(s) expressed by a host cell or transgenic cell may independently comprise amino acid sequences comprising or consisting of 1, 2, 3, 4, 5, 6, 7, 8, 9, or 10 of SEQ ID NOs: 1-16. Thus, the enzymes may possess varying degrees of sequence identity compared to SEQ ID NOs: 1-16, so long as HMGR activity is maintained (i.e., the enzyme should be able to convert HMG-CoA to mevalonate).

Various prokaryotic and eukaryotic expression systems can be utilized for the methods disclosed herein. In some implementations, the transgenic cell used in the methods may be a prokaryote, including but are not limited to Escherichia coli (E. coli), an Acinetobacter species, a Pseudomonas species, a Streptomyces species, and a Mycobacterium species. Additionally suitable prokaryotic expression systems include, but are not limited to, Klebsiella, Lactococcus, Mannheimia, Corynebacterium, Vibrio, and Bacillis. In in some implementations, the transgenic cell used in the methods may be a eukaryote, including but are not limited to Saccharomyces cerevisiae (S. cerevisiae) or other yeast species; a filamentous fungi, optionally selected from an Aspergillus species and a Trichoderma species; an algae, optionally selected from Botryococcus braunii, Chlorella sp., Crypthecodinium cohnii, Cylindrotheca sp., Nitzschia sp., Phaeodactylum tricornutum, Schizochytrium sp., and Tetraselmis suecia; and an amoeba, which is optionally Dictyostelium discoideum. Additional suitable eukaryotic expression systems include, but are not limited to, Pichia pastoris, Yarrowia lipolytica, Kluyveromyces marxianus, Rhodosporidium toruloides. Aspergillus (oryzae, nidulans, niger), Trichoderma reesei, and Penicillium chrysogenum. 101.301 The methods disclosed herein can be carried out in a bioproduction reactor, fermentation tank, culture flask, or other suitable containers for bioproduction. Various different culture mediums can be selected based on the particular transgenic species used and the growth conditions, among other things. In some implementations, minimal culture medium may be supplemented as needed to optimize growth and production of a given transgenic cell type.

The methods disclosed herein increase flux of mevalonate, which can improve efficiency and yield for any terpene or isoprenoid, as mevalonate is an integral in all terpene and isoprenoid biosynthetic pathways. These methods of bioproduction may be further optimized and developed to increase yield, and combining the HMGR enzymes disclosed herein in various ways may also vary the ultimate isoprenoid yield. As noted above, the overall increase in yield is believed to result from increase flux to mevalonate.

Accordingly, the present disclosure provides various methods of producing, improving production of, or increasing production of an isoprenoid, terpene, or terpenoid. In some implementations, the isoprenoid may be a sesquiterpene, a monoterpene, a diterpene, or a meroterpene. In specific implementations the isoprenoid may be a sesquiterpene. In specific implementations the isoprenoid may be a monoterpene. In specific implementations the isoprenoid may be a diterpene. In specific implementations the isoprenoid may be a meroterpene. In some implementations, the isoprenoid may be selected from bakuchiol, farnesene, farnesol, geosmin, geraniol, terpineol, limonene, myrcene, linalool, hinokitiol, pinene, cafestol, kahweol, cembrene, taxadiene, α-bisabolol, α-guaiene, bergamontene, and valencene. However, it should be noted that the isoprenoid, terpene, or terpenoid is not particularly limited, as mevalonate is a precursor involved in all isoprenoid compounds.

In particular implementations, the present disclosure provides methods of producing bakuchiol, comprising culturing a transgenic cell or host cell, as disclosed herein. As discussed herein, the transgenic cell may be a yeast cell, which can heterologously express at least one (e.g., 1, 2, 3, 4, 5, 6, 7, 8, 9, or 10 or more) of the HMGR enzymes disclosed herein or which comprises at least one nucleic acid encoding one or more of the HMGR enzymes disclosed herein. In such implementations bakuchiol production may be increased relative to a yeast cell that does not heterologously express at least one (e.g., 1, 2, 3, 4, 5, 6, 7, 8, 9, or 10 or more) of the HMGR enzymes disclosed herein.

In particular implementations, the present disclosure provides methods of producing farnesene, comprising culturing a transgenic cell or host cell, as disclosed herein. As discussed herein, the transgenic cell may be a yeast cell, which can heterologously express at least one (e.g., 1, 2, 3, 4, 5, 6, 7, 8, 9, or 10 or more) of the HMGR enzymes disclosed herein or which comprises at least one nucleic acid encoding one or more of the HMGR enzymes disclosed herein. In such implementations farnesene production may be increased relative to a yeast cell that does not heterologously express at least one (e.g., 1, 2, 3, 4, 5, 6, 7, 8, 9, or 10 or more) of the HMGR enzymes disclosed herein.

In particular implementations, the present disclosure provides methods of producing farnesol, comprising culturing a transgenic cell or host cell, as disclosed herein. As discussed herein, the transgenic cell may be a yeast cell, which can heterologously express at least one (e.g., 1, 2, 3, 4, 5, 6, 7, 8, 9, or 10 or more) of the HMGR enzymes disclosed herein or which comprises at least one nucleic acid encoding one or more of the HMGR enzymes disclosed herein. In such implementations farnesol production may be increased relative to a yeast cell that does not heterologously express at least one (e.g., 1, 2, 3, 4, 5, 6, 7, 8, 9, or 10 or more) of the HMGR enzymes disclosed herein.

In particular implementations, the present disclosure provides methods of producing geosmin, comprising culturing a transgenic cell or host cell, as disclosed herein. As discussed herein, the transgenic cell may be a yeast cell, which can heterologously express at least one (e.g., 1, 2, 3, 4, 5, 6, 7, 8, 9, or 10 or more) of the HMGR enzymes disclosed herein or which comprises at least one nucleic acid encoding one or more of the HMGR enzymes disclosed herein. In such implementations geosmin production may be increased relative to a yeast cell that does not heterologously express at least one (e.g., 1, 2, 3, 4, 5, 6, 7, 8, 9, or 10 or more) of the HMGR enzymes disclosed herein.

In particular implementations, the present disclosure provides methods of producing geraniol, comprising culturing a transgenic cell or host cell, as disclosed herein. As discussed herein, the transgenic cell may be a yeast cell, which can heterologously express at least one (e.g., 1, 2, 3, 4, 5, 6, 7, 8, 9, or 10 or more) of the HMGR enzymes disclosed herein or which comprises at least one nucleic acid encoding one or more of the HMGR enzymes disclosed herein. In such implementations geraniol production may be increased relative to a yeast cell that does not heterologously express at least one (e.g., 1, 2, 3, 4, 5, 6, 7, 8, 9, or 10 or more) of the HMGR enzymes disclosed herein.

In particular implementations, the present disclosure provides methods of producing terpineol, comprising culturing a transgenic cell or host cell, as disclosed herein. As discussed herein, the transgenic cell may be a yeast cell, which can heterologously express at least one (e.g., 1, 2, 3, 4, 5, 6, 7, 8, 9, or 10 or more) of the HMGR enzymes disclosed herein or which comprises at least one nucleic acid encoding one or more of the HMGR enzymes disclosed herein. In such implementations terpineol production may be increased relative to a yeast cell that does not heterologously express at least one (e.g., 1, 2, 3, 4, 5, 6, 7, 8, 9, or 10 or more) of the HMGR enzymes disclosed herein.

In particular implementations, the present disclosure provides methods of producing limonene, comprising culturing a transgenic cell or host cell, as disclosed herein. As discussed herein, the transgenic cell may be a yeast cell, which can heterologously express at least one (e.g., 1, 2, 3, 4, 5, 6, 7, 8, 9, or 10 or more) of the HMGR enzymes disclosed herein or which comprises at least one nucleic acid encoding one or more of the HMGR enzymes disclosed herein. In such implementations limonene production may be increased relative to a yeast cell that does not heterologously express at least one (e.g., 1, 2, 3, 4, 5, 6, 7, 8, 9, or 10 or more) of the HMGR enzymes disclosed herein.

In particular implementations, the present disclosure provides methods of producing myrcene, comprising culturing a transgenic cell or host cell, as disclosed herein. As discussed herein, the transgenic cell may be a yeast cell, which can heterologously express at least one (e.g., 1, 2, 3, 4, 5, 6, 7, 8, 9, or 10 or more) of the HMGR enzymes disclosed herein or which comprises at least one nucleic acid encoding one or more of the HMGR enzymes disclosed herein. In such implementations myrcene production may be increased relative to a yeast cell that does not heterologously express at least one (e.g., 1, 2, 3, 4, 5, 6, 7, 8, 9, or 10 or more) of the HMGR enzymes disclosed herein.

In particular implementations, the present disclosure provides methods of producing linalool, comprising culturing a transgenic cell or host cell, as disclosed herein. As discussed herein, the transgenic cell may be a yeast cell, which can heterologously express at least one (e.g., 1, 2, 3, 4, 5, 6, 7, 8, 9, or 10 or more) of the HMGR enzymes disclosed herein or which comprises at least one nucleic acid encoding one or more of the HMGR enzymes disclosed herein. In such implementations linalool production may be increased relative to a yeast cell that does not heterologously express at least one (e.g., 1, 2, 3, 4, 5, 6, 7, 8, 9, or 10 or more) of the HMGR enzymes disclosed herein.

In particular implementations, the present disclosure provides methods of producing hinokitiol, comprising culturing a transgenic cell or host cell, as disclosed herein. As discussed herein, the transgenic cell may be a yeast cell, which can heterologously express at least one (e.g., 1, 2, 3, 4, 5, 6, 7, 8, 9, or 10 or more) of the HMGR enzymes disclosed herein or which comprises at least one nucleic acid encoding one or more of the HMGR enzymes disclosed herein. In such implementations hinokitiol production may be increased relative to a yeast cell that does not heterologously express at least one (e.g., 1, 2, 3, 4, 5, 6, 7, 8, 9, or 10 or more) of the HMGR enzymes disclosed herein.

In particular implementations, the present disclosure provides methods of producing pinene, comprising culturing a transgenic cell or host cell, as disclosed herein. As discussed herein, the transgenic cell may be a yeast cell, which can heterologously express at least one (e.g., 1, 2, 3, 4, 5, 6, 7, 8, 9, or 10 or more) of the HMGR enzymes disclosed herein or which comprises at least one nucleic acid encoding one or more of the HMGR enzymes disclosed herein. In such implementations pinene production may be increased relative to a yeast cell that does not heterologously express at least one (e.g., 1, 2, 3, 4, 5, 6, 7, 8, 9, or 10 or more) of the HMGR enzymes disclosed herein.

In particular implementations, the present disclosure provides methods of producing cafestol, comprising culturing a transgenic cell or host cell, as disclosed herein. As discussed herein, the transgenic cell may be a yeast cell, which can heterologously express at least one (e.g., 1, 2, 3, 4, 5, 6, 7, 8, 9, or 10 or more) of the HMGR enzymes disclosed herein or which comprises at least one nucleic acid encoding one or more of the HMGR enzymes disclosed herein. In such implementations cafestol production may be increased relative to a yeast cell that does not heterologously express at least one (e.g., 1, 2, 3, 4, 5, 6, 7, 8, 9, or 10 or more) of the HMGR enzymes disclosed herein.

In particular implementations, the present disclosure provides methods of producing kahweol, comprising culturing a transgenic cell or host cell, as disclosed herein. As discussed herein, the transgenic cell may be a yeast cell, which can heterologously express at least one (e.g., 1, 2, 3, 4, 5, 6, 7, 8, 9, or 10 or more) of the HMGR enzymes disclosed herein or which comprises at least one nucleic acid encoding one or more of the HMGR enzymes disclosed herein. In such implementations kahweol production may be increased relative to a yeast cell that does not heterologously express at least one (e.g., 1, 2, 3, 4, 5, 6, 7, 8, 9, or 10 or more) of the HMGR enzymes disclosed herein.

In particular implementations, the present disclosure provides methods of producing cembrene, comprising culturing a transgenic cell or host cell, as disclosed herein. As discussed herein, the transgenic cell may be a yeast cell, which can heterologously express at least one (e.g., 1, 2, 3, 4, 5, 6, 7, 8, 9, or 10 or more) of the HMGR enzymes disclosed herein or which comprises at least one nucleic acid encoding one or more of the HMGR enzymes disclosed herein. In such implementations cembrene production may be increased relative to a yeast cell that does not heterologously express at least one (e.g., 1, 2, 3, 4, 5, 6, 7, 8, 9, or 10 or more) of the HMGR enzymes disclosed herein.

In particular implementations, the present disclosure provides methods of producing taxadiene, comprising culturing a transgenic cell or host cell, as disclosed herein. As discussed herein, the transgenic cell may be a yeast cell, which can heterologously express at least one (e.g., 1, 2, 3, 4, 5, 6, 7, 8, 9, or 10 or more) of the HMGR enzymes disclosed herein or which comprises at least one nucleic acid encoding one or more of the HMGR enzymes disclosed herein. In such implementations taxadiene production may be increased relative to a yeast cell that does not heterologously express at least one (e.g., 1, 2, 3, 4, 5, 6, 7, 8, 9, or 10 or more) of the HMGR enzymes disclosed herein.

In particular implementations, the present disclosure provides methods of producing α-bisabolol, comprising culturing a transgenic cell or host cell, as disclosed herein. As discussed herein, the transgenic cell may be a yeast cell, which can heterologously express at least one (e.g., 1, 2, 3, 4, 5, 6, 7, 8, 9, or 10 or more) of the HMGR enzymes disclosed herein or which comprises at least one nucleic acid encoding one or more of the HMGR enzymes disclosed herein. In such implementations α-bisabolol production may be increased relative to a yeast cell that does not heterologously express at least one (e.g., 1, 2, 3, 4, 5, 6, 7, 8, 9, or 10 or more) of the HMGR enzymes disclosed herein.

In particular implementations, the present disclosure provides methods of producing α-guaiene, comprising culturing a transgenic cell or host cell, as disclosed herein. As discussed herein, the transgenic cell may be a yeast cell, which can heterologously express at least one (e.g., 1, 2, 3, 4, 5, 6, 7, 8, 9, or 10 or more) of the HMGR enzymes disclosed herein or which comprises at least one nucleic acid encoding one or more of the HMGR enzymes disclosed herein. In such implementations α-guaiene production may be increased relative to a yeast cell that does not heterologously express at least one (e.g., 1, 2, 3, 4, 5, 6, 7, 8, 9, or 10 or more) of the HMGR enzymes disclosed herein.

In particular implementations, the present disclosure provides methods of producing bergamontene, comprising culturing a transgenic cell or host cell, as disclosed herein. As discussed herein, the transgenic cell may be a yeast cell, which can heterologously express at least one (e.g., 1, 2, 3, 4, 5, 6, 7, 8, 9, or 10 or more) of the HMGR enzymes disclosed herein or which comprises at least one nucleic acid encoding one or more of the HMGR enzymes disclosed herein. In such implementations bergamontene production may be increased relative to a yeast cell that does not heterologously express at least one (e.g., 1, 2, 3, 4, 5, 6, 7, 8, 9, or 10 or more) of the HMGR enzymes disclosed herein.

In particular implementations, the present disclosure provides methods of producing valencene, comprising culturing a transgenic cell or host cell, as disclosed herein. As discussed herein, the transgenic cell may be a yeast cell, which can heterologously express at least one (e.g., 1, 2, 3, 4, 5, 6, 7, 8, 9, or 10 or more) of the HMGR enzymes disclosed herein or which comprises at least one nucleic acid encoding one or more of the HMGR enzymes disclosed herein. In such implementations valencene production may be increased relative to a yeast cell that does not heterologously express at least one (e.g., 1, 2, 3, 4, 5, 6, 7, 8, 9, or 10 or more) of the HMGR enzymes disclosed herein.

In some implementations of the methods disclosed herein, heterologous expression of one or more of the HMGR enzymes disclosed herein in a transgenic or host cell may increase mevalonate production compared to the endogenous HMGR enzyme of the transgenic cell or host cell by at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2.0-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, at least 3.0-fold, at least 3.1-fold, at least 3.2-fold, at least 3.3-fold, at least 3.4-fold, at least 3.5-fold, at least 3.6-fold, at least 3.7-fold, at least 3.8-fold, at least 3.9-fold, at least 4.0-fold, at least 4.1-fold, at least 4.2-fold, at least 4.3-fold, at least 4.4-fold, at least 4.5-fold, at least 4.6-fold, at least 4.7-fold, at least 4.8-fold, at least 4.9-fold, at least 5.0-fold, at least 5.1-fold, at least 5.2-fold, at least 5.3-fold, at least 5.4-fold, at least 5.5-fold, at least 5.6-fold, at least 5.7-fold, at least 5.8-fold, at least 5.9-fold, at least 6.0-fold, at least 6.1-fold, at least 6.2-fold, at least 6.3-fold, at least 6.4-fold, at least 6.5-fold, at least 6.6-fold, at least 6.7-fold, at least 6.8-fold, at least 6.9-fold, at least 7.0-fold, at least 7.1-fold, at least 7.2-fold, at least 7.3-fold, at least 7.4-fold, at least 7.5-fold, at least 7.6-fold, at least 7.7-fold, at least 7.8-fold, at least 7.9-fold, at least 8.0-fold, at least 8.1-fold, at least 8.2-fold, at least 8.3-fold, at least 8.4-fold, at least 8.5-fold, at least 8.6-fold, at least 8.7-fold, at least 8.8-fold, at least 8.9-fold, at least 9.0-fold, at least 9.1-fold, at least 9.2-fold, at least 9.3-fold, at least 9.4-fold, at least 9.5-fold, at least 9.6-fold, at least 9.7-fold, at least 9.8-fold, at least 9.9-fold, at least 10.0-fold. at least 10.1-fold, at least 10.2-fold, at least 10.3-fold, at least 10.4-fold, at least 10.5-fold, at least 10.6-fold, at least 10.7-fold, at least 10.8-fold, at least 10.9-fold, at least 11.0-fold, at least 11.1-fold, at least 11.2-fold, at least 11.3-fold, at least 11.4-fold, at least 11.5-fold, at least 11.6-fold, at least 11.7-fold, at least 11.8-fold, at least 11.9-fold, at least 12.0-fold, at least 12.1-fold, at least 12.2-fold, at least 12.3-fold, at least 12.4-fold, at least 12.5-fold, at least 12.6-fold, at least 12.7-fold, at least 12.8-fold, at least 12.9-fold, at least 13.0-fold, at least 13.1-fold, at least 13.2-fold, at least 13.3-fold, at least 13.4-fold, at least 13.5-fold, at least 13.6-fold, at least 13.7-fold, at least 13.8-fold, at least 13.9-fold, at least 14.0-fold, at least 14.1-fold, at least 14.2-fold, at least 14.3-fold, at least 14.4-fold, at least 14.5-fold, at least 14.6-fold, at least 14.7-fold, at least 14.8-fold, at least 14.9-fold, or at least 15.0-fold.

In some implementations of the methods disclosed herein, heterologous expression of one or more of the HMGR enzymes disclosed herein in a transgenic or host cell may increase mevalonate production compared to the endogenous HMGR enzyme of the transgenic cell or host cell by about 1.1-fold, about 1.2-fold, about 1.3-fold, about 1.4-fold, about 1.5-fold, about 1.6-fold, about 1.7-fold, about 1.8-fold, about 1.9-fold, about 2.0-fold, about 2.1-fold, about 2.2-fold, about 2.3-fold, about 2.4-fold, about 2.5-fold, about 2.6-fold, about 2.7-fold, about 2.8-fold, about 29-fold, about 3.0-fold, about 3.1-fold, about 3.2-fold, about 3.3-fold, about 3.4-fold, about 3.5-fold, about 3.6-fold, about 3.7-fold, about 3.8-fold, about 3.9-fold, about 4.0-fold, about 4.1-fold, about 4.2-fold, about 4.3-fold, about 4.4-fold, about 4.5-fold, about 4.6-fold, about 4.7-fold, about 4.8-fold, about 4.9-fold, about 5.0-fold, about 5.1-fold, about 5.2-fold, about 5.3-fold, about 5.4-fold, about 5.5-fold, about 5.6-fold, about 5.7-fold, about 5.8-fold, about 5.9-fold, about 6.0-fold, about 6.1-fold, about 6.2-fold, about 6.3-fold, about 6.4-fold, about 6.5-fold, about 6.6-fold, about 6.7-fold, about 6.8-fold, about 6.9-fold, about 7.0-fold, about 7.1-fold, about 7.2-fold, about 7.3-fold, about 7.4-fold, about 7.5-fold, about 7.6-fold, about 7.7-fold, about 7.8-fold, about 7.9-fold, about 8.0-fold, about 8.1-fold, about 8.2-fold, about 8.3-fold, about 8.4-fold, about 8.5-fold, about 8.6-fold, about 8.7-fold, about 8.8-fold, about 8.9-fold, about 9.0-fold, about 9.1-fold, about 9.2-fold, about 9.3-fold, about 9.4-fold, about 9.5-fold, about 9.6-fold, about 9.7-fold, about 9.8-fold, about 9.9-fold, about 10.0-fold. about 10.1-fold, about 10.2-fold, about 10.3-fold, about 10.4-fold, about 10.5-fold, about 10.6-fold, about 10.7-fold, about 10.8-fold, about 10.9-fold, about 11.0-fold, about 11.1-fold, about 11.2-fold, about 11.3-fold, about 11.4-fold, about 11.5-fold, about 11.6-fold, about 11.7-fold, about 11.8-fold, about 11.9-fold, about 12.0-fold, about 12.1-fold, about 12.2-fold, about 12.3-fold, about 12.4-fold, about 12.5-fold, about 12.6-fold, about 12.7-fold, about 12.8-fold, about 12.9-fold, about 13.0-fold, about 13.1-fold, about 13.2-fold, about 13.3-fold, about 13.4-fold, about 13.5-fold, about 13.6-fold, about 13.7-fold, about 13.8-fold, about 13.9-fold, about 14.0-fold, about 14.1-fold, about 14.2-fold, about 14.3-fold, about 14.4-fold, about 14.5-fold, about 14.6-fold, about 14.7-fold, about 14.8-fold, about 14.9-fold, or about 15.0-fold.

In some implementations of the methods disclosed herein, heterologous expression of one or more of the HMGR enzymes disclosed herein in a transgenic or host cell may increase mevalonate production compared to the endogenous HMGR enzyme of the transgenic cell or host cell by 1.1-fold, 1.2-fold, 1.3-fold, 1.4-fold, 1.5-fold, 1.6-fold, 1.7-fold, 1.8-fold, 1.9-fold, 2.0-fold, 2.1-fold, 2.2-fold, 2.3-fold, 2.4-fold, 2.5-fold, 2.6-fold, 2.7-fold, 2.8-fold, 2.9-fold, 3.0-fold, 3.1-fold, 3.2-fold, 3.3-fold, 3.4-fold, 3.5-fold, 3.6-fold, 3.7-fold, 3.8-fold, 3.9-fold, 4.0-fold, 4.1-fold, 4.2-fold, 4.3-fold, 4.4-fold, 4.5-fold, 4.6-fold, 4.7-fold, 4.8-fold, 4.9-fold, 5.0-fold, 5.1-fold, 5.2-fold, 5.3-fold, 5.4-fold, 5.5-fold, 5.6-fold, 5.7-fold, 5.8-fold, 5.9-fold, 6.0-fold, 6.1-fold, 6.2-fold, 6.3-fold, 6.4-fold, 6.5-fold, 6.6-fold, 6.7-fold, 6.8-fold, 6.9-fold, 7.0-fold, 7.1-fold, 7.2-fold, 7.3-fold, 7.4-fold, 7.5-fold, 7.6-fold, 7.7-fold, 7.8-fold, 7.9-fold, 8.0-fold, 8.1-fold, 8.2-fold, 8.3-fold, 8.4-fold, 8.5-fold, 8.6-fold, 8.7-fold, 8.8-fold, 8.9-fold, 9.0-fold, 9.1-fold, 9.2-fold, 9.3-fold, 9.4-fold, 9.5-fold, 9.6-fold, 9.7-fold, 9.8-fold, 9.9-fold, 10.0-fold. 10.1-fold, 10.2-fold, 10.3-fold, 10.4-fold, 10.5-fold, 10.6-fold, 10.7-fold, 10.8-fold, 10.9-fold, 11.0-fold, 11.1-fold, 11.2-fold, 11.3-fold, 11.4-fold, 11.5-fold, 11.6-fold, 11.7-fold, 11.8-fold, 11.9-fold, 12.0-fold, 12.1-fold, 12.2-fold, 12.3-fold, 12.4-fold, 12.5-fold, 12.6-fold, 12.7-fold, 12.8-fold, 12.9-fold, 13.0-fold, 13.1-fold, 13.2-fold, 13.3-fold, 13.4-fold, 13.5-fold, 13.6-fold, 13.7-fold, 13.8-fold, 13.9-fold, 14.0-fold, 14.1-fold, 14.2-fold, 14.3-fold, 14.4-fold, 14.5-fold, 14.6-fold, 14.7-fold, 14.8-fold, 14.9-fold, or 15.0-fold.

The methods disclosed herein are the first to provide a process of bioproducing isoprenoids in batches that can be used for commercial consumption. This, the present disclosure provides batches of an isoprenoid (e.g., bakuchiol, farnesene, farnesol, geosmin, geraniol, terpineol, limonene, myrcene, linalool, hinokitiol, pinene, cafestol, kahweol, cembrene, taxadiene, α-bisabolol, α-guaiene, bergamontene, and valencene etc.) produced by the methods disclosed herein. A bioproduction batch of an isoprenoid (e.g., bakuchiol, farnesene, farnesol, geosmin, geraniol, terpineol, limonene, myrcene, linalool, hinokitiol, pinene, cafestol, kahweol, cembrene, taxadiene, α-bisabolol, α-guaiene, bergamontene, and valencene etc.) may have a chemical purity of at least 90%, at least 91%, at least 92%, at least 93%, at least 94%, at least 95%, at least 96%, at least 97%, at least 98%, at least 99%, or 100%, or any values in between any of the two aforementioned values, and no single impurity of greater than 1%, no greater than about 0.5%, or greater than about 0.1%. The level of impurities in a given batch can be determined by high-performance liquid chromatography (HPLC) and other suitable techniques.

### Non-Limiting Working Examples

The following examples are given to illustrate the present disclosure. It should be understood, however, that the disclosure is not to be limited to the specific conditions or details described in these examples.

### I. Example 1—Identification and Assessment of HMGR Enzymes

Blast searches and Hmm searches were performed to identify putative heterologous HMGR enzymes. All search hits were annotated using Blast against the public nr database. Results were trimmed and organized for further assessment.

All genes encoding putative heterologous HMGRs were integrated into S. cerevisiae via standard LiAc chemical transformation methodologies using a Cas12-based system for directed nuclease-guided genomic integration. HMGR genes were expressed from the GTT2 locus, driven by a TDH3 promoter and ADH1 terminator.

Resulting strains were grown and assayed at 30° C. in 96 mid-well plates with 3% maltodextrin, 0.2% glucose defined media (modified from Westfall 2012) with alpha-amylase for 24-48 hours, before transfer to the same media for 48 hours.

For farnesene detection, samples were diluted with butanol, and analyzed on an Agilent 1290 LC-MS system using a triple quadrupole 6470 mass spectrometer. A Phenomenex 1.1 micron Kinetic 2.1×30 mm C18 column was used, with 0.1% formic acid in as mobile phase A, and 0.1% formic acid in acetonitrile in mobile phase B. A rapid gradient method was run with a dual injector system for a total peak to peak time of 0.36 minutes. Detection was at 220 nm with a diode array detector. Data were quantitated with MassHunter, and normalized to on-plate positive and negative control strains. External standard curves of trans-beta farnesene were used for absolute quantitation.

For mevalonate, samples were detected using a reversed-phase LCMS method with a PFP column via a specific MRM tuned on an authentic standard of the analyte.

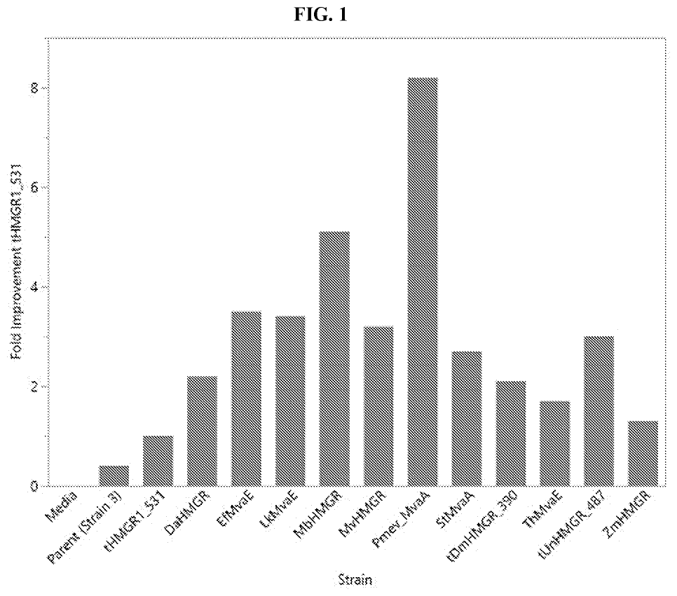

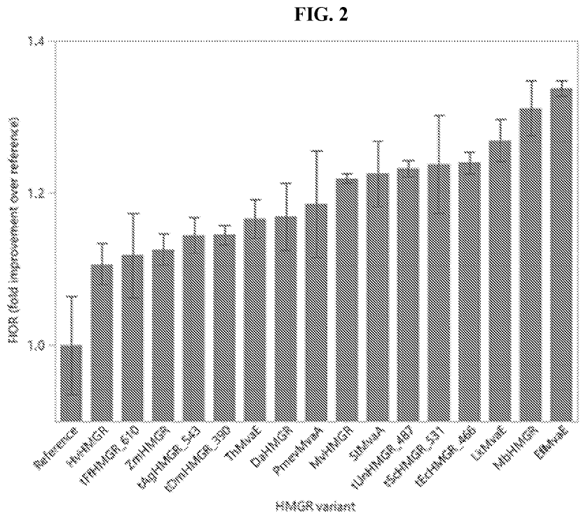

Wild type S. cerevisiae strains that included one of the identified heterologous genes and expressed a heterologous HGMR showed up to 8.2× improvement in mevalonate production as assayed by both an intra and extracellular metabolite assay using targeted LCMS methodologies (FIG. 1). The HMGR from Pseudomonas mevalonii MvaA (i.e., Pmev_MvaA SEQ ID NO: 13) resulted in the highest level of mevalonate production that was 8.2× the amount produced by the control strain expressing tHMGR_531 (SEQ ID NO: 6), and most of the HMGRs that were tested improved mevalonate production by at least 2× compared to tHMGR_531. Strains expressing heterologous HMGRs from Tetragenococcus halophilus MvaE (i.e., ThMvaE; SEQ ID NO: 15) and Zea mays HMGR (i.e., ZmHMGR; SEQ ID NO: SEQ ID NO: 16) did not increase production by more than 2×, but still produced more mevalonate than the control strain expressing tHMGR_531. Strains containing a farnesene synthase were also tested to assay terpene productivity. Several yeast strains were engineered to express one of 15 heterologous HMGR enzymes, and all 15 heterologs provided for increased production of the isoprenoid product farnesene (FIG. 2) compared to a reference strain of S. cerevisiae that did not express any heterologous HMGR enzymes. Thus, expression of any of the heterologous HMGRs disclosed herein appears sufficient to increase production of a desired isoprenoid, such as farnesene.

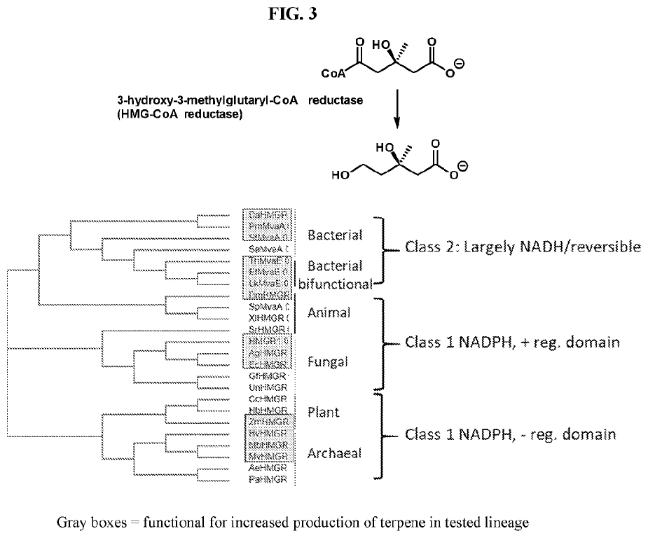

Novel enzymes identified include but are not limited to: Delftia acidovorans HMGR (SEQ ID NO: 8), Haloferax volcanii HMGR (SEQ ID NO: 9), Lactobacillus Koumiss MvaE (SEQ ID NO: 10), Methanococcoides burtonii HMGR (SEQ ID NO: 11), Methanosarcina lacusiris HMGR (SEQ ID NO: 12), Pseudomonas mevalonii MvaA (SEQ ID NO: 13), Streptococcus thermophilus MvaA (SEQ ID NO: 14), Tetragenococcus halophilus MvaE (SEQ ID NO: 15), and Zea mays HMGR (SEQ ID NO: SEQ ID NO: 16). Engineered enzymes include but are not limited to: Ashbya gossypii truncated HMGR (SEQ ID NO: 1), Drosophila melanogaster truncated HMGR (SEQ ID NO: 2), Eremothecium cymbalariae truncated HMGR (SEQ ID NO: 3), Fusarium fujikuroi truncated HMGR (SEQ ID NO: 4), and Uncinula necator truncated HMGR (SEQ ID NO: 5). As can be seen in FIG. 3, the HMGR enzymes disclosed and characterized herein lie across a diversity of kingdoms, thus showing that heterologous expression within a yeast expression system is not only possible, but effective in increase mevalonate flux and ultimately increasing production of a desired isoprenoid.

Truncations were made manually using secondary structure predictions from TOPCONS (topcons.cbr.su.se/pred/) and amino acid alignments.

It should be appreciated that all combinations of the disclosed concepts are contemplated as being part of the inventive subject matter disclosed herein and may be employed in any combination to achieve the benefits described herein.

The present technology is not to be limited in terms of the particular implementations described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular implementations only, and is not intended to be limiting.

All patents and publications disclosed herein are herein incorporated by reference to the same extent as if each individual publication was specifically and individually indicated to be incorporated by reference.

1. An isolated, truncated enzyme, comprising an amino acid sequence with at least about 90% identity with any one of SEQ ID NOs: 1 (tAgHMGR_543), 2 (tDmHMGR_390), 3 (tEcHMGR_466), 4 (tFfHMGR_610), and 5 (tUnHMGR_487), or a variant thereof with up to 20 amino acids deleted from the N-terminus; or an amino acid sequence with at least about 90% identity but less than 100% identity with SEQ ID NO: 6 (tHMGR 531), or a variant thereof with up to 20 amino acids deleted from the N-terminus.

2-14. (canceled)

15. A nucleic acid comprising a nucleic acid sequence encoding the isolated enzyme of claim 1.

16. A transgenic cell, comprising a first nucleic acid encoding a first heterologous 3-hydroxy-3-methylglutaryl-coenzyme A reductase (HMGR) enzyme that (i) lacks an inhibitory domain, (ii) utilizes NAD or NADP as a cofactor, or (iii) a combination thereof.

17. The transgenic cell of claim 16, wherein the transgenic cell comprises only one copy of the first nucleic acid.

18. The transgenic cell of claim 16 further comprising a second, third, fourth, or fifth nucleic acid encoding a second, third, fourth, or fifth heterologous HMGR enzyme that (i) lacks an inhibitory domain, (ii) utilizes NAD or NADP as a cofactor, or (iii) a combination thereof.

19. The transgenic cell of claim 18, wherein the transgenic cell comprises only one copy of each of the second, third, fourth, or fifth nucleic acid.

20. The transgenic cell of claim 18, wherein the first, second, third, fourth, or fifth nucleic acid each independently comprises a different nucleic acid sequence or encodes a different heterologous HMGR enzyme.

21. The transgenic cell of claim 16, wherein the heterologous HMGR enzyme increases flux to mevalonate compared to a native yeast HMGR enzyme.

22. The transgenic cell of claim 16, wherein the transgenic cell does not comprise multiple copies of nucleic acid sequences encoding a native yeast HMGR enzyme.

23. The transgenic cell of claim 16, wherein the first heterologous HMGR enzyme comprises an amino acid sequence with at least about 90% identity with any one of SEQ ID NOs: 1-5 or 8-16.

24-52. (canceled)

53. The transgenic cell of claim 16 further comprising a single copy of a second nucleic acid encoding a second, different heterologous HMGR enzyme that comprises an amino acid sequence with at least about 90% identity with any one of SEQ ID NOs: 1-16.

54. (canceled)

55. The transgenic cell of claim 53 further comprising a single copy of a third nucleic acid encoding a third, different heterologous HMGR enzyme that comprises an amino acid sequence with at least about 90% identity with any one of SEQ ID NOs: 1-16.

56-82. (canceled)

83. A method of producing an isoprenoid, comprising culturing the transgenic cell according to claim 16.

84. The method of claim 83, wherein the isoprenoid is a sesquiterpene, a monoterpene, a diterpene, or a meroterpene.

85. The method of claim 83, wherein the isoprenoid is selected from bakuchiol, farnesene, farnesol, geosmin, geraniol, terpineol, limonene, myrcene, linalool, hinokitiol, pinene, cafestol, kahweol, cembrene, taxadiene, α-bisabolol, α-guaiene, bergamontene, and valencene.

86-104. (canceled)

105. The transgenic cell of claim 16, wherein the transgenic cell is Saccharomyces cerevisiae (S. cerevisiae).

106. The transgenic cell of claim 16, wherein the first nucleic acid is integrated into the transgenic cell's genome.

107. The transgenic cell of claim 16, wherein the first nucleic acid is not integrated into the transgenic cell's genome.

108. The transgenic cell of claim 16, wherein the first heterologous HMGR enzyme comprises an amino acid sequence with at least about 90% identity to SEQ ID NO: 7.

109. The transgenic cell of claim 105, wherein the first heterologous HMGR enzyme comprises an amino acid sequence with at least about 90% identity to SEQ ID NO: 7.


SEQUENCE LISTING (32 sequences)

SEQ ID NO: 1 - protein, 486 aa, unidentified
MARETIPKSTVSSSETKVVGSVASSIVPSDDETETEDEAEPVRPLATLIDVLRKGAVKTLKNKEVVSLVVNSELPLYALEKQLGDTTRAVIVRRKALAKLADAPVLETERLPYKHYDYDRVFGACCENVIGYMPLPVGVIGPLVIDGVAYHIPMATTEGCLVASAMRGCKAINAGGGVTTVLTKDGMTRGPCVRFPSLARAGACKLWLDSEEGQARVKRAFNSTSRFARLQHVQTALAGDLLFIRFRTTTGDAMGMNMISKGVEFALHQMGAEFGWHDMEIVAVSGNYCTDKKPAAINWIEGRGKSVVAEATVPADVVRKVLKSDVAALVDVNISKNLVGSAMAGAVGGFNAHASNLVTALYLALGQDPAQNVESSNCITLMRDVGGDLRVSVSMPSIEVGTIGGGTILGPQSAMLDLLGVRGPHPSAPGTNARQLAKIVASAVLAGELSLCSALAAGHLVQSHMIHNRAKTPADPEVPCRRPACI

SEQ ID NO: 2 - protein, 530 aa, unidentified
MDQLRQSGPVAIAAKASQTTPIDEEHVEQEKDTENSAAVRTLLFTIEDQSSANASTQTDLLPLRHRLVGPIKPPRPVQECLDILNSTEEGSGPAALSDEEIVSIVHAGGTHCPLHKIESVLDDPERGVRIRRQIIGSRAKMPVGRLDVLPYEHFDYRKVLNACCENVLGYVPIPVGYAGPLLLDGETYYVPMATTEGALVASTNRGCKALSVRGVRSVVEDVGMTRAPCVRFPSVARAAEAKSWIENDENYRVVKTEFDSTSRFGRLKDCHIAMDGPQLYIRFVAITGDAMGMNMVSKGAEMALRRIQLQFPDMQIISLSGNFCCDKKPAAINWIKGRGKRVVTECTISAATLRSVLKTDAKTLVECNKLKNMGGSAMAGSIGGNNAHAANMVTAVFLATGQDPAQNVTSSNCSTAMECWAENSEDLYMTCTMPSLEVGTVGGGTGLPGQSACLEMLGVRGAHATRPGDNAKKLAQIVCATVMAGELSLMAALVNSDLVKSHMRHNRSSIAVNSANNPLNVTVSSCSTIS

SEQ ID NO: 3 - protein, 577 aa, unidentified
MKVYFQLEDTILSSLRYVSVAIRDRFISKLVLFALAISASINIYLLNIARIHTQFTTNELNSKKKLKKSSNFAVGSAPIVAPPSERTSESTVSSSETKIMDSVPSSVTVSDDETETEDESEPIRPLETLIEIMKQGGVKTLRNRELVSLIVNSELPLYALEKQLCDTTRAVVVRRKALAKLADAPALETERLPYKNYDYDRVFGACCENVIGYMPLPVGVIGPLVIDGIPYHIPMATTEGCLVASAMRGCKAINAGGGVTTVLTKDGMTRGPCIRFPSLARSGACKIWLDSEDGQNKIKKAFNSTSRFARLQHIQTALAGDLLFIRFRTTTGDAMGMNMISKGVEFSLNQMVEEFGWDDMEIVAVSGNYCTDKKPAAINWIEGRGKSVVAEATIPGDVVKKVLKSDVNALVDLNISKNLIGSAMAGSVGGFNAHASNLVTAVYLALGQDPAQNVESSNCMTLMKEIDGDLRISVSMPSIEVGTIGGGTILEPQSAMLDLLGVRGPHPTEPGTNARQLAKVVACAVMAGELSLCSALAAGHLVQSHMIHNRAKASPTSTEVKQDDIPRLQEGSVTCIK

SEQ ID NO: 4 - protein, 572 aa, unidentified
MVLSKWIVIALALSVALNGYLFNVARWGIKDPNVPEHNIDRNELARAQQFNDTGSATLPLGEYVPPTPMRTQPSTPAITDDEAEGLHMTKARPANLPNRSNEELEKLLSEKRVREMTDEEVISLSMRGKIPGYALEKTLGDFTRAVKIRRSIIARNKATTDITHSLDRSKLPYENYNWERVFGACCENVIGYMPLPVGVAGPLVIDGQSYFIPMATTEGVLVASASRGCKAINSGGGAITVLTADGMTRGPCVAFETLERAGAAKLWLDSEAGQDMMKKAFNSTSRFARLQSMKTALAGTNLYIRFKTTTGDAMGMNMISKGVEHALSVMANDGGFDDMQIISVSGNYCTDKKAAALNWIDGRGKGVVAEAIIPGEVVRSVLKSDVDSLVELNVAKNLIGSAMAGSVGGFNAHAANIVAAIFLATGQDPAQVVESANCITIMKNLNGALQISVSMPSLEVGTLGGGTILEPQGAMLDILGVRGSHPTNPGDNARRLARIIGAAVLAGELSLCSALAAGHLVRAHMQHNRSAAPSRSTTPAPPMTPVSLAMTSAQERSASTTSMSAAAIQRSK

SEQ ID NO: 5 - protein, 521 aa, unidentified
MSTIKYHQNEASDVISSSPQKKSQFMETNQPYDNTLQTPINIDDEEEGLEICLSKSKSPSKRTQAQLEMMLKENQASELDDQELIELSLQGKIPGYALEKKLKDTTRAVKIRRAVISRTLTTSQTTGLLEYSKLPYKNYDWDRVLGACCENVIGYMPLPLGVAGPIIIDSQSYFIPMATTEGVLVASTSRGAKAINAGGGAVTVITGDGMTRGPCVSFETLERAGAAKVWLDSEIGQKIITKAFNSTSRFARLQSIKTALAGTYLYPRFKTTTGDAMGMNMISKGVEHALNVMATEAGFEDMQIISVSGNFCTDKKPAAINWIDGRGKSVVAEAIIPKDIVKSVLKSTVDAMVELNISKNLVGSAMAGSIGGFNAHAANIVTAIFLATGQDPAQNVESSNCITLMRNLGGNLQISVSMPSIEVGTLGGGTILEPQGAMLDMLGVRGSHPTHPGENARRLARIIAASVLSGELSLCSALAAGHLVKSHMAHNRSAPITRSNTPAQISTHPSMISTNSMREKH

SEQ ID NO: 6 - protein, 527 aa, unidentified
MAADQLVKTEVTKKSFTAPVQKASTPVLTNKTVISGSKVKSLSSAQSSSSGPSSSSEEDDSRDIESLDKKIRPLEELEALLSSGNTKQLKNKEVAALVIHGKLPLYALEKKLGDTTRAVAVRRKALSILAEAPVLASDRLPYKNYDYDRVFGACCENVIGYMPLPVGVIGPLVIDGTSYHIPMATTEGCLVASAMRGCKAINAGGGATTVLTKDGMTRGPVVRFPTLKRSGACKIWLDSEEGQNAIKKAFNSTSRFARLQHIQTCLAGDLLFMRFRTTTGDAMGMNMISKGVEYSLKQMVEEYGWEDMEVVSVSGNYCTDKKPAAINWIEGRGKSVVAEATIPGDVVRKVLKSDVSALVELNIAKNLVGSAMAGSVGGFNAHAANLVTAVFLALGQDPAQNVESSNCITLMKEVDGDLRISVSMPSIEVGTIGGGTVLEPQGAMLDLLGVRGPHATAPGTNARQLARIVACAVLAGELSLCAALAAGHLVQSHMTHNRKPAEPTKPNNLDATDINRLKDGSVTCIKS

SEQ ID NO: 7 - protein, 803 aa, unidentified
MKTVVIIDALRTPIGKYKGSLSQVSAVDLGTHVTTQLLKRHSTISEEIDQVIFGNVLQAGNGQNPARQIAINSGLSHEIPAMTVNEVCGSGMKAVILAKQLIQLGEAEVLIAGGIENMSQAPKLQRFNYETESYDAPFSSMMYDGLTDAFSGQAMGLTAENVAEKYHVTREEQDQFSVHSQLKAAQAQAEGIFADEIAPLEVSGTLVEKDEGIRPNSSVEKLGTLKTVFKEDGTVTAGNASTINDGASALIIASQEYAEAHGLPYLAIIRDSVEVGIDPAYMGISPIKAIQKLLARNQLTTEEIDLYEINEAFAATSIVVQRELALPEEKVNIYGGGISLGHAIGATGARLLTSLSYQLNQKEKKYGVASLCIGGGLGLAMLLERPQQKKNSRFYQMSPEERLASLLNEGQISADTKKEFENTALSSQIANHMIENQISETEVPMGVGLHLTVDETDYLVPMATEEPSVIAALSNGAKIAQGFKTVNQQRLMRGQIVFYDVADAESLIDELQVRETEIFQQAELSYPSIVKRGGGLRDLQYRAFDESFVSVDFLVDVKDAMGANIVNAMLEGVAELFREWFAEQKILFSILSNYATESVVTMKTAIPVSRLSKGSNGREIAEKIVLASRYASLDPYRAVTHNKGIMNGIEAVVLATGNDTRAVSASCHAFAVKEGRYQGLTSWTLDGEQLIGEISVPLALATVGGATKVLPKSQAAADLLAVTDAKELSRVVAAVGLAQNLAALRALVSEGIQKGHMALQARSLAMTVGATGKEVEAVAQQLKRQKTMNQDRALAILNDLRKQ

SEQ ID NO: 8 - protein, 429 aa, unidentified
MVADSRLPNFRALTPAQRRDFLADACGLSDAERALLAAPGALPLALADGMIENVFGSFELPLGVAGNFRVNGRDVLVPMAVEEPSVVAAASYMAKLAREDGGFQTSSTLPLMRAQVQVLGVTDPHGARLAVLQARAQIIERANSRDKVLIGLGGGCKDIEVHVFPDTPRGPMLVVHLIVDVRDAMGANTVNTMAESVAPLVEKITGGSVRLRILSNLADLRLARARVRLTPQTLATQDRSGEEIIEGVLDAYTFAAIDPYRAATHNKGIMNGIDPVIVATGNDWRAVEAGAHAYASRSGSYTSLTRWEKDAGGALVGSIELPMPVGLVGGATKTHPLARLALKIMDLQSAQQLGEIAAAVGLAQNLGALRALATEGIQRGHMALHARNIALVAGATGDEVDAVARQLAAEHDVRTDRALEVLAALRARA

SEQ ID NO: 9 - protein, 403 aa, unidentified
MTDAASLADRVREGDLRLHELEAHADADTAAEARRLLVESQSGASLDAVGNYGFPAEAAESAIENMVGSIQVPMGVAGPVSVDGGSVAGEKYLPLATTEGALLASVNRGCSVINSAGGATARVLKSGMTRAPVFRVADVAEAEALVSWTRDNFAALKEAAEETTNHGELLDVTPYVVGNSVYLRFRYDTKDAMGMNMATIATEAVCGVVEAETAASLVALSGNLCSDKKPAAINAVEGRGRSVTADVRIPREVVEERLHTTPEAVAELNTRKNLVGSAKAASLGFNAHVANVVAAMFLATGQDEAQVVEGANAITTAEVQDGDLYVSVSIASLEVGTVGGGTKLPTQSEGLDILGVSGGGDPAGSNADALAECIAVGSLAGELSLLSALASRHLSSAHAELGR

SEQ ID NO: 10 - protein, 812 aa, unidentified
MKEVVIIDAARTPIGKYKGSLSSFSAVELGTMVTKKLLEKASIKKDEINQVIFGNVLQAGNGQNVARQISIISDIPVDVPAMTINEVCGSGMKAVILARQLIQLGEADLVIAGGTESMTRAPLLQQFDSETTSYNGPISSMVNDGLTDTFSNTHMGLTAENVAEQFGVTRKEQDQYALDSQLKAAKATENNVFKEEIIPVTLPDGTLLENDEAIRGNSSLEKLGTLKTVFSENGTVTAGNASPLNDGASVMILASKEYALKNDLPYLATIKGVAEIGIDPSIMGIAPINAINSLLEKTDVSLDAIDRFEINEAFAASSIVVNRELQLDPEKVNSDGGAIALGHPIGASGARILTTLSYGLQRNEQKYGIASLCIGGGLGLAVLLEANQEKAGSFNEKKKFYQLTPEERRSQLVRGGVISKESADQLKNERLSEDIANHLIENQISQVEIPMGVAQNFQINGEKKWVPMATEEPSVIAAASNGAKICGNITAKTPQRLMRGQIVLTGKSEYQAIIEAIDTRKDELFLCANNSYPSIVKRGGGVRDISTREFMGSDHAYVSIDFLIDVKDAMGANIVNAILEGVASQLRSWFPDEEILFSILSNLATESLATACCTIPFEYLGKSKEAGRQVAEKIQQAAEYAKLDVYRAATHNKGIMNGIEAVILATGNDTRAASAAIHAYASRNGFYQGLTDWKIVDGQLVGKLTVPLAVATVGGASKILPKAKLALEILDVSSAKELAQVIAAVGLAQNLAALKALVTEGIQKGHMSLQARALAITVGATGDEIEQVASYLRKADTMNQQLASDYLLETRS

SEQ ID NO: 11 - protein, 414 aa, unidentified
MASKTETTMKEDELLEKVVSGEMPLRKIDAYTDTDTAVRVRKCAIEKMNGVKFEHIQNYTIDAEAATKRNIENMIGTIQIPLGVAGAIMVNGEYASGEFMLPLATTEGALVASVNRGCTVITASGGSNVRIFQDLMTRAPVFKLENVNKVKEFVDWVKREETFTNMKEKAGETTRFGELLSVDPFITGNTVFLRFAYDTKDAMGMNMVTIATDAVLNFISEDFGVYPISLSGNMCTDKKPAAINNILGRGKTVAADVTIPKEIVEKKLKTTPKMMEEVNYRKNLLGSARAGALGFNAHAANIIAALYLACGQDAAHVVEGSSAITTMEVNENGDLYCSVTLPSIQVGTVGGGTGIATQRDCLNLLGVAGAGEVPGHNSKKLAEIIAAAVLAGEISLIGAQAAGHLAKAHAELGR

SEQ ID NO: 12 - protein, 418 aa, unidentified
MFLKDNDLTEDEKLLLQKVLDGDIAFRKIEEFADPLTAVKIRRLAIQEYAKLEFEHIQNFSLDVETVTKKNIENMIGAVQIPLGVAGLLKVNGEYADAEYYIPLATTEGALVASVNRGCSVITKSGGANVRVFEDEMTRAPVFKLESLDRTKKFYEWVKSPEIFEQMKTVAEKTTRFGKLLSVKPFVTGTYVYLRFSYDTKDAMGMNMVTIATDAVMHLIEDEFGAHPITLSGNMCTDKKPASISTILGRGKTVVAEVTIPEEIVKETLKCTPDAMFEVNYSKNLLGSARAGALGFNAHAANVIAAVYLACGQDAAHVVEGSTAITSMELTKYGEIHCSVTLPALPVGTVGGGTGLGTQRDCLNILGVAGTGDIPGINSRKFAEIVASAVLAGEISLIGAQAAGHLARAHAQLGRGKF

SEQ ID NO: 13 - protein, 428 aa, unidentified
MSLDSRLPAFRNLSPAARLDHIGQLLGLSHDDVSLLANAGALPMDIANGMIENVIGTFELPYAVASNFQINGRDVLVPLVVEEPSIVAAASYMAKLARANGGFTTSSSAPLMHAQVQIVGIQDPLNARLSLLRRKDEIIELANRKDQLLNSLGGGCRDIEVHTFADTPRGPMLVAHLIVDVRDAMGANTVNTMAEAVAPLMEAITGGQVRLRILSNLADLRLARAQVRITPQQLETAEFSGEAVIEGILDAYAFAAVDPYRAATHNKGIMNGIDPLIVATGNDWRAVEAGAHAYACRSGHYGSLTTWEKDNNGHLVGTLEMPMPVGLVGGATKTHPLAQLSLRILGVKTAQALAEIAVAVGLAQNLGAMRALATEGIQRGHMALHARNIAVVAGARGDEVDWVARQLVEYHDVRADRAVALLKQKRGQ

SEQ ID NO: 14 - protein, 423 aa, unidentified
MTKLSWTGFSKKTLQERKEHLKNNALLSQENQDLLDNDQQLTLETANQMAENVIGRFTLPFAICPDVLVDGVTYQVPMVTEEPSVVAAASYASKLIKRSGGFTTKIHDRQMIGQVALFDVPDKATAASKIQAASQKLIDIAKEAYPSIVKRGGGPRKLWTETKGDFLIVYLAVDTQEAMGANMVNTMMEALVPELENLSEGQSLMAILSNLATESLVTATCRLNTRFLSRNKAEAHNFAKKMELASQLAQVDPYRAATHNKGIFNGIDALVIATGNDWRAVEAGCHAYASKDGSYRGLSTWTYNQETKELVGELTLPMPIATRGGSIGLNPSVSIAHDLLNHPDARTLAGIIVSLGLVQNLAALKALTSTGIQAGHMKLQAKSLALLAGANPEEMPHVLSELLKAKHMNQETAQAILEKLRNP

SEQ ID NO: 15 - protein, 808 aa, unidentified
MKDVVIIDALRTPVGKYQGSLSQLSAVELGSAVSKKLINNNKKAAAAINQVIFGNVLQAGSGQNPARQITLNSGLSESVYASTINEVCGSGMKAISLASQAIFLDEAEVVLAGGTESMSQAPYLSYYNQQEDTYSQPKPAMLSDGLTDVFSGQHMGLTAENVAEKFNITRKMQDAFALRSQERAANAQEKGYFSNEILPLDIAGKKVDKDEGVRKDTSLEKLAKLKTVFKKEGTVTAGNASTINDGASAVLLASKNFALANDLSYLAVLKDVVEVGVDPKVMGISPIKAIRQLLERNALAIENIDLFEINEAFASSSIAVEQELEIPEDKVNVCGSGISIGHAIGASGARIITTACHQLERVDGRYAVVSLCVGGGLGLAALIERPKANKSHKFYQLTRKERLDFLVSHNKITSKTVDELERTVLPESIAGNLTENQMSEISLPMGLVSNMSVNQKDYFVPMATEEPSVVAACNNGVQMAKSSGGFTAVMKKKEIRGQIVLMNVTDKSTVIEQIEKNEAEIISTAEQSYPSIVKRGGGVKRVVVREFAEDPNFLSVDLIVDTQDAMGANMLNTMLEAVATLFRQWFSEEILFSILSNYATDALVSAECYISFASLGKGDAEKGEKIAEKIAAASNFAQIDPFRAATHNKGIMNGIDAVVLATGNDTRSVNSAVHAYAAKNGKYQGLSQWEIVDNQLKGSIELPLAVATAGGATKVLPKAQAALQILDVNDAKELAEVIASVGLAQNLAALKALVTEGIQKGHMALQARTLALSVGAKDSEVQKVANRLKRQQMNEENARKILQELRNR

SEQ ID NO: 16 - protein, 579 aa, unidentified
MEVRGGVGQGSAARHPPAPEPSRAAARVQAGDALPLPIRHTNLIFSALFAASLAYLMRRWREKIRSSTPLHAVGLAEMLAIFGLVASLIYLLSFFGIAFVQSIVSSGDDDEDFLVGSGSSGSAAAPSRQHAQAPAPCELLGSPAAAPEKMPEDDEEIVASVVAGKVPSYALEARLGDCRRAAGIRREALRRITGRDIEGLPLDGFDYASILGQCCELPVGYVQLPVGVAGPLLLDGRRFYLPMATTEGCLVASTNRGCKAIAESGGATSVVLRDAMTRAPVARFPTARRAAELKAFLEDPANFDTLSVVFNRSSRFARLQGVQCAMAGRNLYMRFSCSTGDAMGMNMVSKGVQNVLDFLQDDFHDMDVISISGNFCSDKKPSAVNWIEGRGKSVVCEAVIGEEVVKKVLKTDVQSLVELNTIKNLAGSAVAGALGGFNAHASNIVTAIFIATGQDPAQNVESSHCITMLEPVNAGRDLHISVTMPSIEVGTVGGGTQLASQSACLDLLGVRGASRDRPGSNARLLATVVAGGVLAGELSLLSALAAGQLVKSHMKYNRSSKDVSSTTATEKTRQREVDV

SEQ ID NO: 17 - DNA, 1461 bp, unidentified
  source 1..1461: mol_type = other DNA
atggcacgtgaaactattccaaaatctacggtttcgtcttctgaaactaaagtggttggctctgtagcctcttcaatcgtcccaagcgacgatgaaaccgaaacggaagatgaagcggaacctgtcagaccattagcaactttaatcgacgtcttgaggaagggggctgtgaaaacattgaagaacaaagaagttgtttctcttgttgttaactctgaactaccactatatgctttagaaaagcaattaggagacacgactagggcagttattgtcagaagaaaggctttggcaaagttagctgatgcacctgttctggaaacggaaagattgccttataaacattacgactatgatagagtcttcggtgcgtgttgcgaaaacgttatcgggtatatgccattgcccgttggcgtaattggccccttagtcattgacggtgttgcctaccatatcccaatggcaactactgaaggttgtttggtcgcatccgctatgagaggatgtaaagctataaacgcaggtggaggtgtaaccactgtcttaacaaaggatggcatgaccagagggccatgcgtcagatttccatcgctggcgagggcgggtgcttgtaagttgtggctagatagtgaagaggggcaggcaagagtcaaacgtgctttcaattccaccagcagattcgctcgtctgcaacacgttcagacggctttagccggtgatttgttgttcattaggtttaggactacaactggagacgctatggggatgaacatgattagtaaaggtgtcgaatttgcattgcatcaaatgggcgcggaatttggttggcatgatatggaaattgttgctgtttctggaaattattgtacggataagaaacccgcagccataaattggatagagggtagaggtaaaagcgttgtcgctgaggcaaccgtgcctgcagacgtcgtgagaaaagtattgaagtctgacgtcgccgcattggttgatgtcaatatatccaaaaatttagtaggctctgcaatggcgggagctgttggtggtttcaacgcacacgctagtaaccttgtcacagccttgtacctggctctaggtcaagacccagcccaaaatgttgaaagttccaattgcataacactgatgagggatgtgggtggagacctaagagtttcggtaagcatgccaagtatagaagttgggaccatcggtggaggaacaattttaggtccccagtcggcaatgcttgacttattaggtgtcagaggtcctcatccaagcgcccctggtacaaatgcaagacagttggctaaaattgttgcttctgctgttcttgctggcgaactttctctgtgtagcgccttagctgctggacatctagtccaatcccacatgattcataacagagcaaaaacgccagcagatccggaagttccatgtaggagacccgcatgtatttga

SEQ ID NO: 18 - DNA, 1593 bp, unidentified
  source 1..1593: mol_type = other DNA
atggaccagttaaggcaatccgggccagttgcgatagccgctaaggcttcacaaacgactccaattgacgaggaacatgtggaacaagaaaaagacacggaaaattcagcggctgttaggactttgctatttactatcgaagatcaatcttctgcaaatgcatctacgcaaactgatttgttgcccttgcgtcatagattggtcggaccgattaaaccacctagaccagtacaagaatgccttgatatactgaacagtactgaagaggggtccgggcctgcggctctttcagatgaagaaatcgtatcaattgttcatgctggaggaactcattgtccattacataagattgaatcagtattagatgacccggaaaggggcgtgaggataagaaggcaaataatcggtagtcgtgcaaaaatgcctgttggtagactagatgttctaccatacgaacacttcgactatagaaaggtcttaaacgcctgttgcgaaaacgtattgggttacgtgccaatcccagttggttatgcaggtccattgttattagatggtgaaacatattatgtacctatggcgactacggagggagctctagttgcatccactaataggggttgtaaggcattgagcgtaagaggagtaagatccgtggtcgaagatgtaggcatgaccagagcaccctgcgtcaggtttccttcagtagcaagagcagctgaagctaagtcttggatagaaaatgatgaaaactatagagtagtcaaaacagaatttgattctaccagtagatttggtcgtctgaaagattgtcacatagctatggatgggccacaattatacattagattcgtagctattactggtgatgcaatgggtatgaacatggtgtccaaaggcgctgaaatggcacttagaaggatccaattgcaatttccggatatgcaaattatatccttgtcaggtaatttttgctgcgataaaaagccagccgcaataaattggataaagggaagaggtaaaagggttgttactgaatgtaccatatctgcagcaactctgaggtcagtcttaaagactgatgctaaaacgctagttgaatgtaataagttaaaaaatatgggaggcagtgctatggcaggatcaataggaggaaacaatgcacatgctgcaaatatggttactgctgtatttttggctacaggtcaagatcctgcacagaatgttacttcgagcaattgctcaaccgctatggagtgttgggctgaaaattccgaagatttatacatgacttgtaccatgccaagcttggaagtcggtacggtaggtggaggtaccggcttgccaggtcagagtgcatgcctagaaatgctgggtgtacgtggcgctcatgcgactagacccggtgataatgcaaaaaagttggcgcaaatcgtctgcgctactgttatggctggagaattgagtttaatggcagcactggtaaactctgatttagttaaatctcatatgagacataacagatcttctatagctgtgaactctgctaataatcctcttaatgtaaccgtctcatcttgttctacgatttcttaa

SEQ ID NO: 19 - DNA, 1734 bp, unidentified
  source 1..1734: mol_type = other DNA
atgaaggtctattttcaattagaggatactatcctttcgagtctaaggtatgtgtcggttgccatcagagatagattcattagtaaattagtactttttgctctagctatcagtgcgtctatcaacatatatctgttgaacatagctagaattcacactcaattcacgaccaatgaacttaatagtaagaaaaaattgaagaaatctagtaatttcgcagttggcagtgctccaatagttgctccaccatcggaaagaaccagcgaatctacagtatcatcctcagaaactaaaataatggactcagtcccttcgtctgtcaccgtctctgatgacgaaactgaaactgaagatgaatccgaacccattagaccgttggaaactttaattgaaattatgaaacagggtggagttaaaactttgagaaacagagaattagtctccttgatcgttaatagtgaactgcccctgtacgcattagaaaagcaactatgtgataccacaagggccgtcgttgttagaagaaaagcattggccaaacttgcagatgctccagcgttagaaactgaacgtctaccatacaaaaattatgactatgatagggtttttggagcttgctgtgaaaatgttatcggttatatgccccttcccgttggggtcattggacctctggttatcgatggtataccttatcacataccaatggcaactacagaaggttgtttagtagcctcggcgatgagaggttgcaaggcaataaatgccggaggaggggtgacaacagtattgactaaggatggaatgacaaggggtccatgcattagattcccttctttggcaagatcaggtgcttgtaagatttggttagactctgaggatggtcaaaataaaataaagaaagcatttaactctacatccagatttgctcgtctacagcatattcaaactgcgttagcgggcgatcttttattcattagattcagaaccactacgggtgatgcgatgggtatgaatatgatctccaagggcgtagaattcagccttaatcagatggtggaggagttcgggtgggacgacatggaaatagtagccgtgtccggtaactattgcacagacaaaaagccagcggcaatcaattggattgaaggaagaggtaaatcagtggtagctgaggcaactattccaggggatgtcgtaaagaaagttcttaagtctgacgtcaatgctctagtcgaccttaatattagcaaaaatttaatcggtagtgctatggctggtagtgttgggggatttaatgctcatgcctccaacttagtcacagcagtttacctagctctaggacaggatccagcccaaaatgttgaatcctctaactgtatgacattaatgaaagaaattgacggtgacttaagaataagcgtcagtatgccttctatagaagttggtacgatcggtggtggtacaatattggaaccccagtcagctatgttggatttattgggagtgagaggcccccatcctactgagcctggcaccaacgcaaggcaattagctaaagttgtggcctgcgccgtcatggccggagaattatccctttgttctgctctagcagctggtcatctagtgcagagccacatgatccataatagagccaaagcttcacctacctccaccgaggttaagcaagacgatattccacgtttgcaagaaggctctgttacgtgtataaagtaa

SEQ ID NO: 20 - DNA, 1719 bp, unidentified
  source 1..1719: mol_type = other DNA
atggtcttgtcaaaatggattgttatcgctctggcacttagtgtcgcactgaatggatacttatttaatgtggctcgttggggtattaaagatccaaatgtaccagaacataatattgataggaatgaattagcgcgtgcacaacaatttaacgacacaggctccgcaactttgcccttaggtgagtatgtccctcctacgccaatgaggacacagccatcgactccagctataaccgacgacgaagcagagggtttacatatgactaaggcgagacctgctaatttacccaatagatcaaatgaagaattagaaaagttgctttcagaaaagagagtgagagaaatgacagacgaagaagtaatctcattatccatgagagggaagataccaggttacgcattagaaaagacactaggtgatttcacaagagctgtgaaaatcagacgttcaataatagctaggaataaagcaacgacggatattacacactcattggacaggagtaagttgccttatgagaattacaattgggaaagagttttcggtgcatgttgcgaaaacgtcatcggttatatgcccttgccagtcggtgtcgctggccctttagtaattgatggacaatcgtatttcattcctatggcgaccactgaaggtgttttggtagcttcggccagtagaggatgcaaggcaattaattccggcggtggggccatcactgtattgacggcggatgggatgactaggggaccttgtgtagcctttgaaacgttggaaagagctggtgcagcaaagctttggttggactcagaagccgggcaagatatgatgaagaaggcgttcaattctacatccagatttgctagattgcaatctatgaaaactgcccttgcaggcacaaatttatatataagatttaagacaaccacaggtgacgcaatgggaatgaatatgattagcaaaggagttgaacacgctttgtctgtcatggctaatgacggtggtttcgacgacatgcaaataatctccgtctcaggcaattactgtacggataagaaagctgctgcgctaaattggatagacggaagaggcaaaggtgtcgttgctgaggctataatccctggcgaggtggtgagatctgtgcttaaatccgatgtggactcgctagtagaattgaatgtcgcaaaaaacttgatcggttcagcaatggccggctctgtgggcggattcaatgcgcatgcagctaacatagtggcagccatattcctggccacggggcaggaccctgctcaagtggtagagtctgctaattgcataacgattatgaaaaacttaaacggtgcgctacaaatctcagtctctatgcccagcttggaagttggcactctgggtggaggtactatattggaaccgcaaggtgctatgttggatatacttggagtaagaggttctcatcccactaacccgggtgataatgctcgtagacttgcaagaataattggcgcggctgtactagctggtgagttgtccctgtgctccgctttggctgctggacatcttgtaagagcgcacatgcagcataataggtctgcagctcccagtagatctactactcccgctcctccaatgacacctgtttctttagcaatgaccagcgcacaggaaagatcagctagtacaacgtctatgtcagcagctgcaattcagaggtctaagtaa

SEQ ID NO: 21 - DNA, 1566 bp, unidentified
  source 1..1566: mol_type = other DNA
atgagtacaataaagtaccaccaaaacgaggcaagtgacgttatatctagttcgcctcagaagaaatctcaattcatggaaactaatcaaccttatgacaatactttgcaaacccctattaacatcgatgatgaagaagaaggtctagagatctgtttgagcaagtcaaagtcaccatcaaaaaggactcaagcgcaacttgaaatgatgttaaaagagaatcaagccagcgaattggatgatcaggaattgattgaattatcattacagggcaagattcctgggtacgcattagagaagaagttaaaagatacaactagagcggttaaaataagacgtgcggttatttctcgtacgttaacaacttctcagacgactgggttattagagtactcgaaattaccttataagaactatgattgggacagagttttaggggcgtgttgtgaaaatgttataggttacatgccattgccattaggtgtagcgggtccaataattattgattcacaatcttattttatccctatggcaacgaccgaaggtgtacttgtggcttccacttctagaggcgctaaggccattaacgcagggggaggagctgttacggtcataaccggtgatggcatgaccagaggtccttgcgtttcttttgaaacattggaacgtgctggagcggctaaagtatggcttgatagtgaaattggccaaaaaatcataacgaaagcttttaactccacgtccagatttgctaggttacaatcaatcaagacggcattagccggtacttatttatatccgaggtttaaaaccaccactggtgatgcaatgggtatgaacatgatctctaaaggtgtcgaacatgcgttgaatgtaatggccactgaagctggttttgaagacatgcagatcatctcagtaagtggtaatttttgtaccgataaaaagccagctgctatcaattggatcgacggtaggggtaaatcggttgttgcagaggcgatcattcctaaagatattgtcaaatcggtgttgaagtcaaccgtagatgccatggttgaattaaatatttctaaaaatttggtgggctctgcaatggccggctctataggtggttttaatgcccatgccgccaatatagttaccgctatttttttagccactgggcaggaccctgctcaaaacgttgaatccagcaactgtattaccttgatgaggaatttgggtggtaacctgcaaatttccgtgtctatgccatccattgaggttggtactctgggtggtggaacaattttggaaccacaaggcgcaatgttggacatgttgggtgtaaggggttcacatcccacacaccctggagaaaatgctcgtagattggctaggattattgccgcttctgttttaagtggagaattaagcctatgctccgccctagctgctggtcacttggtaaaatcacacatggcacacaacaggtctgcacctataaccagaagtaatacccctgcacaaatttcgacacacccaagtatgatcagtaccaactccatgagggaaaaacattaa

SEQ ID NO: 22 - DNA, 1584 bp, unidentified
  source 1..1584: mol_type = other DNA
atggccgctgaccaactggtgaaaaccgaagttaccaaaaagtcttttactgcacctgttcaaaaagctagcacgcctgtgttgaccaacaagactgtaatatccgggtccaaggtaaaatcactaagttctgcacagtcctctagctccggtccgagttcttcttcagaagaagatgattcgagggatattgaatcacttgataagaaaataaggcctttggaagaactggaggcactattgtctagcggtaataccaagcaattaaaaaataaagaagttgccgctctagttattcacggtaaattaccgttatacgctttggaaaaaaagttaggtgacaccactcgtgccgtcgctgtcagaaggaaagcactatcaatcctggcagaagctccagtgctagcttccgacaggctgccttataaaaattacgattatgacagggttttcggtgcatgttgcgaaaacgtaatcggatatatgccgctgcctgttggtgtcataggaccccttgttatcgatggcacctcataccatatcccaatggctactacggagggttgtttagttgcaagtgctatgagagggtgtaaggccatcaatgccggaggtggtgcgactactgtgctgactaaggatggtatgactagaggccctgtagttagatttcccactctaaagagaagtggtgcttgtaagatctggttggactcagaggaaggtcaaaacgctatcaaaaaagcatttaacagcacatctagattcgctagattacagcacattcaaacatgcttggcaggcgatcttctgtttatgaggttccgtacaactaccggcgatgcaatgggaatgaacatgatttctaagggtgtcgaatattcactgaaacagatggttgaggaatacggctgggaagacatggaagtggtctcagtctcaggaaactactgcacagataaaaagccagctgcaatcaattggattgaaggaagaggtaaatccgtcgttgcagaagctaccattcctggtgacgtggttagaaaagttcttaagagcgatgtatctgcccttgtagaattaaacattgccaagaatctagtcggaagtgcaatggcaggctctgttggtggctttaatgcacatgcagcaaatttagtcaccgcggtgttcctggcccttggacaagacccagctcaaaacgttgaaagttcaaattgcataactttgatgaaggaagtggatggtgatctaagaatttccgtgtcaatgccatccattgaagtcggtaccatcgggggaggcacggttcttgagcctcaaggtgctatgttagatcttttaggtgtaagaggcccacatgcgaccgcgcctggtacaaacgctagacaattggcaagaattgttgcctgtgcggtgttggcaggggaattaagtttgtgtgctgctttagctgcaggtcaccttgttcaatcccatatgactcataatagaaaaccagccgaaccgaccaaacctaacaatttagacgcaactgacataaatagacttaaggatggatcagttacttgtattaaatcatga

SEQ ID NO: 23 - DNA, 2412 bp, unidentified
  source 1..2412: mol_type = other DNA
atgaagaccgtcgtgataattgacgccttgagaactccaataggtaagtataagggttctctgtctcaagtttctgctgttgatttaggaacacatgtaaccacccagttgctgaagaggcattctaccattagtgaagagatagatcaagtcatatttggtaacgtgcttcaagctggtaatggtcaaaatccggcaagacaaattgccattaactcaggtctgtcacatgaaataccagctatgactgtaaatgaggtgtgtggctcaggcatgaaggcggtcattcttgctaagcaactgattcagctgggtgaagcagaagttttgatagcaggtggaattgaaaacatgtcacaagcaccaaaattgcaaaggtttaactacgagactgagagctatgatgcacctttctcgtccatgatgtacgatggtctaaccgatgcgttttccggacaagcaatgggtttaacagctgagaatgttgcagaaaagtatcatgtcacaagggaggaacaggatcaattctctgtccactctcaacttaaagcagctcaagctcaggctgaaggcatatttgcggacgaaattgcaccgcttgaagtctcaggcactttagtcgaaaaggatgagggcataagaccaaactcttctgttgagaaactgggaaccctaaaaacagtgtttaaagaagacggtacagtcactgcaggaaatgccagcactattaatgacggggccagcgccctaattatagcttcacaagaatatgcagaagcccatggacttccctatctggcgattattagagatagcgttgaggtcggcattgatcctgcttatatgggcatctcgccaatcaaagctatccagaagctattagcacgtaaccaattgaccacagaagaaattgacttatatgagatcaacgaagcatttgccgcaactagcatagtggtgcagagagagctggcgttaccagaggagaaggttaacatatacggcggtggcatctcgcttggtcatgcaattggtgctaccggtgcaaggttattaacctctcttagttaccaattgaaccaaaaagaaaagaagtacggtgtcgcatcactttgtataggtggaggtttgggtttagcaatgttgttagaacgtccccagcagaaaaagaactccagattttatcaaatgtctccagaagaaagattggcgtctttgttgaacgaaggtcaaattagtgcagatacaaaaaaagagttcgaaaacacagctctatctagccaaatagctaatcacatgattgaaaatcagatttctgaaacagaagtacctatgggcgttggtttacatttaactgtagacgaaaccgactatttagtccctatggctactgaggaaccatcagtcatagcggccctgagtaatggtgcgaaaatcgctcagggttttaagactgtgaaccaacaaagattgatgagaggtcaaattgttttttatgacgtggctgatgccgaatctttaattgatgagttgcaggtcagagaaacagaaatttttcaacaagccgaattatcttatccttcaattgttaaaagaggtggcggcttgagagatttacaataccgtgccttcgatgaatcattcgtatcagttgatttcctagtcgatgttaaggacgccatgggtgctaatatagttaatgctatgctagagggcgtagctgagttattcagagaatggtttgctgagcaaaaaatactattttccatactaagtaattatgcaacggagtcagtagtaaccatgaagacagctattcctgtatcgagattaagcaaggggagtaacggtcgtgagattgcagaaaagattgtcctagctagcaggtacgcttccctagatccatacagagctgttacacataataaaggcatcatgaatggtattgaggccgttgtcctagccacaggaaatgatacgagagctgtttctgcctcttgtcatgctttcgcagttaaagagggtaggtaccaaggtttgactagttggactctggatggtgaacaactaattggtgaaattagtgttccattggccttagctactgtaggaggcgctaccaaggtactgcctaaaagccaagctgcagcagacttgcttgctgtcactgatgccaaggaattgtctagagtggtggctgctgtgggtctagcgcaaaatttggctgctctacgtgccttggtgagtgaaggtattcaaaaaggacatatggctttgcaagctaggtctctagctatgacagtgggtgcaactggcaaagaagtagaagctgtcgcacaacagcttaagagacaaaaaaccatgaatcaagatagggctttagccatacttaacgatttgagaaaacaatga

SEQ ID NO: 24 - DNA, 1290 bp, unidentified
  source 1..1290: mol_type = other DNA
atggttgcggattctagattaccaaattttagggcactaactcctgcgcaaaggagagacttccttgcagatgcatgtggactttccgatgcagaaagggcattgctggccgccccaggtgcattgcccctagctctagcagatggcatgattgaaaacgtatttggatcctttgaactgccattaggagtagcggggaactttagagttaatggtcgtgatgtgcttgtaccaatggcagttgaagaacccagtgttgtggctgccgcctcttacatggctaaattggctagagaggatggtggtttccaaacaagttcaacactaccgttaatgagagcacaagttcaagtccttggtgttacagatccccatggtgctagattagccgtcttacaggcacgtgcacaaataattgaaagagccaattctagagataaagttttgatcggattaggcggtggttgtaaggacattgaagtgcatgtgtttccggacactccaaggggaccaatgttggtggttcacctaattgtcgatgttagggatgcgatgggtgcaaatacggtaaacacgatggctgagtcggtagcacctttggttgaaaaaataaccggtggatccgttagactgagaatattaagtaacttggctgacctacgtttggccagggctagggttagattaaccccacaaactctagcgactcaggatagatccggagaagaaattatcgagggtgttcttgacgcttatacttttgccgccattgacccctaccgtgctgccacccataacaaaggtattatgaatgggatagatcctgttatcgttgccactggtaatgactggagagcggtagaagctggtgctcacgcttatgcttcaagatctggaagctacactagcttaactagatgggaaaaagatgcaggtggtgcattggtaggttcaattgaattacccatgcctgtcggtctggtgggtggtgccactaagacgcatccactagcaagactggcacttaaaataatggacttgcaaagtgctcagcaattgggtgaaatcgcagcagctgttggtctagctcaaaacttgggtgccttaagagcacttgcgacggaggggatccaacgtggtcatatggccttacatgccagaaatatagcactggtggccggtgctacaggtgatgaagttgatgccgtagcccgtcaattagccgcagaacatgatgtgagaacagatagggcccttgaggttttagccgctctaagggccagagcataa

SEQ ID NO: 25 - DNA, 1212 bp, unidentified
  source 1..1212: mol_type = other DNA
atgactgatgccgcctctctggctgacagggtcagagaaggtgacttgcgtttacatgaattggaagcacacgctgacgcagatactgctgctgaagcaaggagattattagtagaatctcaaagtggcgcatcgttggatgcagtaggaaattacggtttccccgctgaagctgccgagagtgcaatcgaaaatatggttggttcgattcaagttcctatgggcgttgctggtccagtttccgttgacggcggttcggttgcgggagaaaaatacttgcctcttgcaactaccgaaggtgctttacttgctagtgttaacagggggtgctcagtgataaatagcgctggcggtgctacagcaagggttttaaaaagtggtatgaccagagcaccggtctttagagtagctgatgtcgcggaggcggaagcacttgttagttggacaagggacaattttgcagcccttaaagaggctgcggaagaaacaacaaatcatggagaattgctggacgtaacaccttacgtggtcggtaattcagtctatttgagattcagatatgataccaaggatgctatgggtatgaacatggctacaatcgcaactgaagctgtgtgtggtgttgttgaagcagaaacggctgccagccttgttgcattatcaggcaacttgtgttcagacaagaaacctgctgctattaatgctgtagaaggtcgtggtagaagcgttaccgctgatgtaaggattccaagggaagtggtcgaggaaagattgcacacgacccctgaagctgttgccgaattgaacaccagaaaaaatctagtcggttctgctaaggctgcatcattaggctttaatgctcatgtcgctaatgttgtagccgctatgtttcttgcgacaggtcaggatgaagcgcaggttgttgagggtgcaaacgcaatcactacggctgaggtacaagacggagatttgtacgtatcagtttccatcgcgtcgttagaagtaggtactgtaggtggtggtaccaagttgcctactcaatctgaaggtttagacatattaggagtctccggaggtggggatcctgccggttcgaatgctgacgccttagctgaatgtatcgctgttggttcattagcaggtgaattatcactattgtcggctttagcctctcgtcatctatcttctgcgcatgcagagttgggcagatga

SEQ ID NO: 26 - DNA, 2439 bp, unidentified
  source 1..2439: mol_type = other DNA
atgaaggaagtagttataatagacgccgccagaaccccaattggtaagtataagggttctttaagttcgttctctgcagtggagttaggtaccatggttacaaaaaaattattagaaaaagcaagtattaagaaagatgaaattaaccaagtgatattcggcaacgtcttacaagcaggaaatgggcagaacgtcgccaggcagatctctattatatctgacattcccgttgatgttcctgccatgactattaatgaggtgtgtgggtcaggtatgaaggctgtcattttggcaagacagctgattcaactgggtgaggccgatttagtaattgccgggggtacagaatctatgacgcgtgctcccttattacaacagttcgattctgagactactagctataacggaccaatatcctctatggtgaatgatgggttgacagacactttcagtaacacgcacatgggtttgaccgcggagaatgtcgcggaacaattcggggttaccagaaaggaacaagaccaatacgccttagattcccaattaaaggccgctaaagcaacagagaataatgtctttaaagaggaaattattccagtcactctacctgacggaacgttattagagaatgacgaagccattaggggcaactcctcattagaaaaactgggaactttaaaaacagttttctctgaaaatggtactgtgactgcagggaacgcatctccgctgaatgatggcgccagtgttatgattctggcttctaaggaatatgcactaaaaaatgatttaccctacctggccaccataaagggagtagccgaaatagggatagacccatcaattatgggaatagcccctattaacgcaatcaatagtctactggaaaaaacagatgtttctttggatgccatagatcgttttgaaattaacgaagcattcgcggcatcatccatcgtagttaatagggaactacaacttgacccagaaaaggtgaatagtgatggtggtgccatagcacttggacatcctatcggtgcaagtggtgcaagaattctgacaaccttgtcgtatgggttgcaaagaaacgaacaaaaatacggcattgcctctctatgtatcggcggaggcttaggccttgcagtattgcttgaggcgaatcaggaaaaagcaggctcatttaatgagaagaaaaaattctatcagctaactccagaggaaagaagatcccaattagtcagagggggtgtaatctctaaggaatcagccgatcagctaaaaaatgaaaggttgtctgaagacatcgctaaccatcttattgagaaccaaatctcgcaggtcgagattccgatgggggtcgcacagaattttcaaatcaatggagaaaagaaatgggtgccaatggctaccgaagaaccctctgttattgccgctgcaagtaatggggctaaaatttgcggcaacattacagcgaagaccccgcaaaggctaatgagaggacaaatcgtgctaactgggaagtctgaatatcaagctattattgaagctattgatacaaggaaggatgaactatttctttgtgctaataatagctacccttccatagtaaaaaggggcggtggtgtaagagacatctccacaagagaatttatgggatctgatcatgcttacgtatcgattgacttcctaatcgacgtaaaggatgctatgggagccaatattgtcaacgcaattttggagggtgttgcatctcaattaagatcttggtttccagatgaagagattttgttctcaattttgtcgaatctagctaccgaatccttggctactgcgtgttgtaccattccttttgaatacttgggtaagagcaaggaagctggtagacaagttgcagaaaagattcagcaagccgctgaatatgctaaattggatgtttatagagccgctactcataataagggcattatgaatggtatagaagctgtaatcttagctactggtaatgatacaagagctgcttctgccgctattcatgcatatgcgtctagaaatgggttctatcagggcctgactgattggaagatcgttgacggccagcttgttgggaagttaacagtaccgctagcagtagcaacagtcggtggtgcttctaaaattttaccgaaggcgaaattggctcttgaaatactagacgtgtcatctgcaaaggagctagctcaagtgatcgcagctgttggtttagcacaaaatttggctgcgttgaaggcccttgtcacagaaggtatccaaaagggtcatatgtccttacaggctagggctttggctattaccgtaggtgctacaggcgatgaaattgaacaggttgcgtcctaccttagaaaggccgataccatgaaccagcagttagcttccgactatctgctggaaactaggagctaa

SEQ ID NO: 27 - DNA, 1245 bp, unidentified
  source 1..1245: mol_type = other DNA
atggcttctaaaactgaaacgactatgaaggaagacgaattgctggagaaggtcgtgagtggtgaaatgccacttagaaaaattgatgcctacacagatactgatacagcagtgagagtaaggaaatgcgcaatagaaaaaatgaatggagtgaagttcgaacacattcaaaattacacaattgacgctgaggcagctacgaaaagaaacatagaaaatatgataggtacgattcagattccactaggtgtcgccggtgcaattatggtgaatggcgaatatgcatctggagaatttatgttacccctggctaccacagaaggtgcattagtcgcatctgttaataggggatgcacggttatcactgcttctgggggatctaatgtgcgtatcttccaggatctgatgaccagagccccagttttcaagttggagaatgtgaataaagttaaagaattcgttgattgggtaaagagagaggagactttcaccaatatgaaagagaaagccggagaaacaactagatttggggagttgttgtccgtcgatcccttcattactggtaacacggtttttcttagatttgcttacgatactaaggacgctatggggatgaacatggtaactatagctacagatgcggttttaaattttatttccgaggatttcggcgtgtatccgatctctttaagtggtaacatgtgtacggataaaaaaccagcggcgattaacaatattttaggcaggggaaaaactgttgctgctgatgtaactattcctaaagaaattgtcgagaaaaaattaaaaaccacaccaaagatgatggaagaagtcaactataggaagaatctgttgggctctgcaagggctggtgccctgggcttcaacgctcacgcggctaatataatagccgcattatatttggcttgcggccaagatgcggcacatgttgtcgaagggtctagtgccattactacaatggaagtaaatgaaaatggtgatttgtactgttcggttacactacctagcatacaagtaggtacagtcggtggaggtactggtatcgccactcaaagagattgcctaaatttgctgggtgtagctggagctggtgaggtacctggtcataattcaaaaaagctagctgaaattattgctgccgcagtcctggctggagaaatctccttgattggtgctcaagcagctggccaccttgctaaagcacacgccgaattgggtagataa

SEQ ID NO: 28 - DNA, 1257 bp, unidentified
  source 1..1257: mol_type = other DNA
atgtttttaaaagataatgatcttacagaagatgaaaaattgttgttgcaaaaggttttggatggtgacatcgcttttaggaagatcgaggaatttgcagacccgcttacggcagtcaagattcgtcgtttagctatacaggagtacgccaaattggaatttgaacacatccaaaatttctcattggacgttgaaactgtaactaagaaaaatattgaaaacatgatcggagccgttcaaataccattaggggttgccgggttactaaaggttaatggtgagtacgctgacgcagagtactacattccattagctacaacagaaggcgccctggtagctagtgtaaatagaggctgttcagtaattactaaatcaggaggtgctaatgttagagtattcgaggatgaaatgactagggctccggtctttaaacttgaaagtttagatagaaccaaaaagttctatgagtgggttaaaagtcccgagatctttgaacaaatgaagactgttgcggagaagacgacaagatttggtaaattattgtctgttaagccattcgtgaccggcacttatgtttatcttagattctcgtacgataccaaggacgccatgggcatgaatatggtgactatagcaacggatgctgttatgcatcttattgaagacgaatttggtgcccaccctatcacactaagtggtaatatgtgcaccgataaaaaaccagcttcaatctctaccatcttaggaagaggtaaaactgtagtcgctgaggttactatccccgaagaaattgtaaaggaaacgttgaagtgtacaccagacgctatgttcgaagtcaattacagtaaaaatctactagggtctgcaagagccggtgcgttaggttttaacgctcacgccgccaacgttatagctgcagtgtacttagcatgtggtcaagatgcagcgcatgtagtggaaggttccactgctataaccagtatggaattaactaaatatggtgagattcactgttcagtgaccttacccgccttgcctgttggtactgtgggaggcggcacgggattgggtacccaaagggattgtttgaatatattaggcgtcgccggtactggggatataccaggcattaattcaagaaaattcgccgaaattgttgctagtgcagttttagccggcgagatttcgttgatcggtgcgcaggcagctggacacctggctcgtgcccacgcccaattgggtagaggaaaattctaa

SEQ ID NO: 29 - DNA, 1287 bp, unidentified
  source 1..1287: mol_type = other DNA
atgtctttagacagtaggttacctgcttttagaaatttatcccctgctgccagattggatcatataggtcaattgcttgggctatcacacgacgacgtttctcttctagctaatgcaggagccttgccgatggacatagctaatggtatgatcgaaaatgtgattggtacctttgaattgccctacgcagtagcttctaactttcagataaacggtagggacgtacttgtaccattggtggttgaagagccctcaatagtcgctgctgcttcatacatggccaagttagcccgtgccaacggtggtttcactaccagctcatcggctccattgatgcacgcacaagtccagatagtcggtatccaagatccactgaatgccagactgtctttgttgagaagaaaggacgaaatcatagagttagcaaatagaaaggatcaacttttaaactcattaggaggtggctgtcgtgatatcgaggtacatacttttgccgacactccaagaggccctatgttggtcgcacatttaatagttgatgtacgtgatgctatgggcgcgaatacagtgaacacaatggccgaagcggtagctccattgatggaagccataacaggaggtcaagttcgtctacgtattttatcaaacctagcggatctgagattggcaagagctcaagtgaggatcaccccgcaacaacttgaaactgctgaatttagcggtgaggctgtgattgaaggtatccttgacgcttatgcgtttgcggcagtagatccctatcgtgctgcaacccacaacaagggcattatgaatgggattgacccgttgatagtggcaacgggtaacgattggagagcagttgaagcgggtgctcatgcttatgcatgtagatcaggacattatggtagtctaactacctgggagaaagataacaatggacatttggttggtacactagaaatgcctatgccagttggtttagtcggaggcgccactaaaacacatcctttggcacaactatctttaagaatattgggtgtaaagactgcccaagccttggcagaaattgccgtcgcagtcggtttagcgcaaaatcttggagctatgagagcactggccaccgaaggcatccaacgtggccatatggcattgcatgccagaaatattgcagtcgttgctggggccaggggagacgaagtggattgggttgctagacaattagtagaataccatgacgttagggctgatagagcggttgcccttttaaagcaaaagagaggacaatga

SEQ ID NO: 30 - DNA, 1272 bp, unidentified
  source 1..1272: mol_type = other DNA
atgacaaagctatcttggacggggtttagtaagaagacattgcaagaaagaaaggagcacctgaaaaataatgcattgttatcccaagaaaatcaagatttgcttgacaatgaccaacaacttactctagagaccgcaaatcaaatggcagaaaatgtcataggaagatttacattgccttttgcaatatgcccagacgtactagtggacggtgtaacataccaagttccgatggttactgaggaacctagtgttgttgcagctgcctcatatgctagcaaactaatcaaaagatcaggtggttttacaactaagatacatgatagacagatgattggccaagtagccctatttgatgttcccgataaggccacggctgcctcaaaaatacaggcagcgtcacaaaagttaattgatatcgctaaggaagcgtacccttccatagtcaaaagaggaggcggtcctagaaaattgtggaccgagacaaaaggagattttttaatagtgtatttagcagttgacacacaagaagcgatgggtgcaaatatggttaatacaatgatggaagcgttggttccagaattggaaaatttgtctgagggacaatcattaatggccattttgtcaaatttggctaccgaatccctggtaacagcaacatgtaggctgaacactagattcctatctagaaataaggccgaagctcacaacttcgcaaaaaaaatggaattagcttcccagttggcacaagtggatccgtacagagcagcaacccataataagggtattttcaatggaatagacgcattggttatcgctacaggaaacgattggagagctgtcgaagctgggtgccatgcttatgctagtaaggatggttcttatagaggcctttctacatggacgtataatcaggaaaccaaagaattggttggtgaattgacactgcctatgcctatcgctacaagaggtggttctattggtctgaatcctagtgtttcaatagcacatgatcttctgaatcaccccgatgctagaacgctagccggtatcatagtgtcactaggtctggtccagaatcttgctgcgctgaaggctcttactagcactggcatacaggcaggccatatgaaattacaagctaagtccttggcgttgctggctggggcgaatccggaagaaatgccccacgtgttgtctgaattacttaaagcaaaacatatgaaccaagaaacagctcaagcaatcctggaaaaacttcgtaatccgtag

SEQ ID NO: 31 - DNA, 2427 bp, unidentified
  source 1..2427: mol_type = other DNA
atgaaggacgtcgtaataattgatgccctaagaacgccggtgggaaagtatcaagggtctttatcacagttaagtgccgttgaattaggctctgccgtgtctaagaaattaataaataacaataagaaggcggcagccgcaattaatcaggtgatatttggaaacgttttgcaagcaggatctggacagaatccagcacgtcaaatcacgttaaattcaggactatctgaatcggtatatgccagtaccattaatgaagtgtgtggctctggtatgaaagcgatttctcttgcatcccaagctatttttttagatgaagctgaagttgtgttagctggcggaacagaatctatgtcgcaagcgccttatttaagctattacaatcaacaggaagatacgtatagtcaaccaaagcctgctatgttgtctgatggtttgactgatgtgtttagtggccaacacatggggctgactgccgagaatgtagccgagaagtttaacatcaccagaaaaatgcaggatgcgttcgcccttagatctcaagagagagccgcaaatgcccaggagaaaggctattttagcaatgaaatacttccgctagatatagcaggtaagaaggtggacaaagacgaaggtgttcgtaaagatacaagcttagaaaagctagcaaagttaaaaactgtcttcaaaaaagaaggcacggtgacggctggaaacgcttctacaattaacgatggggcgtcggctgtgcttttggcctcaaagaactttgcattagccaacgatttatcctacttggcagtcttgaaggatgttgtggaagtaggtgtcgaccctaaagttatgggtatttcacccataaaggccattaggcaattgctagaaagaaatgccttggctattgagaatattgatttgtttgaaattaatgaagcgttcgcttcgtctagtatagcggtagagcaagaactagaaattcctgaagataaggttaacgtgtgtggttcgggcatctctatcggccacgctataggcgcatctggcgcaaggattattacaacagcttgtcatcaattggaaagagttgacggcaggtatgcagttgtttctttgtgtgttggtggtggtttgggattagccgcactaatagaaagaccaaaagctaataagagccacaaattttatcaattgactcgtaaggagcgtttggatttcctggtcagtcataataagattacttcaaaaactgttgacgaattagaacgtactgtcttacctgaatcaatcgcgggtaacttaacagagaatcagatgtctgaaatttccttacccatgggcttggtatcaaatatgagcgtaaatcaaaaggattactttgtacctatggcaactgaggaaccttcagtagttgctgcatgcaacaatggtgtccaaatggcaaaatcgagtggtggttttactgcagtcatgaaaaagaaagaaatcaggggacaaattgtcttaatgaatgtcaccgacaaatccacagttatagaacagatagaaaaaaatgaagccgagattatatccactgctgagcaaagttacccttctatcgtgaagaggggtggcggagtcaagagagtcgtggtcagagagtttgctgaagaccctaatttcttaagtgttgatttaatagttgacactcaggatgccatgggtgctaatatgttaaatactatgttagaagctgttgcaactctgtttaggcaatggttttcggaagagatattgttctcaattttatcaaactacgcaactgatgctttggtatcggctgaatgctatatctcctttgccagtttgggaaaaggtgatgctgaaaagggggagaaaatagcagaaaagatagctgcagctagtaacttcgctcaaatagaccctttcagagccgctacacacaataaaggtattatgaatggtatagacgctgtcgttcttgctacggggaacgatactcgttctgtgaatagtgccgtacatgcttatgcagctaagaacggtaagtatcaaggtttatcccaatgggagattgtagataaccagctgaaaggatctatagaattgcctttagcggtcgccactgctggtggggcgactaaagtactgccaaaagcgcaggcagcgctgcagatcttggatgtaaatgatgccaaagaattagcagaagtcatcgcatccgtcggtctagcacaaaacctagccgcattaaaagcccttgttactgagggtatacaaaagggacatatggccttgcaggcaaggacattggctctttccgtaggagcaaaagactccgaagttcagaaagttgctaatagattgaaacgtcaacaaatgaatgaggaaaatgctagaaaaattttgcaggaactacgtaaccgttag

SEQ ID NO: 32 - DNA, 1740 bp, unidentified
  source 1..1740: mol_type = other DNA
atggaagttagaggtggtgtgggacagggctcagctgctagacatcctccagctcccgagcctagtcgtgctgctgccagggtgcaggctggtgacgctctaccgttacccataagacatacaaacttgatttttagtgctctgtttgcagcgtcattggcctatttgatgcgtcgttggagagaaaaaataagatcatcaacacctcttcatgccgtgggattggccgaaatgttggcaattttcggactggtagcttcactaatttatcttctgagctttttcggcattgccttcgtacaatctattgtttccagcggagatgatgacgaagacttcttggtcggttctggatcttcaggatcggctgcagcgccctcaagacaacatgctcaagcaccagcgccatgtgaacttctgggtagtccagcagctgcaccagagaagatgcctgaggatgatgaagaaattgtggccagtgtagtggctggaaaggtcccgagttacgctcttgaggcaagattgggtgattgcagaagagctgccgggattaggcgtgaggcattaagaagaattactggtagggatatcgaagggttacccttggatggcttcgactatgcatccatcctaggtcagtgttgtgagttgccagttggttacgtacaattaccagtcggggtagctggacctttattattagatggacgtagattttacctgccgatggcgaccacggaaggttgtttggttgcaagcacaaatagaggatgtaaggctattgctgaatctggtggtgctacatctgtagtgttgagggatgcaatgactcgtgctccagtcgctcgttttccgaccgctcgtagagccgctgaactaaaggcttttttggaagatccagctaatttcgatacattaagtgtggtatttaatagaagctcccgttttgcaagattacaaggcgtccaatgcgcaatggctggtagaaatttgtacatgaggttttcctgtagcacaggtgacgcgatgggtatgaacatggtttccaagggagttcaaaatgttttagattttcttcaggatgactttcacgatatggacgtaattagcatttccggtaatttttgctcagacaaaaaaccttctgctgtcaattggattgagggtagaggaaagtctgttgtatgcgaagcggtcattggtgaagaagttgtaaaaaaagtgttaaaaacagatgttcaatctttagtagaattaaacactataaaaaatctagctggttctgccgttgccggtgctttagggggcttcaacgctcacgctagcaatatcgttactgccatttttattgcgaccggtcaagatccagcccaaaatgtcgagagtagtcattgtatcacaatgctggaaccagtcaacgcaggtagagaccttcatatatcagtaaccatgccttcaattgaggtcggcactgttggtgggggtactcaactagcctcgcagtctgcttgcttagatctattgggtgttagaggcgcgtcgagggacaggcccggttcgaatgcaagacttctagctacagttgtcgcggggggtgtgttagccggtgagttgtctttgctttcagccctagctgccggccaattggttaaatcccatatgaaatataataggagttccaaagatgtttctagtaccaccgcaactgaaaagacaagacaacgtgaagtggatgtttga